(19) United States
(12) Patent Application Publication
(10) Pub. No.: US 2024/0002332 A1
(43) Pub. Date: Jan. 4, 2024

(54) Small Molecule Inhibitors of SARS-CoV-2 Infections

(71) Applicants: Wenwei Huang, Bethesda, MD (US); Khalida Shamim, Bethesda, MD (US); Hengli Tang, Tallahassee, FL (US); The USA, as represented by The Secretary, Dept. of Health and Human Services, Bethesda, MD (US); The Florida State University Research Foundation, Tallahassee, FL (US)

(72) Inventors: Wenwei Huang, Bethesda, MD (US); Khalida Shamim, Bethesda, MD (US); Hengli Tang, Tallahassee, FL (US)

(21) Appl. No.: 18/253,031

(22) PCT Filed: Nov. 18, 2021
(86) PCT No.: PCT/US2021/059910

§ 371 Date: May 15, 2023

Related U.S. Application Data
(60) Provisional application No. 63/115,288, filed on Nov. 18, 2020.

(51) Int. Cl. C07C 235/84; C07D 211/46; C07D 209/26; C07D 277/44; A61P 31/14; A61K 9/00; C07D 215/50

(52) U.S. Cl. CPC C07C 235/84; C07D 211/46; C07D 209/26; C07D 277/44; A61P 31/14; A61K 9/0019; C07D 215/50

(57) ABSTRACT

The present disclosure relates generally to compounds, pharmaceutical compositions comprising them, and methods of using the compounds and compositions for treating disease caused by severe acute respiratory syndrome coronavirus 2 (SARS-CoV-2), commonly referred to as coronavirus disease 2019 (COVID-19). The present disclosure relates more particularly to small molecule inhibitors of SARS-CoV-2 of Formula (II) and prodrug derivatives thereof of Formula (I), and methods of treating COVID-19 therewith.

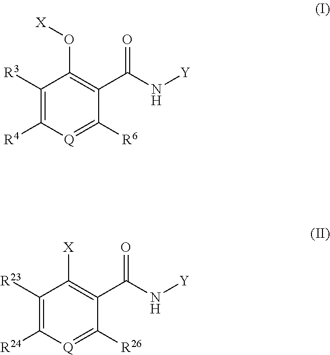

### CROSS REFERENCE TO RELATED APPLICATIONS

This application claims priority to and the benefit of U.S. Provisional Patent Application No. 63/115,288, filed Nov. 18, 2020, the contents of which are hereby incorporated by reference in their entirety.

### FIELD

The present disclosure relates generally to compounds, pharmaceutical compositions comprising them, and methods of using the compounds and compositions for treating disease caused by severe acute respiratory syndrome coronavirus 2 (SARS-CoV-2), commonly referred to as coronavirus disease 2019 (COVID-19). The present disclosure relates more particularly to small molecule inhibitors of SARS-CoV-2 and prodrug derivatives thereof, and methods of treating COVID-19 therewith.

### TECHNICAL BACKGROUND

In late 2019, a novel coronavirus named Severe Acute Respiratory Syndrome Coronavirus 2 (SARS-CoV-2) was identified after a cluster of patients presented with severe pneumonia in Wuhan city, in Hubei province of China. The epidemic disease caused by SARS-CoV-2 was named coronavirus disease 2019 (COVID-19) by the World Health Organization (WHO). The disease spread quickly from Hubei province to the rest of the world, and by spring of 2020 was reported in almost every country, resulting in global pandemic. Importantly, the COVID-19 pandemic is showing no signs of subsiding and, as of October 2020, there were over 40 million confirmed cases globally, of which over one million resulted in death.

A significant proportion of SARS-CoV-2 infections (estimated 40-45%) occur without symptoms and that infection can be spread by people showing no symptoms. Available data indicate that SARS-CoV-2 has principally spread through exposure to respiratory droplets carrying infectious virus within a short range (e.g., less than six feet). To stop the efficient and rapid spread of SARS-CoV-2, several groups are focusing on developing effective vaccines. But parallel to vaccine development, there is a dire need for antiviral therapeutic agents that can significantly reduce morbidity, mortality, and deleterious long-term effects associated with patients afflicted with COVID-19.

Although several therapeutic agents have been evaluated for the treatment of COVID-19, no antiviral agents have yet been shown to be significantly efficacious. Remdesivir (GS-5734), an inhibitor of the viral RNA-dependent RNA polymerase (available from Gilead Sciences, Foster City, California), remains as the only approved antiviral agent in the U.S. against SAR-CoV-2. Remdesivir shows a median recovery time of 10 days, compared to 15 days in those who received placebo (Beigel et al. “Remdesivir for the Treatment of Covid-19-Final Report,” N Engl J Med, Oct. 8, 2002; DOI: 10.1056/NEJMoa2007764). The Kaplan-Meier estimates of mortality by day 15 were 6.7% with remdesivir and 11.9% with placebo, and by day 29 were 11.4% with remdesivir and 15.2% with placebo. While this is a crucial milestone, there is significant room for improvement. There remains a need for effective therapeutic agents for treatment of COVID-19.

### SUMMARY

In one aspect, the disclosure provides a compound of Formula I

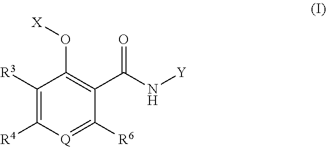

or a pharmaceutically acceptable salt thereof, wherein
X is —PO(R^{1A})(R^{2A}); and
   R^{1A} and R^{2A} are each independently C₁-C₆ alkoxy;
or X is —C(O)R^{1B}; and
   R^{1B} is C₁-C₁₂ alkyl, C₁-C₁₂ haloalkyl, hydroxy(C₁-C₈ alkyl), alkoxy(C₁-C₈ alkyl), or amino(C₁-C₈ alkyl), cycloalkyl optionally substituted with one or more R¹⁴, aryl optionally substituted with one or more R¹⁴, heteroaryl optionally substituted with one or more R¹⁴, or heterocycloalkyl optionally substituted with one or more R¹⁴;
or X is —C(O)NR^{1C}R^{2C}; and
   R^{1C} and R^{2C} are each independently hydrogen, C₁-C₆ alkyl, C₁-C₆ haloalkyl, hydroxy(C₁-C₆ alkyl), alkoxy(C₁-C₆ alkyl), or amino(C₁-C₆ alkyl), or
      R^{1C} and R^{2C}, together with the atoms to which they are attached, form a 5- to 8-membered monocyclic heterocyclyl optionally substituted with one or more R¹⁵ selected from C₁-C₆ alkyl, —NH₂, —NH(C₁-C₆ alkyl), —N(C₁-C₆ alkyl)₂, —OH, C₁-C₆ alkoxy, cycloalkyl, or heterocycloalkyl;
      or R^{1C} and R^{2C}, together with the atoms to which they are attached, form a bicyclic heterocyclyl selected from a 5- to 8-membered monocyclic heterocyclyl bridged with 1-3 additional carbon atoms, and a 5- to 8-membered monocyclic heterocyclyl fused to a monocyclic cycloalkyl, monocyclic aryl, a monocyclic heterocycloalkyl, or a monocyclic heteroaryl;
R³ and R⁴ are each independently hydrogen, halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, C₁-C₆ haloalkoxy, —SH, —CONH₂, —CONH(C₁-C₆ alkyl), —CON(C₁-C₆ alkyl)₂, —CO₂H, —CO₂(C₁-C₆ alkyl), —CHO, —CO(C₁-C₆ alkyl), or —S(O)₀₋₂(C₁-C₆ alkyl), or
   R³ and R⁴, together with the atoms to which they are attached, form a 5- or 6-membered aryl, heteroaryl, or heterocyclyl optionally substituted with one or more R¹⁴;
Q is N;
or Q is CR⁵; and
   R⁵ is hydrogen, halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, C₁-C₆ haloalkoxy, —SH, —CONH₂, —CONH(C₁-C₆ alkyl), —CON(C₁-C₆ alkyl)₂, —CO₂H, —CO₂(C₁-C₆ alkyl), —CHO, —CO(C₁-C₆ alkyl), —S(O)₀₋₂(C₁-C₆ alkyl), cycloalkyl optionally substituted with one or more R¹⁴, aryl optionally substituted with one or more R¹⁴, heteroaryl optionally substituted with one or more R¹⁴, or heterocycloalkyl optionally substituted with one or more R¹⁴;
R⁶ is hydrogen, halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, C₁-C₆ haloalkoxy, —SH, —CONH₂, —CONH(C₁-C₆ alkyl), —CON(C₁-C₆ alkyl)₂, —CO₂H, —CO₂(C₁-C₆ alkyl), —CHO, —CO(C₁-C₆ alkyl), —S(O)₀₋₂(C₁-C₆ alkyl), cycloalkyl optionally substituted with one or more R¹⁴, aryl optionally substituted with one or more R¹⁴, heteroaryl optionally substituted with one or more R¹⁴, or heterocycloalkyl optionally substituted with one or more R¹⁴, or
   R⁵ and R⁶, together with the atoms to which they are attached, form a 5- or 6-membered aryl, heteroaryl, or heterocyclyl optionally substituted with one or more R¹⁴;
Y is

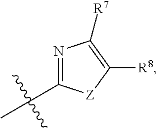

wherein
   Z is O or S;
   R⁷ and R⁸ are each independently hydrogen, halogen, —NO₂, —CN, C₁-C₆ alkyl, or C₁-C₆ haloalkyl;
or Y is

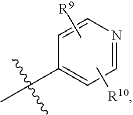

wherein
   R⁹ and R¹⁰ are each independently hydrogen, halogen, —NO₂, —CN, C₁-C₆ alkyl, or C₁-C₆ haloalkyl;
or Y is

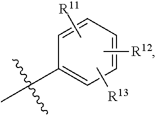

wherein
   R¹¹, R¹², and R¹³ are each independently hydrogen, halogen, —NO₂, —CN, C₁-C₆ alkyl, C₁-C₆ haloalkyl, —NH₂, —NH(C₁-C₆ alkyl), —N(C₁-C₆ alkyl)₂, —NHCO(C₁-C₆ alkyl), —N(C₁-C₆ alkyl)CO(C₁-C₆ alkyl), —OH, C₁-C₆ alkoxy, C₁-C₆ haloalkoxy, —SH, —CONH₂, —CONH(C₁-C₆ alkyl), —CON(C₁-C₆ alkyl)₂, —CO₂H, —CO₂(C₁-C₆ alkyl), —CHO, —CO(C₁-C₆ alkyl), —S(O)₀₋₂(C₁-C₆ alkyl), —S(O)₀₋₂NH₂, —S(O)₀₋₂NH(C₁-C₆ alkyl), or —S(O)₀₋₂N(C₁-C₆ alkyl)₂, or
      R¹¹ and R¹³, together with the atoms to which they are attached, form a 5- or 6-membered aryl, heteroaryl, or heterocyclyl optionally substituted with one or more R¹⁴; and
each R¹⁴ is independently halogen, —NO₂, —CN, —NH₂, —NH(C₁-C₄ alkyl), —N(C₁-C₄ alkyl)₂, —OH, C₁-C₄ alkyl, C₁-C₄ haloalkyl, C₁-C₄ alkoxy, or C₁-C₄ haloalkoxy.

In one embodiment, the compound of Formula I is not 4-chloro-2-((2-chloro-4-nitrophenyl)carbamoyl)phenyl octanoate, 4-chloro-2-((2-chloro-4-nitrophenyl)carbamoyl)phenyl acetate, 4-chloro-2-((2-chloro-4-nitrophenyl)carbamoyl)phenyl benzoate, 4-chloro-2-((2-chloro-4-nitrophenyl)carbamoyl)phenyl diethyl phosphate, or 4-chloro-2-((2-chloro-4-nitrophenyl)carbamoyl)phenyl 4-methylpiperazine-1-carboxylate.

In another aspect, the disclosure provides a compound of Formula II

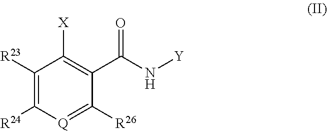

or a pharmaceutically acceptable salt thereof, wherein
X is —OR²¹, wherein
   R²¹ is hydrogen, C₁-C₆ alkyl, C₁-C₆ haloalkyl, hydroxy(C₁-C₆ alkyl), alkoxy(C₁-C₆ alkyl), amino(C₁-C₆ alkyl), cycloalkyl, heterocycloalkyl,
or X is —NHR²², wherein
   R²² is hydrogen, C₁-C₆ alkyl, C₁-C₆ haloalkyl, hydroxy(C₁-C₆ alkyl), alkoxy(C₁-C₆ alkyl), amino(C₁-C₆ alkyl), —CHO, —CO(C₁-C₆ alkyl), —S(O)₀₋₂(C₁-C₆ alkyl), or —S(O)₀₋₂(aryl);
R²³ and R²⁴ are each independently hydrogen, halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, C₁-C₆ haloalkoxy, —SH, —CONH₂, —CONH(C₁-C₆ alkyl), —CON(C₁-C₆ alkyl)₂, —CO₂H, —CO₂(C₁-C₆ alkyl), —CHO, —CO(C₁-C₆ alkyl), or —S(O)₀₋₂(C₁-C₆ alkyl), or
   R²³ and R²⁴, together with the atoms to which they are attached, form a 5- or 6-membered aryl, heteroaryl, or heterocycyl optionally substituted with one or more R³⁴; or
   X is —NHR²², and R²² and R²³, together with the atoms to which they are attached, form a 5- or 6-membered heteroaryl;
Q is N;
or Q is CR²⁵; and
   R²⁵ is hydrogen, halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, C₁-C₆ haloalkoxy, —SH, —CONH₂, —CONH(C₁-C₆ alkyl), —CON(C₁-C₆ alkyl)₂, —CO₂H, —CO₂(C₁-C₆ alkyl), —CHO, —CO(C₁-C₆ alkyl), —S(O)₀₋₂(C₁-C₆ alkyl), cycloalkyl optionally substituted with one or more R³⁴, aryl optionally substituted with one or more R³⁴, heteroaryl optionally substituted with one or more R³⁴, or heterocycloalkyl optionally substituted with one or more R³⁴;
R²⁶ is hydrogen, halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, C₁-C₆ haloalkoxy, —SH, —CONH₂, —CONH(C₁-C₆ alkyl), —CON(C₁-C₆ alkyl)₂, —CO₂H, —CO₂(C₁-C₆ alkyl), —CHO, —CO(C₁-C₆ alkyl), —S(O)₀₋₂(C₁-C₆ alkyl), cycloalkyl optionally substituted with one or more R³⁴, aryl optionally substituted with one or more R³⁴, heteroaryl optionally substituted with one or more R³⁴, or heterocycloalkyl optionally substituted with one or more R³⁴, or
   R²⁵ and R²⁶, together with the atoms to which they are attached, form a 5- or 6-membered aryl, heteroaryl, or heterocyclyl optionally substituted with one or more R³⁴;
Y is

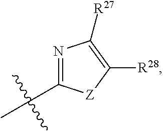

wherein
   Z is O or S;
   R²⁷ and R²⁸ are each independently hydrogen, halogen, —NO₂, —CN, C₁-C₆ alkyl, or C₁-C₆ haloalkyl;
or Y is

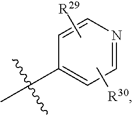

wherein
   R²⁹ and R³⁰ are each independently hydrogen, halogen, —NO₂, —CN, C₁-C₆ alkyl, or C₁-C₆ haloalkyl;
or Y is

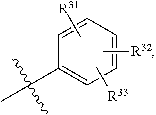

wherein
   R³¹, R³², and R³³ are each independently hydrogen, halogen, —NO₂, —CN, C₁-C₆ alkyl, C₁-C₆ haloalkyl, —NH₂, —NH(C₁-C₆ alkyl), —N(C₁-C₆ alkyl)₂, —NHCO(C₁-C₆ alkyl), —N(C₁-C₆ alkyl)CO(C₁-C₆ alkyl), —OH, C₁-C₆ alkoxy, C₁-C₆ haloalkoxy, —SH, —CONH₂, —CONH(C₁-C₆ alkyl), —CON(C₁-C₆ alkyl)₂, —CO₂H, —CO₂(C₁-C₆ alkyl), —CHO, —CO(C₁-C₆ alkyl), —S(O)₀₋₂(C₁-C₆ alkyl), —S(O)₀₋₂NH₂, —S(O)₀₋₂NH(C₁-C₆ alkyl), or —S(O)₀₋₂N(C₁-C₆ alkyl)₂, or
      R³¹ and R³³, together with the atoms to which they are attached, form a 5- or 6-membered aryl, heteroaryl, or heterocyclyl optionally substituted with one or more R³⁴; and
each R³⁴ is independently halogen, —NO₂, —CN, —NH₂, —NH(C₁-C₄ alkyl), —N(C₁-C₄ alkyl)₂, —OH, C₁-C₄ alkyl, C₁-C₄ haloalkyl, C₁-C₄ alkoxy, or C₁-C₄ haloalkoxy.

In one embodiment, the compound of Formula II is not 5-chloro-N-(2-chloro-4-nitrophenyl)-2-hydroxybenzamide.

In another aspect, the disclosure provides a pharmaceutical composition including one or more compounds of the disclosure as described herein and a pharmaceutically acceptable carrier, solvent, adjuvant, or diluent.

In another aspect, the disclosure provides methods of treating disease caused by severe acute respiratory syndrome coronavirus 2 (SARS-CoV-2), commonly referred to as coronavirus disease 19 (COVID-19). Such methods include administering to a subject in need thereof one or more compounds of the disclosure as described herein or a pharmaceutical composition of the disclosure as described herein.

Additional aspects of the disclosure will be evident from the disclosure herein.

### BRIEF DESCRIPTION OF THE DRAWINGS

The accompanying drawings are included to provide a further understanding of the compositions and methods of the disclosure, and are incorporated in and constitute a part of this specification. The drawings illustrate one or more embodiment(s) of the disclosure and, together with the description, serve to explain the principles and operation of the disclosure.

FIG. 1 is a graph showing the rescue effect of compounds 6, 39, 32, and 4 described herein against SARS-CoV-2 infection in Vero E-6 cells.

FIG. 2 is a graph showing the rescue effect of compounds 59, 27, and 24 described herein against SARS-CoV-2 infection in Vero E-6 cells.

FIG. 3A and FIG. 3B are a set of graphs showing the cytotoxicity of compounds 6, 39, 32, 4, 59, 27, and 24 described herein in Vero E-6 cells.

FIG. 4A, FIG. 4B, and FIG. 4C are a set of graphs comparing the cytopathic effect and cytotoxicity of compounds 59, 24, and 27 described herein in SARS-CoV-2-infected Vero E-6 cells.

FIG. 5A, FIG. 5B, FIG. 5C, and FIG. 5D are a set of graphs comparing the cytopathic effect and cytotoxicity of compounds P1, P2, P8, and P6 described herein in SARS-CoV-2-infected Vero E-6 cells.

FIG. 6A, FIG. 6B, FIG. 6C, and FIG. 6D are a set of graphs comparing the cytopathic effect and cytotoxicity of compounds P5, P7, P9, and P4 described herein in SARS-CoV-2-infected Vero E-6 cells.

FIG. 7 is a graph comparing the cytopathic effect and cytotoxicity of compound P3 described herein in SARS-CoV-2-infected Vero E-6 cells.

FIG. 8A, FIG. 8B, FIG. 8C, and FIG. 8D are a set of graphs showing the response of a SARS-CoV-2 viral titer in response to dose escalation of niclosamide and compound 6 described herein in human bronchial epithelial cells.

FIG. 9A, FIG. 9B, FIG. 9C, and FIG. 9D are a set of graphs showing the lactate hydrogenase LDH-glo luminescence of compound 6 described herein as a function of cell viability.

FIG. 10A and FIG. 10B are a set of graphs showing the pharmacokinetic distribution in plasma and lung for Compound P3 described herein, administered intravenously (3 mpk) or orally (30 mpk) to male hamsters.

FIG. 11A and FIG. 11B are a set of graphs showing the ratio of Compound P3 described herein in lung and plasma, administered intravenously (3 mpk) or orally (30 mpk) to male hamsters.

FIG. 12A and FIG. 12B are a set of graphs showing the pharmacokinetic distribution of active metabolite, niclosamide, in plasma and lung when Compound P3 was administered intravenously (3 mpk) or orally (30 mpk) to male hamsters.

FIG. 13A and FIG. 13B are a set of graphs showing the ratio of active metabolite niclosamide present in lung and plasma when Compound P3 was administered intravenously (3 mpk) or orally (30 mpk) to male hamsters.

FIG. 14A and FIG. 14B are a set of graphs showing the PK plasma profile of Compound P3 and niclosamide, respectively, in mice administered Compound P3 orally at 50 and at 100 mg/kg.

FIG. 15A shows the mean plasma concentration vs time profile of niclosamide after oral administration of Compound P3 at three different doses (25 mg/kg, 50 mg/kg, and 100 mg/kg) in C57BL/6J mouse for seven (7) days. FIG. 15B shows daily mouse body weights over seven (7) days for the same experiment.

FIG. 16A and FIG. 16B are a set of graphs showing the plasma, lung and liver PK profile of Compound P3 and niclosamide in mice dosed with compound P3 intravenously and orally (at 3 and 10 mg/kg respectively).

FIG. 17 shows the results of the evaluation of administration of Compound P3 in a Golden Syrian Hamster Efficacy Model of SARS-CoV-2.

### DETAILED DESCRIPTION

Before the disclosed processes and materials are described, it is to be understood that the aspects described herein are not limited to specific embodiments, and as such can, of course, vary. It is also to be understood that the terminology used herein is for the purpose of describing particular aspects only and, unless specifically defined herein, is not intended to be limiting.

In view of the present disclosure, the methods and compositions described herein can be configured by the person of ordinary skill in the art to meet the desired need. In general, the disclosure materials and methods provide improvements in treatment of disease caused by SARS-CoV-2.

Niclosamide, 5-chloro-N-(2-chloro-4-nitrophenyl)-2-hydroxybenzamide,

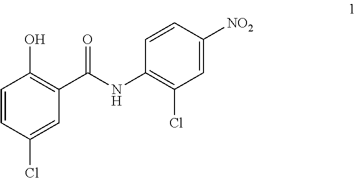 [SMILES: O=C(Nc1ccc([N+](=O)[O-])cc1Cl)c1cc(Cl)ccc1O]

was approved by FDA for use in humans to treat tapeworm infections in 1982 and is included on World Health Organization's list of essential medicines. Niclosamide is also categorized as a FDA Category B drug which implies that it did not demonstrate risk to the fetus in animal reproduction studies, although there are no adequate and well-controlled studies in pregnant women. Niclosamide has been reported to have a number of biological activities such as anti-tuberculosis activity, anti-bacterial activity, drug-resistant Staphylococcus aureus activity, anti-cancer activity, and has also displayed anti-viral activity against several viruses. For example, niclosamide was discovered in a screen for orthosteric inhibitors that directly target the interaction between the mosquito-borne flavivirus (ZIKV) proteases NS2B and NS3. Niclosamide inhibited ZIKV growth at early stages and 24 h post infection and reduced ZIKV titer in human placental epithelial cells and iPSC-derived human neural progenitor cells.

Although the pharmacokinetic properties of niclosamide are favorable for use as an antihelminthic drug, its low solubility, low bioavailability, and poor plasma exposure limit its use in methods that require oral dosing and/or systemic exposure. Although niclosamide displays good rat liver microsomal stability (>30 min) in Tier I absorption, distribution, metabolism and excretion (ADME) assays, it displays poor kinetic solubility (1.1 μg/mL) and poor permeability in parallel artificial membrane permeability assay (PAMPA) (unable to be quantified in the acceptor compartment of the PAMPA setup indicating that the compound might be trapped in the artificial lipid membrane). Historically, the restricted exposure of niclosamide has not been an issue because of its use as an antihelminthic drug, but the restricted exposure presents a significant hurdle in its use as an antiviral.

The present inventors have determined that the compounds of the disclosure can be better delivered in vivo, advantageously improving systemic exposure and/or mitigating metabolic liability relative to niclosamide. For example, in certain embodiments, intravenous or oral administration of compounds of Formula I (i.e., as otherwise described herein) can desirably provide a high concentration of available, active metabolite, and moreover can provide a relatively high concentration of active metabolite in lung tissue (e.g., relative to plasma concentration). The inventors have also determined that the compounds of the disclosure desirably inhibit SARS-CoV-2. For example, in certain embodiments, compounds of Formula II can effectively inhibit SARS-CoV-2 in human bronchial epithelial cells at sub-nanomolar and nanomolar concentrations. Thus, the compounds of the disclosure are particularly useful in treating COVID-19.

### Therapeutic Applications

One aspect of the disclosure provides a method of treating disease caused by SARS-CoV-2, commonly referred to as COVID-19. Such method includes administering to a subject in need thereof one or more compounds described herein.

In certain embodiments, the method comprises administering to a subject in need thereof a therapeutically effective amount of one or more compounds of Formula I or a pharmaceutically acceptable salt thereof (i.e., as otherwise described herein); or a pharmaceutical composition comprising one or more compounds of Formula I or a pharmaceutically acceptable salt thereof, and a pharmaceutically acceptable carrier, excipient, adjuvant, and/or diluent (i.e., as otherwise described herein). In certain such embodiments, the compound of Formula I is a carbamate derivative of a compound of Table 1. In certain such embodiments, the compound of Formula I is 4-bromo-2-((2-chloro-4-nitrophenyl)carbamoyl)phenyl piperazine-1-carboxylate; 4-chloro-2-((2-methoxy-4-nitrophenyl)carbamoyl)phenyl piperazine-1-carboxylate; 4-chloro-2-((4-nitrophenyl)carbamoyl)phenyl piperazine-1-carboxylate; or 4-bromo-2-((3-fluoro-4-(trifluoromethyl)phenyl)carbamoyl)phenyl piperazine-1-carboxylate. In certain such embodiments, the compound of Formula I is 4-chloro-2-((2-chloro-4-nitrophenyl)carbamoyl)phenyl 4-(trifluoromethyl)benzoate; 4-chloro-2-((2-chloro-4-nitrophenyl)carbamoyl)phenyl piperazine-1-carboxylate; 4-chloro-2-((2-chloro-4-nitrophenyl)carbamoyl)phenyl 2-methoxyacetate; 4-chloro-2-((2-chloro-4-nitrophenyl)carbamoyl)phenyl isopropyl carbonate; or 4-chloro-2-((2-chloro-4-nitrophenyl)carbamoyl)phenyl isobutyl carbonate.

In certain embodiments, the method comprises administering to a subject in need thereof a therapeutically effective amount of 4-chloro-2-((2-chloro-4-nitrophenyl)carbamoyl)phenyl 4-methylpiperazine-1-carboxylate; 4-chloro-2-((2-chloro-4-nitrophenyl)carbamoyl)phenyl octanoate; 4-chloro-2-((2-chloro-4-nitrophenyl)carbamoyl)phenyl acetate; 4-chloro-2-((2-chloro-4-nitrophenyl)carbamoyl)phenyl benzoate; 4-chloro-2-((2-chloro-4-nitrophenyl)carbamoyl)phenyl diethyl phosphate; or a pharmaceutically acceptable salt thereof; or a pharmaceutical composition comprising 4-chloro-2-((2-chloro-4-nitrophenyl)carbamoyl)phenyl 4-methylpiperazine-1-carboxylate; 4-chloro-2-((2-chloro-4-nitrophenyl)carbamoyl)phenyl octanoate; 4-chloro-2-((2-chloro-4-nitrophenyl)carbamoyl)phenyl acetate; 4-chloro-2-((2-chloro-4-nitrophenyl)carbamoyl)phenyl benzoate; 4-chloro-2-((2-chloro-4-nitrophenyl)carbamoyl)phenyl diethyl phosphate; or a pharmaceutically acceptable salt thereof, and a pharmaceutically acceptable carrier, excipient, adjuvant, and/or diluent.

In certain embodiments, the method comprises administering to a subject in need thereof a therapeutically effective amount of one or more compounds of Formula II or a pharmaceutically acceptable salt thereof (i.e., as otherwise described herein); or a pharmaceutical composition comprising one or more compounds of Formula II or a pharmaceutically acceptable salt thereof, and a pharmaceutically acceptable carrier, excipient, adjuvant, and/or diluent (i.e., as otherwise described herein). In certain such embodiments, the compound of Formula II is a compound of Table 2. In certain such embodiments, the compound of Formula II is 5-bromo-N-(2-chloro-4-nitrophenyl)-2-hydroxybenzamide; 5-chloro-N-(2-chloro-4-cyanophenyl)-2-hydroxybenzamide; 5-chloro-2-hydroxy-N-(4-(trifluoromethyl)phenyl)benzamide; N-(2-chloro-4-nitrophenyl)-1H-indole-7-carboxamide; 5-chloro-2-hydroxy-N-(2-methoxy-4-nitrophenyl)benzamide; 5-chloro-2-hydroxy-N-(4-nitrophenyl)benzamide; 5-bromo-N-(3-fluoro-4-(trifluoromethyl)phenyl)-2-hydroxybenzamide; or a pharmaceutically acceptable salt thereof.

### Compounds

As provided above, one aspect of the disclosure provides compounds of Formula I.

In certain embodiments as otherwise described herein, the compound of Formula I is not 4-chloro-2-((2-chloro-4-nitrophenyl)carbamoyl)phenyl 4-methylpiperazine-1-carboxylate.

In certain embodiments as otherwise described herein, X is —PO(R^{1A})(R^{2A}), and R^{1A} and R^{2A} are each independently C₁-C₆ alkoxy (e.g., C₂-C₄ alkoxy).

In certain embodiments as otherwise described herein X is —C(O)R^{1B}, and R^{1B} is aryl, optionally substituted with one or more (e.g., one) R¹⁴ independently selected from halogen and C₁-C₄ haloalkyl (e.g., —CF₃). In certain embodiments as otherwise described herein, X is —C(O)R^{1B}, and R^{1B} is C₁-C₁₂ alkyl (e.g., C₄-C₁₀ alkyl).

In certain embodiments as otherwise described herein, X is —C(O)NR^{1C}R^{2C}. In certain such embodiments, R^{1C} and R^{2C} are each independently hydrogen, C₁-C₆ alkyl, or amino(C₁-C₆ alkyl), or R^{1C} and R^{2C}, together with the atoms to which they are attached, form a 5- to 8-membered monocyclic heterocyclyl optionally substituted with one or more R¹⁵ selected from C₁-C₆ alkyl, —NH₂, —NH(C₁-C₆ alkyl), —N(C₁-C₆ alkyl)₂, —OH, C₁-C₆ alkoxy, cycloalkyl, or heterocycloalkyl; or R^{1C} and R^{2C}, together with the atoms to which they are attached, form a bicyclic heterocyclyl selected from a 5- to 8-membered monocyclic heterocyclyl bridged with 1-3 additional carbon atoms, and a 5- to 8-membered monocyclic heterocyclyl fused to a monocyclic cycloalkyl, monocyclic aryl, a monocyclic heterocycloalkyl, or a monocyclic heteroaryl.

In certain embodiments as otherwise described herein, X is —C(O)NR^{1C}R^{2C}, and R^{1C} and R^{2C}, together with the atoms to which they are attached, form a bicyclic heterocyclyl selected from a 5- to 8-membered monocyclic heterocyclyl bridged with 1-3 additional carbon atoms, and a 5- to 8-membered monocyclic heterocyclyl fused to a monocyclic cycloalkyl, monocyclic aryl, a monocyclic heterocycloalkyl, or a monocyclic heteroaryl. In certain such embodiments, the bicyclic heterocyclyl is a 5- to 8-membered (e.g., an 8-membered) monocyclic heterocyclyl bridged with 1-3 (e.g., 1) additional carbon atoms. In certain such embodiments, the bicyclic heterocyclic is a 5- to 8-membered monocyclic heterocyclyl fused to a monocyclic heterocyclyl or a monocyclic heteroaryl.

In certain embodiments as otherwise described herein, X is —C(O)NR^{1C}R^{2C}, and R^{1C} and R^{2C}, together with the atoms to which they are attached, form a 5- to 8-membered monocyclic heterocyclyl optionally substituted with one or more R¹⁵ selected from C₁-C₆ alkyl, —NH₂, —NH(C₁-C₆ alkyl), —N(C₁-C₆ alkyl)₂, —OH, C₁-C₆ alkoxy, cycloalkyl, or heterocycloalkyl. In certain such embodiments, R^{1C} and R^{2C}, together with the atoms to which they are attached, form a 5- to 6-membered monocyclic heterocyclyl optionally substituted with one R¹⁵ is selected from C₁-C₆ alkyl (e.g., —CH₃), C₁-C₆ alkoxy (e.g., —OCH₃), —OH, and —NH₂.

In certain embodiments as otherwise described herein, X is —C(O)NR^{1C}R^{2C}, and R^{1C} and R^{2C}, together with the atoms to which they are attached, form a 5- or 6-membered monocyclic heterocyclyl (e.g., a 6-membered monocyclic heterocyclyl).

For example, in certain embodiments as otherwise described herein, X is

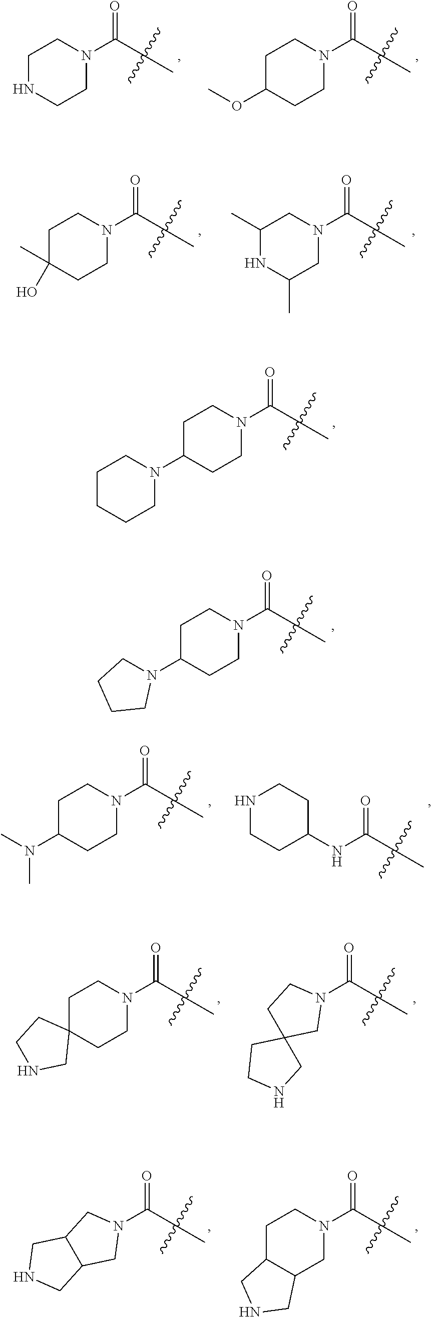

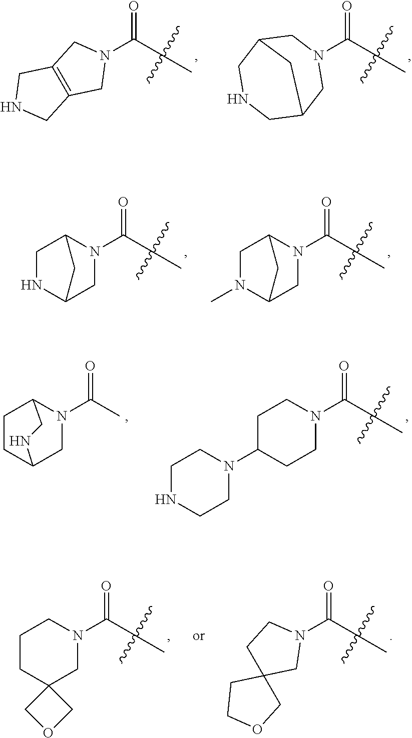

In certain such embodiments, R³, R⁴, and R⁶ are hydrogen, and Q is CR⁵ and R⁵ is halogen (e.g., —Cl or —Br). In certain such embodiments, Y is

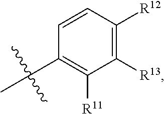

R¹¹ is halogen (e.g., —Cl), R¹² is —NO₂, and R¹³ is hydrogen.

In certain embodiments as otherwise described herein, the compound is of Formula I-A

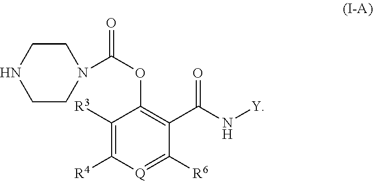

In certain embodiments as otherwise described herein, Q is CR⁵.

In certain embodiments as otherwise described herein, R³ and R⁴ are each independently hydrogen, halogen, or C₁-C₆ alkoxy, or R³ and R⁴, together with the atoms to which they are attached, form a 6-membered aryl optionally substituted with one or more R¹⁴ independently selected from halogen, —NO₂, and —CN. In certain embodiments as otherwise described herein, R³ and R⁴ are each independently hydrogen, halogen (e.g., —F, —Cl, or —Br), or C₁-C₆ alkoxy (e.g., —OCH₃). In certain embodiments as otherwise described herein, R³ and R⁴ are each independently hydrogen or halogen (e.g., —F, —Cl, or —Br). In certain embodiments as otherwise described herein, R³ is hydrogen and R⁴ is halogen (e.g., —F, —Cl, or —Br) or C₁-C₆ alkoxy (e.g., —OCH₃). In certain embodiments as otherwise described herein, R³ and R⁴ are each hydrogen.

In certain embodiments as otherwise described herein, the compound of Formula I is

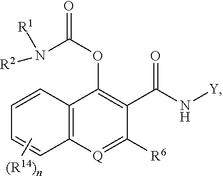

wherein
n is 0-4 (e.g., 0-2), and each R¹⁴ is independently selected from halogen, —NO₂, and —CN. In certain such embodiments, n is 1 and R¹⁴ is halogen (e.g., —F, —Cl, or —Br). In other such embodiments, n is 0.

In certain embodiments as otherwise described herein, Q is CR⁵, and R⁵ and R⁶ are each independently hydrogen, halogen, —NO₂, C₁-C₆ alkyl, —OH, —CO(C₁-C₆ alkyl), or aryl optionally substituted with one or more R¹⁴ independently selected from halogen, —NO₂, and —CN, or R⁵ and R⁶, together with the atoms to which they are attached, form a 6-membered aryl optionally substituted with one or more R¹⁴ independently selected from halogen, —NO₂, and —CN. In certain embodiments as otherwise described herein, Q is CR⁵, and R⁵ and R⁶ are each independently hydrogen, halogen (e.g., —F, —Cl, or —Br), —NO₂, C₁-C₆ alkyl (e.g., —CH₃), —OH, or —CO(C₁-C₆ alkyl) (e.g., —CO(CH₃)). In certain embodiments as otherwise described herein, Q is CR⁵, and R⁵ and R⁶ are each independently hydrogen, halogen (e.g., —F, —Cl, or —Br), C₁-C₆ alkyl (e.g., —CH₃), or —OH. In certain embodiments as otherwise described herein, Q is CR⁵, and R⁵ is aryl optionally substituted with one or more (e.g., two) R¹⁴ selected from halogen (e.g., —F), —NO₂, and —CN.

In certain embodiments as otherwise described herein, the compound of Formula I is

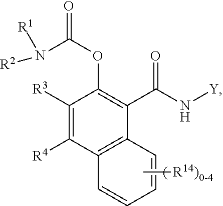

wherein
n is 0-4 (e.g., 0-2), and each R¹⁴ is independently selected from halogen, —NO₂, and —CN. In certain such embodiments, n is 1 and R¹⁴ is halogen (e.g., —F, —Cl, or —Br). In other such embodiments, n is 0.

In certain embodiments as otherwise described herein, R³ and R⁶ are hydrogen. In certain embodiments as otherwise described herein, R³, R⁴, and R⁶ are hydrogen, and Q is CR⁵, and R⁵ is halogen (e.g., —F, —Cl, or —Br). In certain embodiments as otherwise described herein, R⁶ is halogen (e.g., —Cl) or —OH.

In certain embodiments as otherwise described herein, R³ and R⁶ are hydrogen, R⁴ is hydrogen, halogen (e.g., —F, —Cl, or —Br), or C₁-C₆ alkoxy (e.g., —OCH₃), and Q is CR⁵, and R⁵ is hydrogen, halogen (e.g., —F, —Cl, or —Br), —NO₂, C₁-C₆ alkyl (e.g., —CH₃), —CO(C₁-C₆ alkyl) (e.g., —CO(CH₃)), or aryl optionally substituted with one or more (e.g., two) R¹⁴ independently selected from halogen (e.g., —F), —NO₂, and —CN, and at least one of R⁴ and R⁵ is not hydrogen. For example, in certain such embodiments, R⁴ is halogen (e.g., —Cl) or C₁-C₆ alkoxy (e.g., —OCH₃), and R⁵ is hydrogen. In another example, in certain such embodiments, R⁴ is hydrogen, and R⁵ is halogen (e.g., —F, —Cl, or —Br), —NO₂, C₁-C₆ alkyl (e.g., —CH₃), —CO(C₁-C₆ alkyl) (e.g., —CO(CH₃)), or aryl optionally substituted with one or more (e.g., two) R¹⁴ independently selected from halogen (e.g., —F), —NO₂, and —CN.

In certain embodiments as otherwise described herein,

### Y is

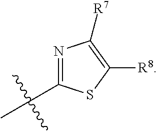

In certain such embodiments, R⁷ and R⁸ are each independently hydrogen, —NO₂, or C₁-C₆ alkyl. In certain embodiments as otherwise described herein, R⁷ and R⁸ are each independently hydrogen or C₁-C₆ alkyl (e.g., C₂-C₄ alkyl). In certain embodiments as otherwise described herein, R⁷ and R⁸ are each independently hydrogen or —NO₂. For example, in certain such embodiments, R⁷ is hydrogen, and R⁸ is C₂-C₄ alkyl (e.g., —CH(CH₃)₂). In another example, in certain such embodiments, R⁷ is hydrogen, and R⁸ is —NO₂.

In certain embodiments as otherwise described herein,

### Y is

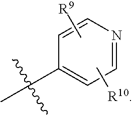

In certain such embodiments, R⁹ and R¹⁰ are each independently hydrogen, —NO₂, or C₁-C₆ alkyl. In certain embodiments as otherwise described herein, R⁹ and R¹⁰ are each independently hydrogen or halogen (e.g., —F, —Cl, or —Br). For example, in certain such embodiments, R⁹ is halogen (e.g., —Cl) and R¹⁰ is hydrogen.

In certain embodiments as otherwise described herein,

### Y is

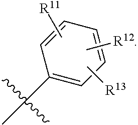

In certain such embodiments, R¹¹, R¹², and R¹³ are each independently hydrogen, halogen, —NO₂, —CN, C₁-C₆ alkyl, C₁-C₆ haloalkyl, —NHCO(C₁-C₆ alkyl), C₁-C₆ alkoxy, —CON(C₁-C₆ alkyl)₂, or —S(O)₀₋2NH₂, or R¹¹ and R¹³, together with the atoms to which they are attached, form a 6-membered aryl optionally substituted with one or more R¹⁴ independently selected from halogen, —NO₂, and —CN. In certain such embodiments, R¹¹ is halogen (e.g., —F or —Cl), R¹² is —NO₂, —CN, halogen (e.g., —F), or C₁-C₆ haloalkyl (e.g., CF₃), and R¹³ is hydrogen.

For example, in certain embodiments as otherwise described herein,

### Y is

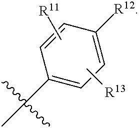

In certain such embodiments, R¹¹ is hydrogen, halogen (e.g., —F, —Cl, or —Br), or C₁-C₆ alkoxy, R¹² is hydrogen, halogen (e.g., —F, —Cl, or —Br), —NO₂, —CN, C₁-C₆ haloalkyl (e.g., CF₃), or —S(O)₀₋₂NH₂, R¹³ is hydrogen, halogen (e.g., —F, —Cl, or —Br), —NO₂, or C₁-C₆ alkoxy (e.g., —OCH₃), or R¹¹ and R¹³, together with the atoms to which they are attached, form a 6-membered aryl, and at least one of R¹¹, R¹², and R¹³ is not hydrogen. In certain such embodiments, R¹¹ is halogen (e.g., —F or —Cl), R¹² is —NO₂, —CN, halogen (e.g., —F), or C₁-C₆ haloalkyl (e.g., CF₃), and R¹³ is hydrogen. In certain embodiments, R¹¹ is C₁-C₆ alkoxy, R¹² is —NO₂, and R¹³ is hydrogen. In certain embodiments, R¹¹ is hydrogen, R¹² is —NO₂, and R¹³ is hydrogen. In certain embodiments, R¹¹ is hydrogen, R¹² is —CF³, and R¹³ is halogen.

In another example, in certain embodiments as otherwise described herein,

### Y is

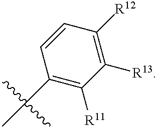

In certain such embodiments, R¹¹ and R¹³ are each independently hydrogen, halogen (e.g., —F, —Cl, or —Br), or C₁-C₆ alkoxy, R¹² is hydrogen, halogen (e.g., —F, —Cl, or —Br), —NO₂, —CN, C₁-C₆ haloalkyl (e.g., CF₃), or —S(O)₀₋₂NH₂, and at least one of R¹¹, R¹², and R¹³ is not hydrogen. In certain such embodiments, R¹¹ is halogen (e.g., —F or —Cl), R¹² is —NO₂, —CN, halogen (e.g., —F), or C₁-C₆ haloalkyl (e.g., CF₃), and R¹³ is hydrogen. In other such embodiments, R¹¹ is hydrogen, R¹² is —NO₂, —CN, halogen (e.g., —F), or C₁-C₆ haloalkyl (e.g., CF₃), and R¹³ is halogen (e.g., —F or —Cl). In certain embodiments, R¹¹ is C₁-C₆ alkoxy, R¹² is —NO₂, and R¹³ is hydrogen. In certain embodiments, R¹¹ is hydrogen, R¹² is —NO₂, and R¹³ is hydrogen. In certain embodiments, R¹¹ is hydrogen, R¹² is —CF³, and R¹³ is hydrogen or halogen. In certain embodiments, R¹¹ is halogen, R¹² is —CN, and R¹³ is hydrogen.

In another example, in certain embodiments as otherwise described herein,

### Y is

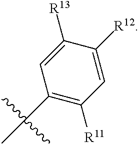

In certain such embodiments, R¹¹ is hydrogen or halogen (e.g., —F, —Cl, or —Br), R¹² is hydrogen, halogen (e.g., —F, —Cl, or —Br), —NO₂, —CN, C₁-C₆ haloalkyl (e.g., CF₃), or —S(O)₀₋₂NH₂, R¹³ is hydrogen, halogen (e.g., —F, —Cl, or —Br), or NO₂, and at least one of R¹¹, R¹², and R¹³ is not hydrogen. In certain such embodiments, R¹¹ and R¹² are each independently hydrogen or halogen (e.g., —F, —Cl, or —Br), and R¹³ is —NO₂.

In certain desirable embodiments as otherwise described herein, X is —C(O)NR^{1C}R^{2C}, and R^{1C} and R^{2C} are each independently hydrogen, C₁-C₆ alkyl, or amino(C₁-C₆ alkyl), or R^{1C} and R^{2C}, together with the atoms to which they are attached, form a 5- or 6-membered heterocyclyl; R³ and R⁴ are each independently hydrogen, halogen (e.g., —F, —Cl, or —Br), or C₁-C₆ alkoxy (e.g., —OCH₃); Q is CR⁵, and R⁵ and R⁶ are each independently hydrogen, halogen (e.g., —F, —Cl, or —Br), —NO₂, C₁-C₆ alkyl (e.g., —CH₃), —OH, or —CO(C₁-C₆ alkyl) (e.g., —CO(CH₃)); and

### Y is

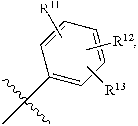

wherein R¹¹, R¹², and R¹³ are each independently hydrogen, halogen, —NO₂, —CN, C₁-C₆ alkyl, C₁-C₆ haloalkyl, —NHCO(C₁-C₆ alkyl), C₁-C₆ alkoxy, —CON(C₁-C₆ alkyl)₂, or —S(O)₀₋₂NH₂, or R¹¹ and R¹³, together with the atoms to which they are attached, form a 6-membered aryl optionally substituted with one or more R¹⁴ independently selected from halogen, —NO₂, and —CN.

In certain such embodiments, R^{1C} and R^{2C}, together with the atoms to which they are attached, form a 5- or 6-membered heterocyclyl (e.g., a 6-membered heterocyclyl). In certain such embodiments, R³ and R⁴ are each independently hydrogen or halogen (e.g., —F, —Cl, or —Br). In certain such embodiments, R³ is hydrogen and R⁴ is halogen (e.g., —F, —Cl, or —Br) or C₁-C₆ alkoxy (e.g., —OCH₃). In certain such embodiments, R³ and R⁴ are each hydrogen. In certain such embodiments, R⁵ and R⁶ are each independently hydrogen, halogen (e.g., —F, —Cl, or —Br), C₁-C₆ alkyl (e.g., —CH₃), or —OH. In certain such embodiments, Q is CR⁵, and R⁵ is aryl optionally substituted with one or more (e.g., two) R¹⁴ selected from halogen (e.g., —F), —NO₂, and —CN. In certain such embodiments, R⁶ is halogen (e.g., —Cl) or —OH. In certain such embodiments, R¹¹ is halogen (e.g., —F or —Cl), R¹² is —NO₂, —CN, halogen (e.g., —F), or C₁-C₆ haloalkyl (e.g., CF₃), and R¹³ is hydrogen.

In certain desirable embodiments as otherwise described herein, X is —C(O)NR^{1C}R^{2C}, and R^{1C} and R^{2C} are each independently hydrogen, C₁-C₆ alkyl, or amino(C₁-C₆ alkyl), or R^{1C} and R^{2C}, together with the atoms to which they are attached, form a 5- or 6-membered heterocyclyl; R³ and R⁶ are hydrogen, R⁴ is hydrogen, halogen (e.g., —F, —Cl, or —Br), or C₁-C₆ alkoxy (e.g., —OCH₃), and Q is CR⁵, and R⁵ is hydrogen, halogen (e.g., —F, —Cl, or —Br), —NO₂, C₁-C₆ alkyl (e.g., —CH₃), —CO(C₁-C₆ alkyl) (e.g., —CO(CH₃)), or aryl optionally substituted with one or more (e.g., two) R¹⁴ independently selected from halogen (e.g., —F), —NO₂, and —CN, and at least one or R⁴ and R⁵ is not hydrogen; and

### Y is

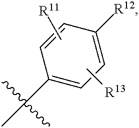

wherein R¹¹ is hydrogen or halogen (e.g., —F, —Cl, or —Br), R¹² is hydrogen, halogen (e.g., —F, —Cl, or —Br), —NO₂, —CN, C₁-C₆ haloalkyl (e.g., CF₃), or —S(O)₀₋₂NH₂, R¹³ is hydrogen, halogen (e.g., —F, —Cl, or —Br), —NO₂, or C₁-C₆ alkoxy (e.g., —OCH₃), or R¹¹ and R¹³, together with the atoms to which they are attached, form a 6-membered aryl, and at least one of R¹¹, R¹², and R¹³ is not hydrogen.

In certain such embodiments, R^{1C} and R^{2C}, together with the atoms to which they are attached, form a 5- or 6-membered heterocyclyl (e.g., a 6-membered heterocyclyl). In certain such embodiments, R⁴ is halogen (e.g., —Cl) or C₁-C₆ alkoxy (e.g., —OCH₃), and Q is CR⁵, and R⁵ is hydrogen. In certain such embodiments, R⁴ is hydrogen, and R⁵ is halogen (e.g., —F, —Cl, or —Br), —NO₂, C₁-C₆ alkyl (e.g., —CH₃), —CO(C₁-C₆ alkyl) (e.g., —CO(CH₃)), or aryl optionally substituted with one or more (e.g., two) R¹⁴ independently selected from halogen (e.g., —F), —NO₂, and —CN. In certain such embodiments, R¹¹ is halogen (e.g., —F or —Cl), R¹² is —NO₂, —CN, halogen (e.g., —F), or C₁-C₆ haloalkyl (e.g., CF₃), and R¹³ is hydrogen. In certain such embodiments, R¹¹ and R¹² are each independently hydrogen or halogen (e.g., —F, —Cl, or —Br), and R¹³ is —NO₂.

In certain desirable embodiments as otherwise described herein, X is —C(O)NR^{1C}R^{2C}; and

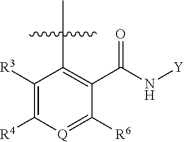

is 4-chloro-2-((2-chloro-4-nitrophenyl)carbamoyl)phenyl; 2-((2-chloro-4-nitrophenyl)carbamoyl)-4-fluorophenyl; 4-bromo-2-((2-chloro-4-nitrophenyl)carbamoyl)phenyl; 5-chloro-2-((2-chloro-4-nitrophenyl)carbamoyl)phenyl; 2-((2-chloro-4-nitrophenyl)carbamoyl)-5-methoxyphenyl; 2-((2-chloro-4-nitrophenyl)carbamoyl)-4-methoxyphenyl; 2-((2-chloro-4-nitrophenyl)carbamoyl)-4-methylphenyl; 2-((2-chloro-4-nitrophenyl)carbamoyl)-3-hydroxyphenyl; 2-((2-chloro-4-nitrophenyl)carbamoyl)-4-nitrophenyl; 4-acetyl-2-((2-chloro-4-nitrophenyl)carbamoyl)phenyl; 2-((2-chloro-4-nitrophenyl)carbamoyl)naphthalen-1-yl; 1-((2-chloro-4-nitrophenyl)carbamoyl)naphthalen-2-yl; 3-((2-chloro-4-nitrophenyl)carbamoyl)-2′,4′-difluoro-[1,1′-biphenyl]-4-yl; 4-chloro-2-((2-chlorophenyl)carbamoyl)phenyl; 4-chloro-2-((4-nitrophenyl)carbamoyl)phenyl; 4-chloro-2-((2,6-dichloro-4-nitrophenyl)carbamoyl)phenyl; 4-chloro-2-((3-chloro-4-nitrophenyl)carbamoyl)phenyl; 4-chloro-2-((2-methoxy-4-nitrophenyl)carbamoyl)phenyl; 4-chloro-2-((5-isopropylthiazol-2-yl)carbamoyl)phenyl; 4-chloro-2-((5-nitrothiazol-2-yl)carbamoyl)phenyl; 4-chloro-2-((3-chloropyridin-4-yl)carbamoyl)phenyl; 2-((4-amino-2-chlorophenyl)carbamoyl)-4-chlorophenyl; 4-chloro-2-((2-chloro-4-cyanophenyl)carbamoyl)phenyl; 4-chloro-2-((2-chloro-4-fluorophenyl)carbamoyl)phenyl; 4-chloro-2-((2,4-difluorophenyl)carbamoyl)phenyl; 4-chloro-2-((3-chloro-4-fluorophenyl)carbamoyl)phenyl; 4-chloro-2-((4-fluorophenyl)carbamoyl)phenyl; 4-chloro-2-((2,6-dichloro-4-fluorophenyl)carbamoyl)phenyl; 4-chloro-2-((4-sulfamoylphenyl)carbamoyl)phenyl; 4-chloro-2-((4-(trifluoromethyl)phenyl)carbamoyl)phenyl; 4-chloro-2-((2-chloro-4-(trifluoromethyl)phenyl)carbamoyl)phenyl; 4-chloro-2-((2-(trifluoromethyl)phenyl)carbamoyl)phenyl; 2-((4-acetamidophenyl)carbamoyl)-4-chlorophenyl; 4-chloro-2-((3-(dimethylcarbamoyl)phenyl)carbamoyl)phenyl; 4-chloro-2-((2,4-dichlorophenyl)carbamoyl)phenyl; 4-chloro-2-((4-chlorophenyl)carbamoyl)phenyl; 4-chloro-2-((4-chloro-2-fluoro-5-nitrophenyl)carbamoyl)phenyl; 4-chloro-2-((2,6-dichlorophenyl)carbamoyl)phenyl; 4-chloro-2-((2,6-difluorophenyl)carbamoyl)phenyl; 4-chloro-2-((4-methoxyphenyl)carbamoyl)phenyl; 4-chloro-2-((3-methoxyphenyl)carbamoyl)phenyl; 4-chloro-2-((2,3-dimethylphenyl)carbamoyl)phenyl; 4-chloro-2-((2-chloro-5-methoxyphenyl)carbamoyl)phenyl; 4-chloro-2-(p-tolylcarbamoyl)phenyl; 4-chloro-2-((2-nitrophenyl)carbamoyl)phenyl; 4-chloro-2-((6-nitronaphthalen-2-yl)carbamoyl)phenyl; 4-chloro-2-((4-nitronaphthalen-1-yl)carbamoyl)phenyl; 4-bromo-2-((4-(trifluoromethyl)phenyl)carbamoyl)phenyl; 4-bromo-2-((3-chloro-4-(trifluoromethyl)phenyl)carbamoyl)phenyl; 4-bromo-2-((3-fluoro-4-(trifluoromethyl)phenyl)carbamoyl)phenyl; 4-bromo-2-((2-chloro-4-(trifluoromethyl)phenyl)carbamoyl)phenyl; 4-bromo-2-((2-methoxy-4-nitrophenyl)carbamoyl)phenyl; 4-bromo-2-((3-chloro-4-nitrophenyl)carbamoyl)phenyl; 2-((2-chloro-4-(trifluoromethyl)phenyl)carbamoyl)naphthalen-1-yl; 2-((2-chloro-4-cyanophenyl)carbamoyl)naphthalen-1-yl; 3-((2-chloro-4-(trifluoromethyl)phenyl)carbamoyl)-2′,4′-difluoro-[1,1′-biphenyl]-4-yl; 3-((2-chloro-4-cyanophenyl)carbamoyl)-2′,4′-difluoro-[1,1′-biphenyl]-4-yl; or 3-((2-chloro-4-nitrophenyl)carbamoyl)quinolin-4-yl.

In certain such embodiments, R^{1C} and R^{2C} are each independently hydrogen, C₁-C₆ alkyl, or amino(C₁-C₆ alkyl), or R^{1C} and R^{2C}, together with the atoms to which they are attached, form a 5- or 6-membered heterocyclyl. In certain such embodiments, R^{1C} and R^{2C}, together with the atoms to which they are attached, form a 5- or 6-membered heterocyclyl (e.g., a 6-membered heterocyclyl). In certain such embodiments, R^{1C} and R^{2C}, together with the atoms to which they are attached, form a piperazinyl.

In certain such embodiments, X is

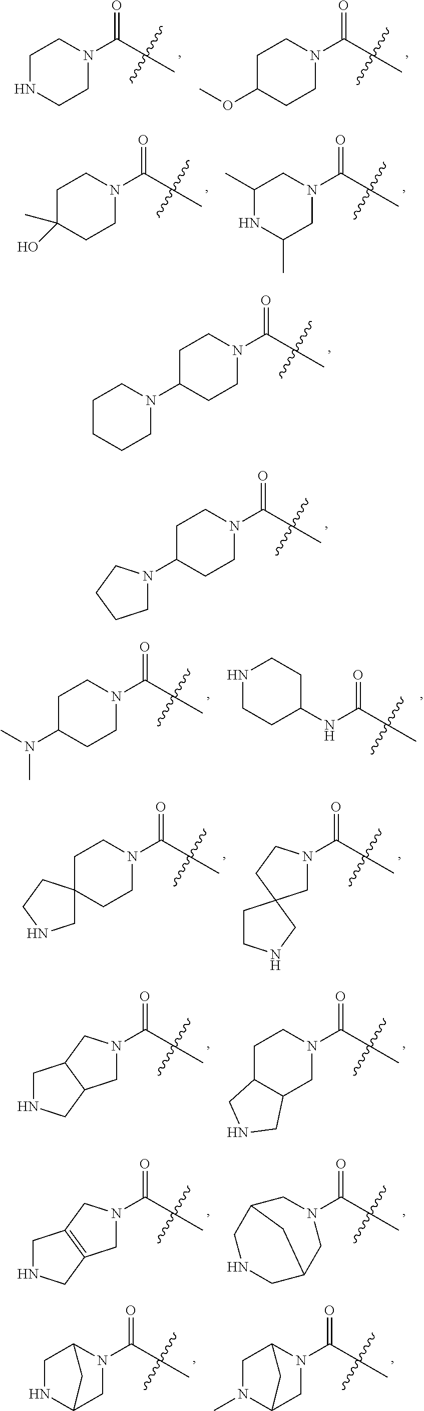

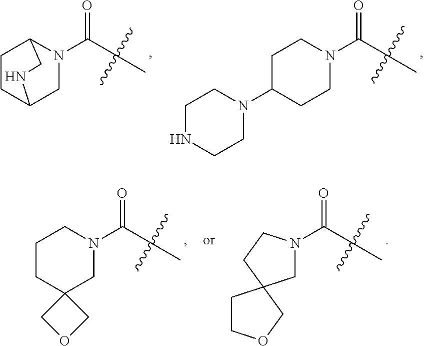 [SMILES: *C(=O)N1CC2CCC1CN2 ; *C(=O)N1CCC(N2CCNCC2)CC1 ; *C(=O)N1CCCC2(COC2)C1 ; *C(=O)N1CCC2(CCOC2)C1]

Particularly useful compounds of Formula I are provided in Table 1.

TABLE 1

| No. | Structure | Compound |
|---|---|---|
| P1 | 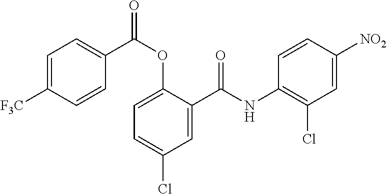 [SMILES: O=C(Oc1ccc(Cl)cc1C(=O)Nc1ccc([N+](=O)[O-])cc1Cl)c1ccc(C(F)(F)F)cc1] | 4-chloro-2-((2-chloro-4- nitrophenyl)carbamoyl)phenyl 4- (trifluoromethyl)benzoate |
| P2 | 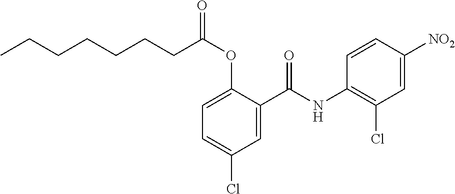 [SMILES: CCCCCCCC(=O)Oc1ccc(Cl)cc1C(=O)Nc1ccc([N+](=O)[O-])cc1Cl] | 4-chloro-2-((2-chloro-4- nitrophenyl)carbamoyl)phenyl octanoate |
| P3 | 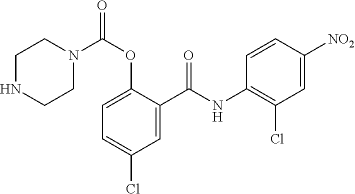 [SMILES: O=C(Nc1ccc([N+](=O)[O-])cc1Cl)c1cc(Cl)ccc1OC(=O)N1CCNCC1] | 4-chloro-2-((2-chloro-4- nitrophenyl)carbamoyl)phenyl piperazine-1-carboxylate |
| P4 | 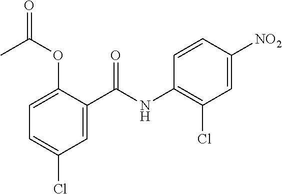 [SMILES: CC(=O)Oc1ccc(Cl)cc1C(=O)Nc1ccc([N+](=O)[O-])cc1Cl] | 4-chloro-2-((2-chloro-4- nitrophenyl)carbamoyl)phenyl acetate |
| P5 | 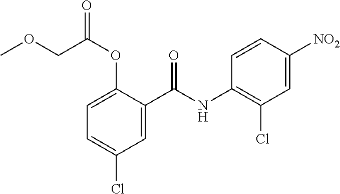 [SMILES: COCC(=O)Oc1ccc(Cl)cc1C(=O)Nc1ccc([N+](=O)[O-])cc1Cl] | 4-chloro-2-((2-chloro-4- nitrophenyl)carbamoyl)phenyl 2- methoxyacetate |
| P6 | 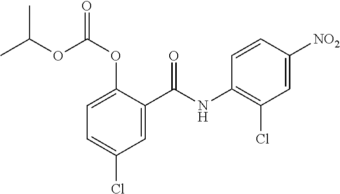 [SMILES: CC(C)OC(=O)Oc1ccc(Cl)cc1C(=O)Nc1ccc([N+](=O)[O-])cc1Cl] | 4-chloro-2-((2-chloro-4- nitrophenyl)carbamoyl)phenyl isopropyl carbonate |
| P7 | 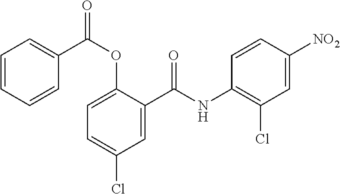 [SMILES: O=C(Oc1ccc(Cl)cc1C(=O)Nc1ccc([N+](=O)[O-])cc1Cl)c1ccccc1] | 4-chloro-2-((2-chloro-4- nitrophenyl)carbamoyl)phenyl benzoate |
| P8 | 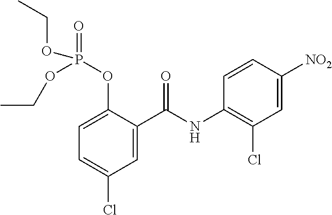 [SMILES: CCOP(=O)(OCC)Oc1ccc(Cl)cc1C(=O)Nc1ccc([N+](=O)[O-])cc1Cl] | 4-chloro-2-((2-chloro-4- nitrophenyl)carbamoyl)phenyl diethyl phosphate |
| P9 | 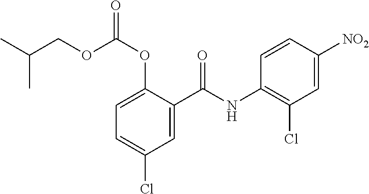 [SMILES: CC(C)COC(=O)Oc1ccc(Cl)cc1C(=O)Nc1ccc([N+](=O)[O-])cc1Cl] | 4-chloro-2-((2-chloro-4- nitrophenyl)carbamoyl)phenyl isobutyl carbonate |

In certain embodiments, the compound of Formula I is selected from P1, P3, P5, P6, and P9 listed in Table I.

In certain desirable embodiments as otherwise described herein, the compound is 4-bromo-2-((2-chloro-4-nitrophenyl)carbamoyl)phenyl piperazine-1-carboxylate; 4-chloro-2-((2-methoxy-4-nitrophenyl)carbamoyl)phenyl piperazine-1-carboxylate; 4-chloro-2-((4-nitrophenyl)carbamoyl)phenyl piperazine-1-carboxylate; 4-bromo-2-((3-fluoro-4-(trifluoromethyl)phenyl)carbamoyl)phenyl piperazine-1-carboxylate; or a pharmaceutically acceptable salt thereof.

In another aspect, the disclosure provides compounds of Formula II as provided above.

In certain embodiments as otherwise described herein, X is —OR²¹, and R²¹ is hydrogen, C₁-C₆ alkyl, amino(C₁-C₆ alkyl), or heterocycloalkyl. In certain embodiments as otherwise described herein, X is —OR²¹, and R²¹ is hydrogen, C₁-C₆ alkyl, or amino(C₁-C₆ alkyl). In certain embodiments as otherwise described herein, X is —OR²¹, and R²¹ is hydrogen or C₁-C₆ alkyl. In certain embodiments as otherwise described herein, R²¹ is C₁-C₄ alkyl (e.g., —CH₃). In certain embodiments as otherwise described herein, R²¹ is amino(C₁-C₄ alkyl) (e.g., —CH₂CH₂NH₂). In certain embodiments as otherwise described herein, R²¹ is heterocycloalkyl (e.g., 4-piperidinyl). In certain embodiments as otherwise described herein, R²¹ is hydrogen (i.e., X is —OH).

In certain embodiments as otherwise described herein, X is —NHR²², and R²² is hydrogen, —CO(C₁-C₆ alkyl), —S(O)₀₋₂(C₁-C₆ alkyl), or —S(O)₀₋₂(aryl). In certain embodiments as otherwise described herein, R²² is hydrogen. In certain embodiments as otherwise described herein, R²² is —CO(C₁-C₄ alkyl) (e.g., —CO(CH₃)). In certain embodiments as otherwise described herein, R²² is —S(O)₀₋₂(C₁-C₄ alkyl) (e.g., —SO₂(CH₃)). In certain embodiments as otherwise described herein, R²² is —S(O)₀₋₂(aryl) (e.g., —SO₂(phenyl)).

In certain embodiments as otherwise described herein, X is —NHR²², and R²² and R²³, together with the atoms to which they are attached, form a 5-membered heteroaryl. For example, in certain such embodiments, the compound is of Formula II-A

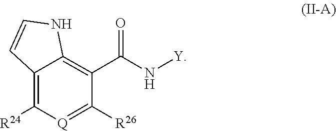

In certain embodiments as otherwise described herein, Q is CR²⁵.

In certain embodiments as otherwise described herein, R²³ and R²⁴ are each independently hydrogen, halogen, or C₁-C₆ alkoxy, or R²³ and R²⁴, together with the atoms to which they are attached, form a 6-membered aryl optionally substituted with one or more R³⁴ independently selected from halogen, —NO₂, and —CN. In certain embodiments as otherwise described herein, R²³ and R²⁴ are each independently hydrogen, halogen (e.g., —F, —Cl, or —Br), or C₁-C₆ alkoxy (e.g., —OCH₃). In certain embodiments as otherwise described herein, R²³ and R²⁴ are each independently hydrogen or halogen (e.g., —F, —Cl, or —Br). In certain embodiments as otherwise described herein, R²³ is hydrogen and R²⁴ is halogen (e.g., —F, —Cl, or —Br) or C₁-C₆ alkoxy (e.g., —OCH₃). In certain embodiments as otherwise described herein, R²³ and R²⁴ are each hydrogen.

In certain embodiments as otherwise described herein, the compound of Formula II is

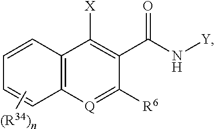

wherein
n is 0-4 (e.g., 0-2), and each R³⁴ is independently selected from halogen, —NO₂, and —CN. In certain such embodiments, n is 1 and R³⁴ is halogen (e.g., —F, —Cl, or —Br). In other such embodiments, n is 0.

In certain embodiments as otherwise described herein, Q is CR²⁵, and R²⁵ and R²⁶ are each independently hydrogen, halogen, —NO₂, C₁-C₆ alkyl, —OH, —CO(C₁-C₆ alkyl), or aryl optionally substituted with one or more R³⁴ independently selected from halogen, —NO₂, and —CN, or R²⁵ and R²⁶, together with the atoms to which they are attached, form a 6-membered aryl optionally substituted with one or more R³⁴ independently selected from halogen, —NO₂, and —CN. In certain embodiments as otherwise described herein, Q is CR²⁵, and R²⁵ and R²⁶ are each independently hydrogen, halogen (e.g., —F, —Cl, or —Br), —NO₂, C₁-C₆ alkyl (e.g., —CH₃), —OH, or —CO(C₁-C₆ alkyl) (e.g., —CO(CH₃)). In certain embodiments as otherwise described herein, Q is CR²⁵, and R²⁵ and R²⁶ are each independently hydrogen, halogen (e.g., —F, —Cl, or —Br), C₁-C₆ alkyl (e.g., —CH₃), or —OH. In certain embodiments as otherwise described herein, Q is CR²⁵, and R²⁵ is aryl optionally substituted with one or more (e.g., two) R³⁴ selected from halogen (e.g., —F), —NO₂, and —CN.

In certain embodiments as otherwise described herein, the compound of Formula II is

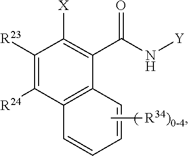

wherein
n is 0-4 (e.g., 0-2), and each R³⁴ is independently selected from halogen, —NO₂, and —CN. In certain such embodiments, n is 1 and R³⁴ is halogen (e.g., —F, —Cl, or —Br). In other such embodiments, n is 0.

In certain embodiments as otherwise described herein, R²³ and R²⁶ are hydrogen. In certain embodiments as otherwise described herein, R²³, R²⁴, and R²⁶ are hydrogen, and Q is CR²⁵, and R²⁵ is halogen (e.g., —F, —Cl, or —Br). In certain embodiments as otherwise described herein, R²⁶ is halogen (e.g., —Cl) or —OH.

In certain embodiments as otherwise described herein, R²³ and R²⁶ are hydrogen, R²⁴ is hydrogen, halogen (e.g., —F, —Cl, or —Br), or C₁-C₆ alkoxy (e.g., —OCH₃), and Q is CR²⁵, and R²⁵ is hydrogen, halogen (e.g., —F, —Cl, or —Br), —NO₂, C₁-C₆ alkyl (e.g., —CH₃), —CO(C₁-C₆ alkyl) (e.g., —CO(CH₃)), or aryl optionally substituted with one or more (e.g., two) R³⁴ independently selected from halogen (e.g., —F), —NO₂, and —CN, and at least one or R²⁴ and R²⁵ is not hydrogen. For example, in certain such embodiments, R²⁴ is halogen (e.g., —Cl) or C₁-C₆ alkoxy (e.g., —OCH₃), and R²⁵ is hydrogen. In another example, in certain such embodiments, R²⁴ is hydrogen, and R²⁵ is halogen (e.g., —F, —Cl, or —Br), —NO₂, C₁-C₆ alkyl (e.g., —CH₃), —CO(C₁-C₆ alkyl) (e.g., —CO(CH₃)), or aryl optionally substituted with one or more (e.g., two) R³⁴ independently selected from halogen (e.g., —F), —NO₂, and —CN.

In certain embodiments as otherwise described herein,

### Y is

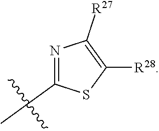

In certain such embodiments, R²⁷ and R²⁸ are each independently hydrogen, —NO₂, or C₁-C₆ alkyl. In certain embodiments as otherwise described herein, R²⁷ and R²⁸ are each independently hydrogen or C₁-C₆ alkyl (e.g., C₂-C₄ alkyl). In certain embodiments as otherwise described herein, R²⁷ and R²⁸ are each independently hydrogen or —NO₂. For example, in certain such embodiments, R²⁷ is hydrogen, and R²⁸ is C₂-C₄ alkyl (e.g., —CH(CH₃)₂). In another example, in certain such embodiments, R²⁷ is hydrogen, and R²⁸ is —NO₂.

In certain embodiments as otherwise described herein,

### Y is

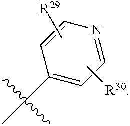

In certain such embodiments, R²⁹ and R³⁰ are each independently hydrogen, —NO₂, or C₁-C₆ alkyl. In certain embodiments as otherwise described herein, R²⁹ and R³⁰ are each independently hydrogen or halogen (e.g., —F, —Cl, or —Br). For example, in certain such embodiments, R²⁹ is halogen (e.g., —Cl) and R³⁰ is hydrogen.

In certain embodiments as otherwise described herein,

### Y is

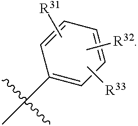

In certain such embodiments, R³¹, R³², and R³³ are each independently hydrogen, halogen, —NO₂, —CN, C₁-C₆ alkyl, C₁-C₆ haloalkyl, —NHCO(C₁-C₆ alkyl), C₁-C₆ alkoxy, —CON(C₁-C₆ alkyl)₂, or —S(O)₀₋₂NH₂, or R³¹ and R³³, together with the atoms to which they are attached, form a 6-membered aryl optionally substituted with one or more R³⁴ independently selected from halogen, —NO₂, and —CN. In certain such embodiments, R³¹ is halogen (e.g., —F or —Cl), R³² is —NO₂, —CN, halogen (e.g., —F), or C₁-C₆ haloalkyl (e.g., CF₃), and R³³ is hydrogen.

For example, in certain embodiments as otherwise described herein,

### Y is

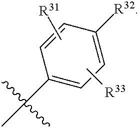

In certain such embodiments, R³¹ is hydrogen, halogen (e.g., —F, —Cl, or —Br), or C₁-C₆ alkoxy, R³² is hydrogen, halogen (e.g., —F, —Cl, or —Br), —NO₂, —CN, C₁-C₆ haloalkyl (e.g., CF₃), or —S(O)₀₋₂NH₂, R³³ is hydrogen, halogen (e.g., —F, —Cl, or —Br), —NO₂, or C₁-C₆ alkoxy (e.g., —OCH₃), or R³¹ and R³³, together with the atoms to which they are attached, form a 6-membered aryl, and at least one of R³¹, R³², and R³³ is not hydrogen. In certain such embodiments, R³¹ is halogen (e.g., —F or —Cl), R³² is —NO₂, —CN, halogen (e.g., —F), or C₁-C₆ haloalkyl (e.g., CF₃), and R³³ is hydrogen. In certain embodiments, R³¹ is C₁-C₆ alkoxy, R³² is —NO₂, and R³³ is hydrogen. In certain embodiments, R³¹ is hydrogen, R³² is —NO₂, and R³³ is hydrogen. In certain embodiments, R³¹ is hydrogen, R³² is —CF³, and R³³ is hydrogen or halogen. In certain embodiments, wherein R³¹ is halogen, R³² is —CN, and R³³ is hydrogen.

In another example, in certain embodiments as otherwise described herein,

### Y is

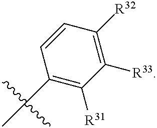

In certain such embodiments, R³¹ and R³³ are each independently hydrogen, halogen (e.g., —F, —Cl, or —Br), or C₁-C₆ alkoxy, R³² is hydrogen, halogen (e.g., —F, —Cl, or —Br), —NO₂, —CN, C₁-C₆ haloalkyl (e.g., CF₃), or —S(O)₀₋₂NH₂, and at least one of R³¹, R³², and R³³ is not hydrogen. In certain such embodiments, R³¹ is halogen (e.g., —F or —Cl), R³² is —NO₂, —CN, halogen (e.g., —F), or C₁-C₆ haloalkyl (e.g., CF₃), and R³³ is hydrogen. In other such embodiments, R³¹ is hydrogen, R³² is —NO₂, —CN, halogen (e.g., —F), or C₁-C₆ haloalkyl (e.g., CF₃), and R³³ is halogen (e.g., —F or —Cl). In certain embodiments, R³¹ is C₁-C₆ alkoxy, R³² is —NO₂, and R³³ is hydrogen. In certain embodiments, R³¹ is hydrogen, R³² is —NO₂, and R³³ is hydrogen. In certain embodiments, R³¹ is hydrogen, R³² is —CF³, and R³³ is halogen.

In another example, in certain embodiments as otherwise described herein,

### Y is

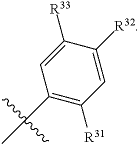

In certain such embodiments, R³¹ is hydrogen or halogen (e.g., —F, —Cl, or —Br), R³² is hydrogen, halogen (e.g., —F, —Cl, or —Br), —NO₂, —CN, C₁-C₆ haloalkyl (e.g., CF₃), or —S(O)₀₋₂NH₂, R³³ is hydrogen, halogen (e.g., —F, —Cl, or —Br), or NO₂, and at least one of R³¹, R³², and R³³ is not hydrogen. In certain such embodiments, R³¹ and R³² are each independently hydrogen or halogen (e.g., —F, —Cl, or —Br), and R³³ is —NO₂.

In certain desirable embodiments as otherwise described herein, X is —OR²¹; R²¹ is hydrogen, C₁-C₆ alkyl (e.g., C₁-C₄ alkyl), amino(C₁-C₆ alkyl) (e.g., amino(C₁-C₄ alkyl)), or heterocycloalkyl (e.g., piperidinyl); R²³ and R²⁴ are each independently hydrogen, halogen (e.g., —F, —Cl, or —Br), or C₁-C₆ alkoxy (e.g., —OCH₃); R²⁵ and R²⁶ are each independently hydrogen, halogen (e.g., —F, —Cl, or —Br), —NO₂, C₁-C₆ alkyl (e.g., —CH₃), —OH, or —CO(C₁-C₆ alkyl) (e.g., —CO(CH₃)); and

### Y is

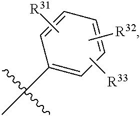

wherein R³¹, R³², and R³³ are each independently hydrogen, halogen, —NO₂, —CN, C₁-C₆ alkyl, C₁-C₆ haloalkyl, —NHCO(C₁-C₆ alkyl), C₁-C₆ alkoxy, —CON(C₁-C₆ alkyl)₂, or —S(O)₀₋₂NH₂, or R³¹ and R³³, together with the atoms to which they are attached, form a 6-membered aryl optionally substituted with one or more R³⁴ independently selected from halogen, —NO₂, and —CN.

In certain such embodiments, X is —OH. In certain such embodiments, R²³ and R²⁴ are each independently hydrogen or halogen (e.g., —F, —Cl, or —Br). In certain such embodiments, R²³ is hydrogen and R²⁴ is halogen (e.g., —F, —Cl, or —Br) or C₁-C₆ alkoxy (e.g., —OCH₃). In certain such embodiments, R²³ and R²⁴ are each hydrogen. In certain such embodiments, R²⁵ and R²⁶ are each independently hydrogen, halogen (e.g., —F, —Cl, or —Br), C₁-C₆ alkyl (e.g., —CH₃), or —OH. In certain such embodiments, R²⁵ is aryl optionally substituted with one or more (e.g., two) R³⁴ selected from halogen (e.g., —F), —NO₂, and —CN. In certain such embodiments, R²⁶ is halogen (e.g., —Cl) or —OH. In certain such embodiments, R³¹ is halogen (e.g., —F or —Cl), R³² is —NO₂, —CN, halogen (e.g., —F), or C₁-C₆ haloalkyl (e.g., CF₃), and R³³ is hydrogen.

In certain desirable embodiments as otherwise described herein, X is —NHR²²; R²² and R²³, together with the atoms to which they are attached, form a 5-membered heteroaryl; R²⁴ is hydrogen, halogen (e.g., —F, —Cl, or —Br), or C₁-C₆ alkoxy (e.g., —OCH₃); R²⁵ and R²⁶ are each independently hydrogen, halogen (e.g., —F, —Cl, or —Br), —NO₂, C₁-C₆ alkyl (e.g., —CH₃), —OH, or —CO(C₁-C₆ alkyl) (e.g., —CO(CH₃)); and

### Y is

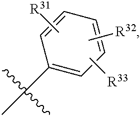

wherein R³¹, R³², and R³³ are each independently hydrogen, halogen, —NO₂, —CN, C₁-C₆ alkyl, C₁-C₆ haloalkyl, —NHCO(C₁-C₆ alkyl), C₁-C₆ alkoxy, —CON(C₁-C₆ alkyl)₂, or —S(O)₀₋₂NH₂, or R³¹ and R³³, together with the atoms to which they are attached, form a 6-membered aryl optionally substituted with one or more R³⁴ independently selected from halogen, —NO₂, and —CN.

In certain such embodiments, the compound is

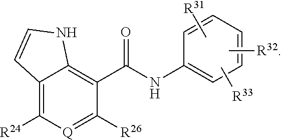

In certain such embodiments, R²⁴ is hydrogen. In certain such embodiments, Q is CR²⁵, and R²⁵ and R²⁶ are each independently hydrogen, halogen (e.g., —F, —Cl, or —Br), C₁-C₆ alkyl (e.g., —CH₃), or —OH. In certain such embodiments, R²⁵ and R²⁶ are each hydrogen. In certain such embodiments, R³¹ is halogen (e.g., —F or —Cl), R³² is —NO₂, —CN, halogen (e.g., —F, —Cl, or —Br), or C₁-C₆ haloalkyl (e.g., CF₃), and R³³ is hydrogen.

In certain desirable embodiments as otherwise described herein, X is —OR²¹; R²¹ is hydrogen, C₁-C₆ alkyl (e.g., C₁-C₄ alkyl), amino(C₁-C₆ alkyl) (e.g., amino(C₁-C₄ alkyl)), or heterocycloalkyl (e.g., piperidinyl); R²³ and R²⁶ are hydrogen, R²⁴ is hydrogen, halogen (e.g., —F, —Cl, or —Br), or C₁-C₆ alkoxy (e.g., —OCH₃), and Q is CR²⁵, and R²⁵ is hydrogen, halogen (e.g., —F, —Cl, or —Br), —NO₂, C₁-C₆ alkyl (e.g., —CH₃), —CO(C₁-C₆ alkyl) (e.g., —CO(CH₃)), or aryl optionally substituted with one or more (e.g., two) R³⁴ independently selected from halogen (e.g., —F), —NO₂, and —CN, and at least one or R²⁴ and R²⁵ is not hydrogen; and

### Y is

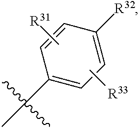

wherein R³¹ is hydrogen or halogen (e.g., —F, —Cl, or —Br), R³² is hydrogen, halogen (e.g., —F, —Cl, or —Br), —NO₂, —CN, C₁-C₆ haloalkyl (e.g., CF₃), or —S(O)₀₋₂NH₂, R³³ is hydrogen, halogen (e.g., —F, —Cl, or —Br), —NO₂, or C₁-C₆ alkoxy (e.g., —OCH₃), or R³¹ and R³³, together with the atoms to which they are attached, form a 6-membered aryl, and at least one of R³¹, R³², and R³³ is not hydrogen.

In certain such embodiments, X is —OH. In certain such embodiments, R²⁴ is halogen (e.g., —Cl) or C₁-C₆ alkoxy (e.g., —OCH₃), and Q is CR²⁵, and R²⁵ is hydrogen. In certain such embodiments, R²⁴ is hydrogen, and Q is CR²⁵, and R²⁵ is halogen (e.g., —F, —Cl, or —Br), —NO₂, C₁-C₆ alkyl (e.g., —CH₃), —CO(C₁-C₆ alkyl) (e.g., —CO(CH₃)), or aryl optionally substituted with one or more (e.g., two) R³⁴ independently selected from halogen (e.g., —F), —NO₂, and —CN. In certain such embodiments, R³¹ is halogen (e.g., —F or —Cl), R³² is —NO₂, —CN, halogen (e.g., —F), or C₁-C₆ haloalkyl (e.g., CF₃), and R³³ is hydrogen. In certain such embodiments, R³¹ and R³² are each independently hydrogen or halogen (e.g., —F, —Cl, or —Br), and R¹³ is —NO₂.

Particularly useful compounds of Formula II are provided in Table 2.

TABLE 2

| No. | Structure | Compound |
|---|---|---|
| 2 | 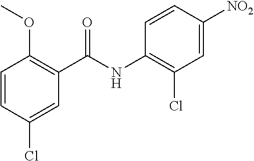 [SMILES: COc1ccc(Cl)cc1C(=O)Nc1ccc([N+](=O)[O-])cc1Cl] | 5-chloro-N-(2-chloro-4-nitrophenyl)-2- methoxybenzamide |
| 3 | 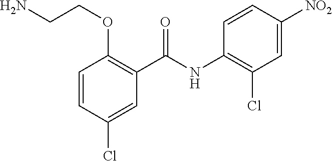 [SMILES: NCCOc1ccc(Cl)cc1C(=O)Nc1ccc([N+](=O)[O-])cc1Cl] | 2-(2-aminoethoxy)-5-chloro-N-(2-chloro- 4-nitrophenyl)benzamide |
| 4 | 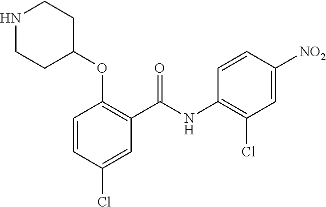 [SMILES: O=C(Nc1ccc([N+](=O)[O-])cc1Cl)c1cc(Cl)ccc1OC1CCNCC1] | 5-chloro-N-(2-chloro-4-nitrophenyl)-2- (piperidin-4-yloxy)benzamide |
| 5 | 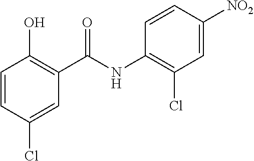 [SMILES: O=C(Nc1ccc([N+](=O)[O-])cc1Cl)c1cc(Cl)ccc1O] | N-(2-chloro-4-nitrophenyl)-5-fluoro-2- hydroxybenzamide |
| 6 | 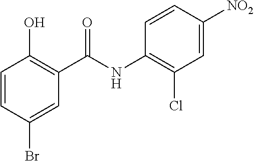 [SMILES: O=C(Nc1ccc([N+](=O)[O-])cc1Cl)c1cc(Br)ccc1O] | 5-bromo-N-(2-chloro-4-nitrophenyl)-2- hydroxybenzamide |
| 7 | 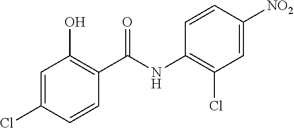 [SMILES: O=C(Nc1ccc([N+](=O)[O-])cc1Cl)c1ccc(Cl)cc1O] | 4-chloro-N-(2-chloro-4-nitrophenyl)-2- hydroxybenzamide |
| 8 | 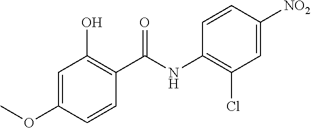 [SMILES: COc1ccc(C(=O)Nc2ccc([N+](=O)[O-])cc2Cl)c(O)c1] | N-(2-chloro-4-nitrophenyl)-2-hydroxy-4- methoxybenzamide |
| 9 | 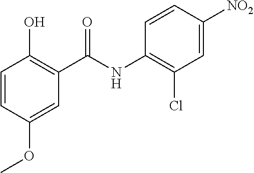 [SMILES: COc1ccc(O)c(C(=O)Nc2ccc([N+](=O)[O-])cc2Cl)c1] | N-(2-chloro-4-nitrophenyl)-2-hydroxy-5- methoxybenzamide |
| 10 | 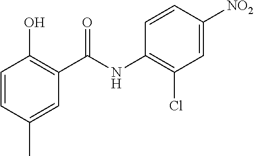 [SMILES: Cc1ccc(O)c(C(=O)Nc2ccc([N+](=O)[O-])cc2Cl)c1] | N-(2-chloro-4-nitrophenyl)-2-hydroxy-5- methylbenzamide |
| 11 | 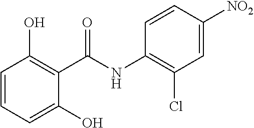 [SMILES: O=C(Nc1ccc([N+](=O)[O-])cc1Cl)c1c(O)cccc1O] | N-(2-chloro-4-nitrophenyl)-2,6- dihydroxybenzamide |
| 12 | 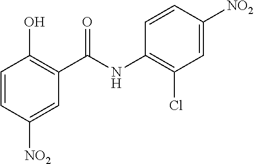 [SMILES: O=C(Nc1ccc([N+](=O)[O-])cc1Cl)c1cc([N+](=O)[O-])ccc1O] | N-(2-chloro-4-nitrophenyl)-2-hydroxy-5- nitrobenzamide |
| 13 | 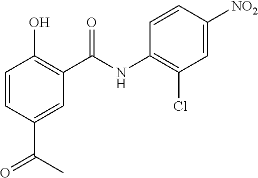 [SMILES: CC(=O)c1ccc(O)c(C(=O)Nc2ccc([N+](=O)[O-])cc2Cl)c1] | 5-acetyl-N-(2-chloro-4-nitrophenyl)-2- hydroxybenzamide |
| 14 | 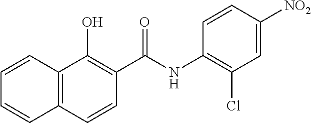 [SMILES: O=C(Nc1ccc([N+](=O)[O-])cc1Cl)c1ccc2ccccc2c1O] | N-(2-chloro-4-nitrophenyl)-1-hydroxy-2- naphthamide |
| 15 | 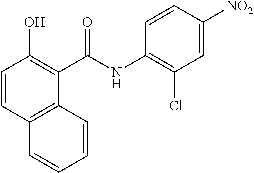 [SMILES: O=C(Nc1ccc([N+](=O)[O-])cc1Cl)c1c(O)ccc2ccccc12] | N-(2-chloro-4-nitrophenyl)-2-hydroxy-1- naphthamide |
| 16 | 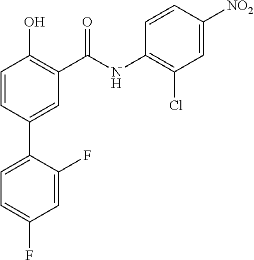 [SMILES: O=C(Nc1ccc([N+](=O)[O-])cc1Cl)c1cc(-c2ccc(F)cc2F)ccc1O] | N-(2-chloro-4-nitrophenyl)-2′,4′-difluoro-4- hydroxy-[1,1′-biphenyl]-3-carboxamide |
| 17 | 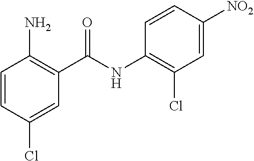 [SMILES: Nc1ccc(Cl)cc1C(=O)Nc1ccc([N+](=O)[O-])cc1Cl] | 2-amino-5-chloro-N-(2-chloro-4- nitrophenyl)benzamide |
| 18 | 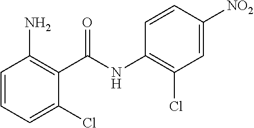 [SMILES: Nc1cccc(Cl)c1C(=O)Nc1ccc([N+](=O)[O-])cc1Cl] | 2-amino-6-chloro-N-(2-chloro-4- nitrophenyl)benzamide |
| 19 | 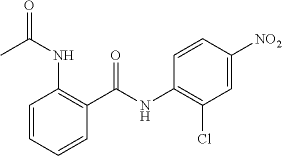 [SMILES: CC(=O)Nc1ccccc1C(=O)Nc1ccc([N+](=O)[O-])cc1Cl] | 2-acetamido-N-(2-chloro-4- nitrophenyl)benzamide |
| 20 | 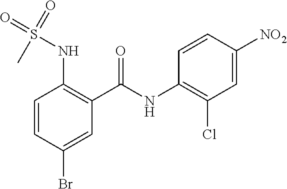 [SMILES: CS(=O)(=O)Nc1ccc(Br)cc1C(=O)Nc1ccc([N+](=O)[O-])cc1Cl] | 5-bromo-N-(2-chloro-4-nitrophenyl)-2- (methylsulfonamido)benzamide |
| 21 | 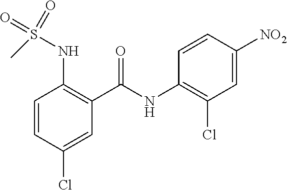 [SMILES: CS(=O)(=O)Nc1ccc(Cl)cc1C(=O)Nc1ccc([N+](=O)[O-])cc1Cl] | 5-chloro-N-(2-chloro-4-nitrophenyl)-2- (phenylsulfonamido)benzamide |
| 22 | 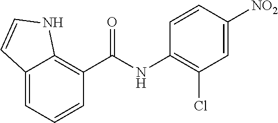 [SMILES: O=C(Nc1ccc([N+](=O)[O-])cc1Cl)c1cccc2cc[nH]c12] | N-(2-chloro-4-nitrophenyl)-1H-indole-7- carboxamide |
| 23 | 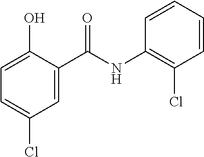 [SMILES: O=C(Nc1ccccc1Cl)c1cc(Cl)ccc1O] | 5-chloro-N-(2-chlorophenyl)-2- hydroxybenzamide |
| 24 | 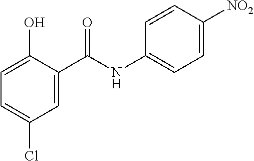 [SMILES: O=C(Nc1ccc([N+](=O)[O-])cc1)c1cc(Cl)ccc1O] | 5-chloro-2-hydroxy-N-(4- nitrophenyl)benzamide |
| 25 | 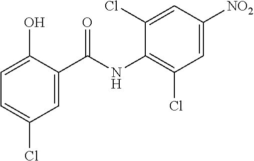 [SMILES: O=C(Nc1c(Cl)cc([N+](=O)[O-])cc1Cl)c1cc(Cl)ccc1O] | 5-chloro-N-(2,6-dichloro-4-nitrophenyl)-2- hydroxybenzamide |
| 26 | 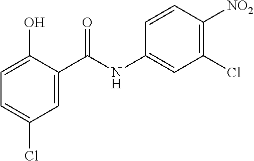 [SMILES: O=C(Nc1ccc([N+](=O)[O-])c(Cl)c1)c1cc(Cl)ccc1O] | 5-chloro-N-(3-chloro-4-nitrophenyl)-2- hydroxybenzamide |
| 27 | 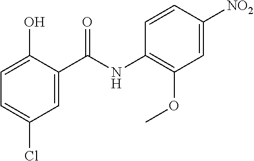 [SMILES: COc1cc([N+](=O)[O-])ccc1NC(=O)c1cc(Cl)ccc1O] | 5-chloro-2-hydroxy-N-(2-methoxy-4- nitrophenyl)benzamide |
| 28 | 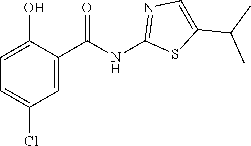 [SMILES: CC(C)c1cnc(NC(=O)c2cc(Cl)ccc2O)s1] | 5-chloro-2-hydroxy-N-(5-isopropylthiazol- 2-yl)benzamide |
| 29 | 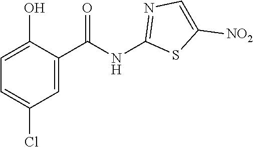 [SMILES: O=C(Nc1ncc([N+](=O)[O-])s1)c1cc(Cl)ccc1O] | 5-chloro-2-hydroxy-N-(5-nitrothiazol-2- yl)benzamide |
| 30 | 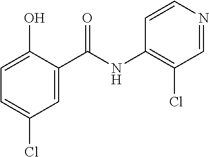 [SMILES: O=C(Nc1ccncc1Cl)c1cc(Cl)ccc1O] | 5-chloro-N-(3-chloropyridin-4-yl)-2- hydroxybenzamide |
| 31 | 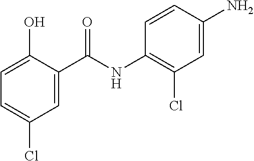 [SMILES: Nc1ccc(NC(=O)c2cc(Cl)ccc2O)c(Cl)c1] | N-(4-amino-2-chlorophenyl)-5-chloro-2- hydroxybenzamide |
| 32 | 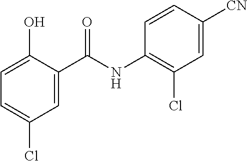 [SMILES: N#Cc1ccc(NC(=O)c2cc(Cl)ccc2O)c(Cl)c1] | 5-chloro-N-(2-chloro-4-cyanophenyl)-2- hydroxybenzamide |
| 33 | 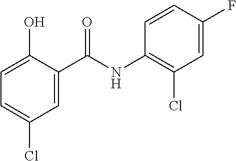 [SMILES: O=C(Nc1ccc(F)cc1Cl)c1cc(Cl)ccc1O] | 5-chloro-N-(2-chloro-4-fluorophenyl)-2- hydroxybenzamide |
| 34 | 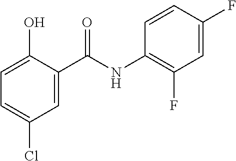 [SMILES: O=C(Nc1ccc(F)cc1F)c1cc(Cl)ccc1O] | 5-chloro-N-(2,4-difluorophenyl)-2- hydroxybenzamide |
| 35 | 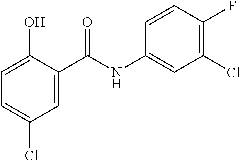 [SMILES: O=C(Nc1ccc(F)c(Cl)c1)c1cc(Cl)ccc1O] | 5-chloro-N-(3-chloro-4-fluorophenyl)-2- hydroxybenzamide |
| 36 | 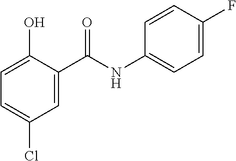 [SMILES: O=C(Nc1ccc(F)cc1)c1cc(Cl)ccc1O] | 5-chloro-N-(4-fluorophenyl)-2- hydroxybenzamide |
| 37 | 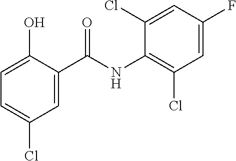 [SMILES: O=C(Nc1c(Cl)cc(F)cc1Cl)c1cc(Cl)ccc1O] | 5-chloro-N-(2,6-dichloro-4-fluorophenyl)- 2-hydroxybenzamide |
| 38 | 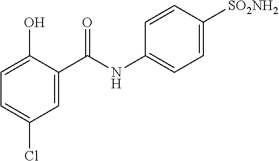 [SMILES: NS(=O)(=O)c1ccc(NC(=O)c2cc(Cl)ccc2O)cc1] | 5-chloro-2-hydroxy-N-(4- sulfamoylphenyl)benzamide |
| 39 | 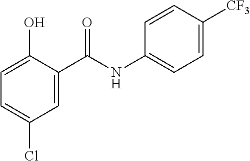 [SMILES: O=C(Nc1ccc(C(F)(F)F)cc1)c1cc(Cl)ccc1O] | 5-chloro-2-hydroxy-N-(4- (trifluoromethyl)phenyl)benzamide |
| 40 | 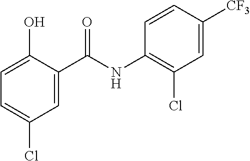 [SMILES: O=C(Nc1ccc(C(F)(F)F)cc1Cl)c1cc(Cl)ccc1O] | 5-chloro-N-(2-chloro-4- (trifluoromethyl)phenyl)-2- hydroxybenzamide |
| 41 | 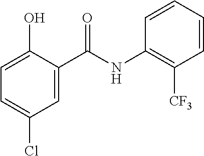 [SMILES: O=C(Nc1ccccc1C(F)(F)F)c1cc(Cl)ccc1O] | 5-chloro-2-hydroxy-N-(2- (trifluoromethyl)phenyl)benzamide |
| 42 | 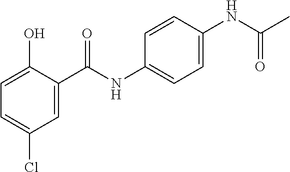 [SMILES: CC(=O)Nc1ccc(NC(=O)c2cc(Cl)ccc2O)cc1] | N-(4-acetamidophenyl)-5-chloro-2- hydroxybenzamide |
| 43 | 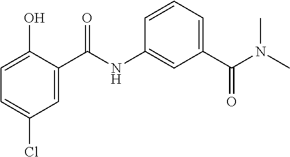 [SMILES: CN(C)C(=O)c1cccc(NC(=O)c2cc(Cl)ccc2O)c1] | 5-chloro-N-(3- (dimethylcarbamoyl)phenyl)-2- hydroxybenzamide |
| 44 | 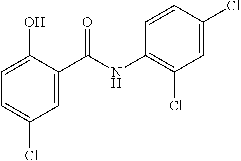 [SMILES: O=C(Nc1ccc(Cl)cc1Cl)c1cc(Cl)ccc1O] | 5-chloro-N-(2,4-dichlorophenyl)-2- hydroxybenzamide |
| 45 | 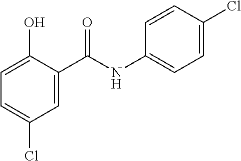 [SMILES: O=C(Nc1ccc(Cl)cc1)c1cc(Cl)ccc1O] | 5-chloro-N-(4-chlorophenyl)-2- hydroxybenzamide |
| 46 | 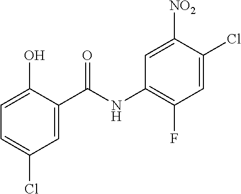 [SMILES: O=C(Nc1cc([N+](=O)[O-])c(Cl)cc1F)c1cc(Cl)ccc1O] | 5-chloro-N-(4-chloro-2-fluoro-5- nitrophenyl)-2-hydroxybenzamide |
| 47 | 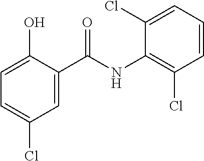 [SMILES: O=C(Nc1c(Cl)cccc1Cl)c1cc(Cl)ccc1O] | 5-chloro-N-(2,6-dichlorophenyl)-2- hydroxybenzamide |
| 48 | 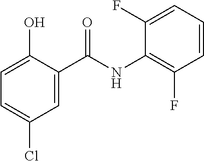 [SMILES: O=C(Nc1c(F)cccc1F)c1cc(Cl)ccc1O] | 5-chloro-N-(2,6-difluorophenyl)-2- hydroxybenzamide |
| 49 | 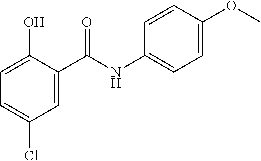 [SMILES: COc1ccc(NC(=O)c2cc(Cl)ccc2O)cc1] | 5-chloro-2-hydroxy-N-(4- methoxyphenyl)benzamide |
| 50 | 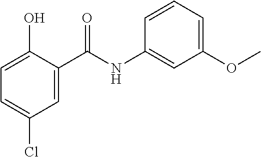 [SMILES: COc1cccc(NC(=O)c2cc(Cl)ccc2O)c1] | 5-chloro-2-hydroxy-N-(3- methoxyphenyl)benzamide |
| 51 | 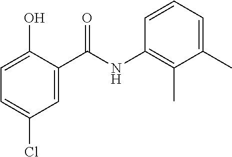 [SMILES: Cc1cccc(NC(=O)c2cc(Cl)ccc2O)c1C] | 5-chloro-N-(2,3-dimethylphenyl)-2- hydroxybenzamide |
| 52 | 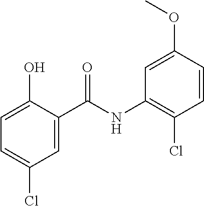 [SMILES: COc1ccc(Cl)c(NC(=O)c2cc(Cl)ccc2O)c1] | 5-chloro-N-(2-chloro-5-methoxyphenyl)-2- hydroxybenzamide |
| 53 | 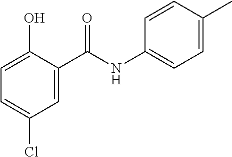 [SMILES: Cc1ccc(NC(=O)c2cc(Cl)ccc2O)cc1] | 5-chloro-2-hydroxy-N-(p-tolyl)benzamide |
| 54 | 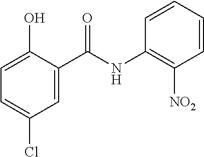 [SMILES: O=C(Nc1ccccc1[N+](=O)[O-])c1cc(Cl)ccc1O] | 5-chloro-2-hydroxy-N-(2- nitrophenyl)benzamide |
| 55 | 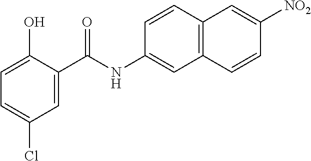 [SMILES: O=C(Nc1ccc2cc([N+](=O)[O-])ccc2c1)c1cc(Cl)ccc1O] | 5-chloro-2-hydroxy-N-(6-nitronaphthalen- 2-yl)benzamide |
| 56 | 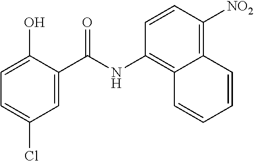 [SMILES: O=C(Nc1ccc([N+](=O)[O-])c2ccccc12)c1cc(Cl)ccc1O] | 5-chloro-2-hydroxy-N-(4-nitronaphthalen- 1-yl)benzamide |
| 57 | 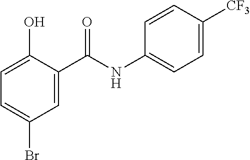 [SMILES: O=C(Nc1ccc(C(F)(F)F)cc1)c1cc(Br)ccc1O] | 5-bromo-2-hydroxy-N-(4- (trifluoromethyl)phenyl)benzamide |
| 58 | 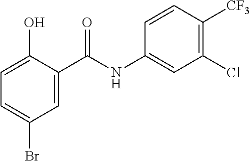 [SMILES: O=C(Nc1ccc(C(F)(F)F)c(Cl)c1)c1cc(Br)ccc1O] | 5-bromo-N-(3-chloro-4- (trifluoromethyl)phenyl)-2- hydroxybenzamide |
| 59 | 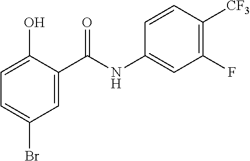 [SMILES: O=C(Nc1ccc(C(F)(F)F)c(F)c1)c1cc(Br)ccc1O] | 5-bromo-N-(3-fluoro-4- (trifluoromethyl)phenyl)-2- hydroxybenzamide |
| 60 | 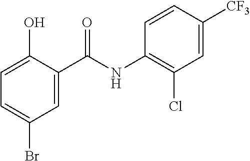 [SMILES: O=C(Nc1ccc(C(F)(F)F)cc1Cl)c1cc(Br)ccc1O] | 5-bromo-N-(2-chloro-4- (trifluoromethyl)phenyl)-2- hydroxybenzamide |
| 61 | 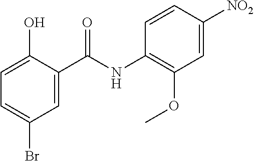 [SMILES: COc1cc([N+](=O)[O-])ccc1NC(=O)c1cc(Br)ccc1O] | 5-bromo-2-hydroxy-N-(2-methoxy-4- nitrophenyl)benzamide |
| 62 | 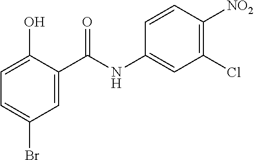 [SMILES: O=C(Nc1ccc([N+](=O)[O-])c(Cl)c1)c1cc(Br)ccc1O] | 5-bromo-N-(3-chloro-4-nitrophenyl)-2- hydroxybenzamide |
| 63 | 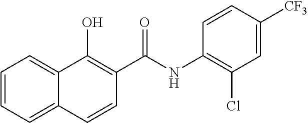 [SMILES: O=C(Nc1ccc(C(F)(F)F)cc1Cl)c1ccc2ccccc2c1O] | N-(2-chloro-4-(trifluoromethyl)phenyl)-1- hydroxy-2-naphthamide |
| 64 | 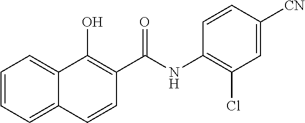 [SMILES: N#Cc1ccc(NC(=O)c2ccc3ccccc3c2O)c(Cl)c1] | N-(2-chloro-4-cyanophenyl)-1-hydroxy-2- naphthamide |
| 65 | 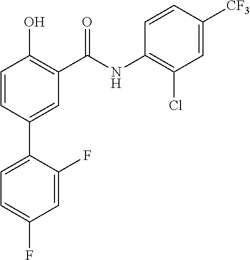 [SMILES: O=C(Nc1ccc(C(F)(F)F)cc1Cl)c1cc(-c2ccc(F)cc2F)ccc1O] | N-(2-chloro-4-(trifluoromethyl)phenyl)- 2′,4′-difluoro-4-hydroxy-[1,1′-biphenyl]-3- carboxamide |
| 66 | 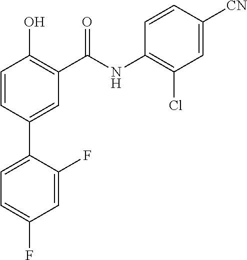 [SMILES: N#Cc1ccc(NC(=O)c2cc(-c3ccc(F)cc3F)ccc2O)c(Cl)c1] | N-(2-chloro-4-cyanophenyl)-2′,4′-difluoro- 4-hydroxy-[1,1′-biphenyl]-3-carboxamide |
| 67 | 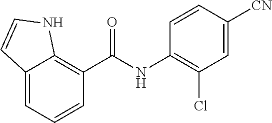 [SMILES: N#Cc1ccc(NC(=O)c2cccc3cc[nH]c23)c(Cl)c1] | N-(2-chloro-4-cyanophenyl)-1H-indole-7- carboxamide |
| 68 | 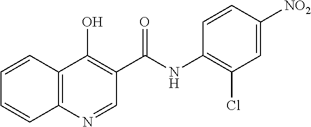 [SMILES: O=C(Nc1ccc([N+](=O)[O-])cc1Cl)c1cnc2ccccc2c1O] | N-(2-chloro-4-nitrophenyl)-4- hydroxyquinoline-3-carboxamide |

In certain embodiments, the compound of Formula II is selected from those listed in Table 2.

In certain embodiments, the compound of Formula II is compound 6, 22, 24, 27, 32, 39, or 59.

### Pharmaceutical Compositions

In another aspect, the present disclosure provides pharmaceutical compositions comprising one or more of compounds as described herein, and a pharmaceutically acceptable carrier, excipient, adjuvant, and/or diluent. The exact nature of the carrier, excipient, adjuvant, and/or diluent will depend upon the desired use for the composition.

In certain embodiments, the pharmaceutical composition comprises one or more compounds of Formula I or a pharmaceutically acceptable salt thereof (i.e., as otherwise described herein), and a pharmaceutically acceptable carrier, excipient, adjuvant, and/or diluent. In certain embodiments, the pharmaceutical composition comprises one or more compounds of Formula II or a pharmaceutically acceptable salt thereof (i.e., as otherwise described herein), and a pharmaceutically acceptable carrier, excipient, adjuvant, and/or diluent.

Pharmaceutical compositions comprising the compound(s) may be manufactured by means of conventional mixing, dissolving, granulating, dragee-making levigating, emulsifying, encapsulating, entrapping or lyophilization processes. The compositions may be formulated in conventional manner using one or more physiologically acceptable carriers, diluents, excipients or auxiliaries which facilitate processing of the compounds into preparations which can be used pharmaceutically.

The compounds may be formulated in the pharmaceutical composition per se, or in the form of a hydrate, solvate, N-oxide or pharmaceutically acceptable salt, as previously described. Typically, such salts are more soluble in aqueous solutions than the corresponding free acids and bases, but salts having lower solubility than the corresponding free acids and bases may also be formed.

Pharmaceutical compositions may take a form suitable for virtually any mode of administration, including, for example, topical, ocular, oral, buccal, systemic, nasal, injection, transdermal, rectal, vaginal, etc., or a form suitable for administration by inhalation or insufflation.

For topical administration, the compound(s) may be formulated as solutions, gels, ointments, creams, suspensions, etc. as are well-known in the art. Systemic formulations include those designed for administration by injection, e.g., subcutaneous, intravenous, intramuscular, intrathecal or intraperitoneal injection, as well as those designed for transdermal, transmucosal oral or pulmonary administration.

Useful injectable preparations include sterile suspensions, solutions or emulsions of the active compound(s) in aqueous or oily vehicles. The compositions may also contain formulating agents, such as suspending, stabilizing and/or dispersing agent. The formulations for injection may be presented in unit dosage form, e.g., in ampules or in multidose containers, and may contain added preservatives. Alternatively, the injectable formulation may be provided in powder form for reconstitution with a suitable vehicle, including but not limited to sterile pyrogen free water, buffer, dextrose solution, etc., before use. To this end, the active compound(s) may be dried by any art-known technique, such as lyophilization, and reconstituted prior to use.

For transmucosal administration, penetrants appropriate to the barrier to be permeated are used in the formulation. Such penetrants are known in the art.

For oral administration, the pharmaceutical compositions may take the form of, for example, lozenges, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulfate). The tablets may be coated by methods well known in the art with, for example, sugars, films or enteric coatings.

Liquid preparations for oral administration may take the form of, for example, elixirs, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol, Cremophore™ or fractionated vegetable oils); and preservatives (e.g., methyl or propyl-p-hydroxybenzoates or sorbic acid). The preparations may also contain buffer salts, preservatives, flavoring, coloring and sweetening agents as appropriate.

Preparations for oral administration may be suitably formulated to give controlled release of the compound, as is well known. For buccal administration, the compositions may take the form of tablets or lozenges formulated in conventional manner. For rectal and vaginal routes of administration, the compound(s) may be formulated as solutions (for retention enemas) suppositories or ointments containing conventional suppository bases such as cocoa butter or other glycerides.

For nasal administration or administration by inhalation or insufflation, the compound(s) can be conveniently delivered in the form of an aerosol spray from pressurized packs or a nebulizer with the use of a suitable propellant, e.g., dichlorodifluoromethane, trichloro-fluoromethane, dichlorotetrafluoroethane, fluorocarbons, carbon dioxide or other suitable gas. In the case of a pressurized aerosol, the dosage unit may be determined by providing a valve to deliver a metered amount. Capsules and cartridges for use in an inhaler or insufflator (for example capsules and cartridges comprised of gelatin) may be formulated containing a powder mix of the compound and a suitable powder base such as lactose or starch.

For ocular administration, the compound(s) may be formulated as a solution, emulsion, suspension, etc. suitable for administration to the eye. A variety of vehicles suitable for administering compounds to the eye are known in the art.

For prolonged delivery, the compound(s) can be formulated as a depot preparation for administration by implantation or intramuscular injection. The compound(s) may be formulated with suitable polymeric or hydrophobic materials (e.g., as an emulsion in an acceptable oil) or ion exchange resins, or as sparingly soluble derivatives, e.g., as a sparingly soluble salt. Alternatively, transdermal delivery systems manufactured as an adhesive disc or patch which slowly releases the compound(s) for percutaneous absorption may be used. To this end, permeation enhancers may be used to facilitate transdermal penetration of the compound(s).

Alternatively, other pharmaceutical delivery systems may be employed. Liposomes and emulsions are well-known examples of delivery vehicles that may be used to deliver compound(s). Certain organic solvents such as dimethyl sulfoxide (DMSO) may also be employed, although usually at the cost of greater toxicity.

The pharmaceutical compositions may, if desired, be presented in a pack or dispenser device which may contain one or more unit dosage forms containing the compound(s). The pack may, for example, comprise metal or plastic foil, such as a blister pack. The pack or dispenser device may be accompanied by instructions for administration.

The compound(s) described herein, or compositions thereof, will generally be used in an amount effective to achieve the intended result, for example in an amount effective to treat or prevent the particular disease being treated. By therapeutic benefit is meant eradication or amelioration of the underlying disorder being treated and/or eradication or amelioration of one or more of the symptoms associated with the underlying disorder such that the patient reports an improvement in feeling or condition, notwithstanding that the patient may still be afflicted with the underlying disorder. Therapeutic benefit also generally includes halting or slowing the progression of the disease, regardless of whether improvement is realized.

The amount of compound(s) administered will depend upon a variety of factors, including, for example, the particular indication being treated, the mode of administration, whether the desired benefit is prophylactic or therapeutic, the severity of the indication being treated and the age and weight of the patient, the bioavailability of the particular compound(s) the conversation rate and efficiency into active drug compound under the selected route of administration, etc.

Determination of an effective dosage of compound(s) for a particular use and mode of administration is well within the capabilities of those skilled in the art. Effective dosages may be estimated initially from in vitro activity and metabolism assays. For example, an initial dosage of compound for use in animals may be formulated to achieve a circulating blood or serum concentration of the metabolite active compound that is at or above an IC₅₀ of the particular compound as measured in as in vitro assay. Calculating dosages to achieve such circulating blood or serum concentrations taking into account the bioavailability of the particular compound via the desired route of administration is well within the capabilities of skilled artisans. Initial dosages of compound can also be estimated from in vivo data, such as animal models. Animal models useful for testing the efficacy of the active metabolites to treat or prevent the various diseases described above are well-known in the art. Animal models suitable for testing the bioavailability and/or metabolism of compounds into active metabolites are also well-known. Ordinarily skilled artisans can routinely adapt such information to determine dosages of particular compounds suitable for human administration.

Dosage amounts will typically be in the range of from about 0.0001 mg/kg/day, 0.001 mg/kg/day or 0.01 mg/kg/day to about 100 mg/kg/day, but may be higher or lower, depending upon, among other factors, the activity of the active compound, the bioavailability of the compound, its metabolism kinetics and other pharmacokinetic properties, the mode of administration and various other factors, discussed above. Dosage amount and interval may be adjusted individually to provide plasma levels of the compound(s) and/or active metabolite compound(s) which are sufficient to maintain therapeutic or prophylactic effect. For example, the compounds may be administered once per week, several times per week (e.g., every other day), once per day or multiple times per day, depending upon, among other things, the mode of administration, the specific indication being treated and the judgment of the prescribing physician. In cases of local administration or selective uptake, such as local topical administration, the effective local concentration of compound(s) and/or active metabolite compound(s) may not be related to plasma concentration. Skilled artisans will be able to optimize effective dosages without undue experimentation.

In some embodiments, the pharmaceutical composition is formulated for oral administration once a day or QD, and in some such formulations is a unit where the effective amount of the active ingredient ranges from 50 mg to 5000 mg. Alternatively, an oral solution may be provided ranging from a concentration of 1 mg/ml to 50 mg/ml or higher.

One embodiment of the disclosure includes administering a compound of the disclosure to provide a serum concentration ranging from 0.1 μM to 50 μM. One embodiment of the disclosure includes administering a compound of the disclosure to provide a serum concentration ranging from 1 μM to 20 μM. One embodiment of the disclosure includes administering a compound of the disclosure to provide a serum concentration ranging from 5 μM to 20 μM. One embodiment of the disclosure includes administering a compound of the disclosure to provide a serum concentration of 10 μM, 20 μM, 5 μM, 1 μM, 15 μM, or 40 μM.

One embodiment of the disclosure includes administering a compound of the disclosure at a dose of 1 to 100 mg/kg/day, 5-40 mg/kg/day, 10-20 mg/kg/day, 1-2 mg/kg/day, 20-40 mg/kg/day, 45-50 mg/kg/day, 50-60 mg/kg/day, 55-65 mg/kg/day, 60-70 mg/kg/day or 65-75 mg/kg/day.

The compositions described herein may be given in one dose, but is not restricted to one dose. Thus, the administration can be two, three, four, five, six, seven, eight, nine, ten, eleven, twelve, thirteen, fourteen, fifteen, sixteen, seventeen, eighteen, nineteen, twenty, or more, administrations of the vaccine. Where there is more than one administration in the present methods, the administrations can be spaced by time intervals of one minute, two minutes, three, four, five, six, seven, eight, nine, ten, or more minutes, by intervals of about one hour, two hours, three, four, five, six, seven, eight, nine, ten, 11, 12, 13, 14, 15, 16, 17, 18, 19, 20, 21, 22, 23, 24 hours, and so on. In the context of hours, the term “about” means plus or minus any time interval within 30 minutes. The administrations can also be spaced by time intervals of one day, two days, three days, four days, five days, six days, seven days, eight days, nine days, ten days, 11 days, 12 days, 13 days, 14 days, 15 days, 16 days, 17 days, 18 days, 19 days, 20 days, 21 days, and combinations thereof. The disclosure is not limited to dosing intervals that are spaced equally in time, but encompass doses at non-equal intervals, such as a priming schedule consisting of administration at 1 day, 4 days, 7 days, and 25 days, just to provide a non-limiting example.

A dosing schedule of, for example, once/week, twice/week, three times/week, four times/week, five times/week, six times/week, seven times/week, once every two weeks, once every three weeks, once every four weeks, once every five weeks, and the like, is available for the invention. The dosing schedules encompass dosing for a total period of time of, for example, one week, two weeks, three weeks, four weeks, five weeks, six weeks, two months, three months, four months, five months, six months, seven months, eight months, nine months, ten months, eleven months, and twelve months.

Provided are cycles of the above dosing schedules. The cycle can be repeated about, e.g., every seven days; every 14 days; every 21 days; every 28 days; every 35 days; 42 days; every 49 days; every 56 days; every 63 days; every 70 days; and the like. An interval of non-dosing can occur between a cycle, where the interval can be about, e.g., seven days; 14 days; 21 days; 28 days; 35 days; 42 days; 49 days; 56 days; 63 days; 70 days; and the like. In this context, the term “about” means plus or minus one day, plus or minus two days, plus or minus three days, plus or minus four days, plus or minus five days, plus or minus six days, or plus or minus seven days.

As one aspect of the present disclosure contemplates the treatment of the disease/conditions with the compounds of the disclosure, the disclosure further relates to pharmaceutical compositions in kit form. When the composition of the disclosure is a part of a combination therapy with a secondary therapeutic agent, the kit may comprise two separate pharmaceutical compositions: one of compound of the present disclosure, and another of a second therapeutic agent. The kit comprises a container for containing the separate compositions such as a divided bottle or a divided foil packet. Additional examples of containers include syringes, boxes, and bags. In some embodiments, the kit comprises directions for the use of the separate components. The kit form is particularly advantageous when the separate components are preferably administered in different dosage forms (e.g., oral and parenteral), are administered at different dosage intervals, or when titration of the individual components of the combination is desired by the prescribing health care professional.

The compounds and compositions of the disclosure as described herein may also be administered in combination with one or more secondary therapeutic agents. Thus, in certain embodiments, the method also includes administering to a subject in need of such treatment an effective amount of one or more compounds of the disclosure as described herein (e.g., compounds of Formula I or Formula II, or those provided in Tables 1 and 2) or a pharmaceutical composition of the disclosure as described herein and one or more secondary therapeutic agents.

Combination therapy, in defining use of a compound of the present disclosure and the secondary therapeutic agent, is intended to embrace administration of each agent in a sequential manner in a regimen that will provide beneficial effects of the drug combination (e.g., the compounds and compositions of the disclosure as described herein and the secondary therapeutic agents can be formulated as separate compositions that are given sequentially), and is intended as well to embrace co-administration of these agents in a substantially simultaneous manner, such as in a single pharmaceutical composition having a fixed ratio of these active agents or in multiple or a separate pharmaceutical compositions for each agent. The disclosure is not limited in the sequence of administration: the compounds of and compositions of the disclosure may be administered either prior to or after (i.e., sequentially), or at the same time (i.e., simultaneously) as administration of the secondary therapeutic agent.

In certain embodiments, the secondary therapeutic agent may be administered in a previously established clinical dose when dosed for therapy in humans. In certain embodiments, the secondary therapeutic agent may be administered in an amount below its established human clinical dose when dosed for therapy. For example, the secondary therapeutic agent may be administered in an amount less than 1% of, e.g., less than 10%, or less than 25%, or less than 50%, or less than 75%, or even less than 90% of the established human clinical dose.

Examples of secondary therapeutic agents include, but not limited to, steroids (such as, but are not limited to, dexamethasone, cortisone, hydrocortisone, hydrocortisone acetate, cortisone acetate, prednisolone, methylprednisolone, prednisone, betamethasone, betamethasone dipropionate, betamethasone valerate, clobetasol propionate, clobetasone, fluprednidene acetate, hydrocortisone aceponate, hydrocortisone buteprate, hydrocortisone butyrate, hydrocortisone valerate, fluocortolone, halometasone, mometasone, and prednicarbate), nonsteroidal anti-inflammatory drugs (NSAIDs) (such as, but not limited to, indomethacin, sulindac, ibuprofen, aspirin, naproxen, and tolmetin), immunomodulating agents (such as, but not limited to, azathioprine, cyclosporine, cyclophosphamide, deoxyspergualin, bredinin, rituximab, tocilizumab, sirolimus, methotrexate, anti CD3 antibodies, anti CD19 antibody, anti CD22 antibody, folinic acid, cyclosphosphamide, mycophenolate mofetil, and a B-cell targeting agent), chemotherapy agents (such as, but not limited to, didemnin B, dehydrodidemnin B, and bortezomib), intravenous gamma globulin (IVIG), thalidomide, inebilizumab, vascular health agents (such as, but not limited to, anticoagulants, antiplatelet agents, angiotensin-converting enzyme inhibitors, angiotensin II receptor blockers, angiotensin-receptor neprilysin inhibitors, beta blockers, calcium channel blockers, cholesterol-lowering medications, diuretics, and vasodilators), and convalescent plasma. Other examples of secondary therapeutic agents include, but are not limited to, viral-targeting antivirals (such as, but not limited to, polymerase inhibitors such as remdesivir, EIDD-2801, and other nucleoside analogs such as ribavirin, favipiravir, gemcitabine, mizoribine, and mycophenolate; protease inhibitors such as HIV-1 protease inhibitors such as nelfinavir, atazanavir, 3CLpro investigational inhibitors and PLpro investigational inhibitors; and entry inhibitors such as monoclonal antibodies, fusion inhibitors, soluble ACE2 and molecular decoys, heparan sulfate proteoglycan, lactoferrin, spike mimicking peptides, and griffithsin), interferons, and host-targeting compounds (such as, but not limited to, protease inhibitors targeting TMPRSS2 and furin (camostat, nafamostat (TMPRSS2 mediated) and MI-1851 (furin mediated)); apilimod; DHODH inhibitors such as leflunomide; chloroquine and its analogs; chloropromazine; signaling pathway inhibitors such as kinase inhibitors including dasatinib and imatinib; interferon inducers such as ampligen; cyclophilin inhibitors such as cyclosporin and FK506; and ivermectin); and other compounds such as ciclesonide, nitazoxanide, and emetine.

### Definitions

Throughout this specification, unless the context requires otherwise, the word “comprise” and “include” and variations (e.g., “comprises,” “comprising,” “includes,” “including”) will be understood to imply the inclusion of a stated component, feature, element, or step or group of components, features, elements or steps but not the exclusion of any other integer or step or group of integers or steps.

As used in the specification and the appended claims, the singular forms “a,” “an” and “the” include plural referents unless the context clearly dictates otherwise.

Terms used herein may be preceded and/or followed by a single dash, “-”, or a double dash, “=”, to indicate the bond order of the bond between the named substituent and its parent moiety; a single dash indicates a single bond and a double dash indicates a double bond. In the absence of a single or double dash it is understood that a single bond is formed between the substituent and its parent moiety; further, substituents are intended to be read “left to right” (i.e., the attachment is via the last portion of the name) unless a dash indicates otherwise. For example, C₁-C₆alkoxycarbonyloxy and —OC(O)C₁-C₆alkyl indicate the same functionality; similarly arylalkyl and -alkylaryl indicate the same functionality.

The term “alkenyl” as used herein, means a straight or branched chain hydrocarbon containing from 2 to 10 carbons, unless otherwise specified, and containing at least one carbon-carbon double bond. Representative examples of alkenyl include, but are not limited to, ethenyl, 2-propenyl, 2-methyl-2-propenyl, 3-butenyl, 4-pentenyl, 5-hexenyl, 2-heptenyl, 2-methyl-1-heptenyl, 3-decenyl, and 3,7-dimethylocta-2,6-dienyl.

The term “alkoxy” as used herein, means an alkyl group, as defined herein, appended to the parent molecular moiety through an oxygen atom. Representative examples of alkoxy include, but are not limited to, methoxy, ethoxy, propoxy, 2-propoxy, butoxy, tert-butoxy, pentyloxy, and hexyloxy.

The term “alkyl” as used herein, means a straight or branched chain hydrocarbon containing from 1 to 10 carbon atoms unless otherwise specified. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl. When an “alkyl” group is a linking group between two other moieties, then it may also be a straight or branched chain; examples include, but are not limited to —CH₂—, —CH₂CH₂—, —CH₂CH₂CHC(CH₃)—, and —CH₂CH(CH₂CH₃)CH₂—.

The term “alkylene” refers to a bivalent alkyl group. An “alkylene chain” is a polymethylene group, i.e., —(CH₂)ₙ—, wherein n is a positive integer, preferably from one to six, from one to four, from one to three, from one to two, or from two to three. A substituted alkylene chain is a polymethylene group in which one or more methylene hydrogen atoms is replaced with a substituent. Suitable substituents include those described below for a substituted aliphatic group. An alkylene chain also may be substituted at one or more positions with an aliphatic group or a substituted aliphatic group.

The term “alkynyl” as used herein, means a straight or branched chain hydrocarbon group containing from 2 to 10 carbon atoms and containing at least one carbon-carbon triple bond. Representative examples of alkynyl include, but are not limited, to acetylenyl, 1-propynyl, 2-propynyl, 3-butynyl, 2-pentynyl, and 1-butynyl.

The term “aryl,” as used herein, means a phenyl (i.e., monocyclic aryl), or a bicyclic ring system containing at least one phenyl ring or an aromatic bicyclic ring containing only carbon atoms in the aromatic bicyclic ring system. The bicyclic aryl can be azulenyl, naphthyl, or a phenyl fused to a monocyclic cycloalkyl, a monocyclic cycloalkenyl, or a monocyclic heterocyclyl. The bicyclic aryl is attached to the parent molecular moiety through any carbon atom contained within the phenyl portion of the bicyclic system, or any carbon atom with the napthyl or azulenyl ring. The fused monocyclic cycloalkyl or monocyclic heterocyclyl portions of the bicyclic aryl are optionally substituted with one or two oxo and/or thia groups. Representative examples of the bicyclic aryls include, but are not limited to, azulenyl, naphthyl, dihydroinden-1-yl, dihydroinden-2-yl, dihydroinden-3-yl, dihydroinden-4-yl, 2,3-dihydroindol-4-yl, 2,3-dihydroindol-5-yl, 2,3-dihydroindol-6-yl, 2,3-dihydroindol-7-yl, inden-1-yl, inden-2-yl, inden-3-yl, inden-4-yl, dihydronaphthalen-2-yl, dihydronaphthalen-3-yl, dihydronaphthalen-4-yl, dihydronaphthalen-1-yl, 5,6,7,8-tetrahydronaphthalen-1-yl, 5,6,7,8-tetrahydronaphthalen-2-yl, 2,3-dihydrobenzofuran-4-yl, 2,3-dihydrobenzofuran-5-yl, 2,3-dihydrobenzofuran-6-yl, 2,3-dihydrobenzofuran-7-yl, benzo[d][1,3]dioxol-4-yl, benzo[d][1,3]dioxol-5-yl, 2H-chromen-2-on-5-yl, 2H-chromen-2-on-6-yl, 2H-chromen-2-on-7-yl, 2H-chromen-2-on-8-yl, isoindoline-1,3-dion-4-yl, isoindoline-1,3-dion-5-yl, inden-1-on-4-yl, inden-1-on-5-yl, inden-1-on-6-yl, inden-1-on-7-yl, 2,3-dihydrobenzo[b][1,4]dioxan-5-yl, 2,3-dihydrobenzo[b][1,4]dioxan-6-yl, 2H-benzo[b][1,4]oxazin3(4H)-on-5-yl, 2H-benzo[b][1,4]oxazin3(4H)-on-6-yl, 2H-benzo[b][1,4]oxazin3(4H)-on-7-yl, 2H-benzo[b][1,4]oxazin3(4H)-on-8-yl, benzo[d]oxazin-2(3H)-on-5-yl, benzo[d]oxazin-2(3H)-on-6-yl, benzo[d]oxazin-2(3H)-on-7-yl, benzo[d]oxazin-2(3H)-on-8-yl, quinazolin-4(3H)-on-5-yl, quinazolin-4(3H)-on-6-yl, quinazolin-4(3H)-on-7-yl, quinazolin-4(3H)-on-8-yl, quinoxalin-2(1H)-on-5-yl, quinoxalin-2(1H)-on-6-yl, quinoxalin-2(1H)-on-7-yl, quinoxalin-2(1H)-on-8-yl, benzo[d]thiazol-2(3H)-on-4-yl, benzo[d]thiazol-2(3H)-on-5-yl, benzo[d]thiazol-2(3H)-on-6-yl, and, benzo[d]thiazol-2(3H)-on-7-yl. In certain embodiments, the bicyclic aryl is (i) naphthyl or (ii) a phenyl ring fused to either a 5 or 6 membered monocyclic cycloalkyl, a 5 or 6 membered monocyclic cycloalkenyl, or a 5 or 6 membered monocyclic heterocyclyl, wherein the fused cycloalkyl, cycloalkenyl, and heterocyclyl groups are optionally substituted with one or two groups which are independently oxo or thia.

The terms “cyano” and “nitrile” as used herein, mean a —CN group.

The term “cycloalkyl” as used herein, means a monocyclic or a bicyclic cycloalkyl ring system. Monocyclic ring systems are cyclic hydrocarbon groups containing from 3 to 8 carbon atoms, where such groups can be saturated or unsaturated, but not aromatic. In certain embodiments, cycloalkyl groups are fully saturated. Examples of monocyclic cycloalkyls include cyclopropyl, cyclobutyl, cyclopentyl, cyclopentenyl, cyclohexyl, cyclohexenyl, cycloheptyl, and cyclooctyl. Bicyclic cycloalkyl ring systems are bridged monocyclic rings or fused bicyclic rings. Bridged monocyclic rings contain a monocyclic cycloalkyl ring where two non-adjacent carbon atoms of the monocyclic ring are linked by an alkylene bridge of between one and three additional carbon atoms (i.e., a bridging group of the form —(CH₂)_{w}—, where w is 1, 2, or 3). Representative examples of bicyclic ring systems include, but are not limited to, bicyclo[3.1.1]heptane, bicyclo[2.2.1]heptane, bicyclo[2.2.2]octane, bicyclo[3.2.2]nonane, bicyclo[3.3.1]nonane, and bicyclo[4.2.1]nonane. Fused bicyclic cycloalkyl ring systems contain a monocyclic cycloalkyl ring fused to either a phenyl, a monocyclic cycloalkyl, a monocyclic cycloalkenyl, a monocyclic heterocyclyl, or a monocyclic heteroaryl. The bridged or fused bicyclic cycloalkyl is attached to the parent molecular moiety through any carbon atom contained within the monocyclic cycloalkyl ring. Cycloalkyl groups are optionally substituted with one or two groups which are independently oxo or thia. In certain embodiments, the fused bicyclic cycloalkyl is a 5 or 6 membered monocyclic cycloalkyl ring fused to either a phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the fused bicyclic cycloalkyl is optionally substituted by one or two groups which are independently oxo or thia.

The term “halo” or “halogen” as used herein, means —Cl, —Br, —I or —F.

The terms “haloalkyl” and “haloalkoxy” refer to an alkyl or alkoxy group, as the case may be, which is substituted with one or more halogen atoms.

The term “heteroaryl,” as used herein, means a monocyclic heteroaryl or a bicyclic ring system containing at least one heteroaromatic ring. The monocyclic heteroaryl can be a 5 or 6 membered ring. The 5 membered ring consists of two double bonds and one, two, three or four nitrogen atoms and optionally one oxygen or sulfur atom. The 6 membered ring consists of three double bonds and one, two, three or four nitrogen atoms. The 5 or 6 membered heteroaryl is connected to the parent molecular moiety through any carbon atom or any nitrogen atom contained within the heteroaryl. Representative examples of monocyclic heteroaryl include, but are not limited to, furyl, imidazolyl, isoxazolyl, isothiazolyl, oxadiazolyl, oxazolyl, pyridinyl, pyridazinyl, pyrimidinyl, pyrazinyl, pyrazolyl, pyrrolyl, tetrazolyl, thiadiazolyl, thiazolyl, thienyl, triazolyl, and triazinyl. The bicyclic heteroaryl consists of a monocyclic heteroaryl fused to a phenyl, a monocyclic cycloalkyl, a monocyclic cycloalkenyl, a monocyclic heterocyclyl, or a monocyclic heteroaryl. The fused cycloalkyl or heterocyclyl portion of the bicyclic heteroaryl group is optionally substituted with one or two groups which are independently oxo or thia. When the bicyclic heteroaryl contains a fused cycloalkyl, cycloalkenyl, or heterocyclyl ring, then the bicyclic heteroaryl group is connected to the parent molecular moiety through any carbon or nitrogen atom contained within the monocyclic heteroaryl portion of the bicyclic ring system. When the bicyclic heteroaryl is a monocyclic heteroaryl fused to a benzo ring, then the bicyclic heteroaryl group is connected to the parent molecular moiety through any carbon atom or nitrogen atom within the bicyclic ring system. Representative examples of bicyclic heteroaryl include, but are not limited to, benzimidazolyl, benzofuranyl, benzothienyl, benzoxadiazolyl, benzoxathiadiazolyl, benzothiazolyl, cinnolinyl, 5,6-dihydroquinolin-2-yl, 5,6-dihydroisoquinolin-1-yl, furopyridinyl, indazolyl, indolyl, isoquinolinyl, naphthyridinyl, quinolinyl, purinyl, 5,6,7,8-tetrahydroquinolin-2-yl, 5,6,7,8-tetrahydroquinolin-3-yl, 5,6,7,8-tetrahydroquinolin-4-yl, 5,6,7,8-tetrahydroisoquinolin-1-yl, thienopyridinyl, 4,5,6,7-tetrahydrobenzo[c][1,2,5]oxadiazolyl, 2,3-dihydrothieno[3,4-b][1,4]dioxan-5-yl, and 6,7-dihydrobenzo[c][1,2,5]oxadiazol-4(5H)-onyl. In certain embodiments, the fused bicyclic heteroaryl is a 5 or 6 membered monocyclic heteroaryl ring fused to either a phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the fused cycloalkyl, cycloalkenyl, and heterocyclyl groups are optionally substituted with one or two groups which are independently oxo or thia.

The terms “heterocyclyl” and “heterocycloalkyl” as used herein, mean a monocyclic heterocycle or a bicyclic heterocycle. The monocyclic heterocycle is a 3, 4, 5, 6, 7, or 8 membered ring containing at least one heteroatom independently selected from the group consisting of O, N, and S where the ring is saturated or unsaturated, but not aromatic. The 3 or 4 membered ring contains 1 heteroatom selected from the group consisting of O, N and S. The 5 membered ring can contain zero or one double bond and one, two or three heteroatoms selected from the group consisting of O, N and S. The 6 or 7 membered ring contains zero, one or two double bonds and one, two or three heteroatoms selected from the group consisting of O, N and S. The monocyclic heterocycle is connected to the parent molecular moiety through any carbon atom or any nitrogen atom contained within the monocyclic heterocycle. Representative examples of monocyclic heterocycle include, but are not limited to, azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, oxadiazolinyl, oxadiazolidinyl, oxazolinyl, oxazolidinyl, piperazinyl, piperidinyl, pyranyl, pyrazolinyl, pyrazolidinyl, pyrrolinyl, pyrrolidinyl, tetrahydrofuranyl, tetrahydrothienyl, thiadiazolinyl, thiadiazolidinyl, thiazolinyl, thiazolidinyl, thiomorpholinyl, 1,1-dioxidothiomorpholinyl (thiomorpholine sulfone), thiopyranyl, and trithianyl. The bicyclic heterocycle is a bridged monocyclic ring or a monocyclic heterocycle fused to either a phenyl, a monocyclic cycloalkyl, a monocyclic cycloalkenyl, a monocyclic heterocycle, or a monocyclic heteroaryl. Bridged monocyclic rings contain a monocyclic heterocycloalkyl ring where two non-adjacent carbon atoms of the monocyclic ring are linked by an alkylene bridge of between one and three additional carbon atoms (i.e., a bridging group of the form —(CH₂)_{w}—, where w is 1, 2, or 3). The bicyclic heterocycle is connected to the parent molecular moiety through any carbon atom or any nitrogen atom contained within the monocyclic heterocycle portion of the bicyclic ring system. Representative examples of bicyclic heterocyclyls include, but are not limited to, 2,3-dihydrobenzofuran-2-yl, 2,3-dihydrobenzofuran-3-yl, indolin-1-yl, indolin-2-yl, indolin-3-yl, 2,3-dihydrobenzothien-2-yl, decahydroquinolinyl, decahydroisoquinolinyl, octahydro-1H-indolyl, and octahydrobenzofuranyl. Heterocyclyl groups are optionally substituted with one or two groups which are independently oxo or thia. In certain embodiments, the bicyclic heterocyclyl is a 5 or 6 membered monocyclic heterocyclyl ring fused to phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the bicyclic heterocyclyl is optionally substituted by one or two groups which are independently oxo or thia.

The term “oxo” as used herein means a ═O group.

The term “saturated” as used herein means the referenced chemical structure does not contain any multiple carbon-carbon bonds. For example, a saturated cycloalkyl group as defined herein includes cyclohexyl, cyclopropyl, and the like.

The term “substituted”, as used herein, means that a hydrogen radical of the designated moiety is replaced with the radical of a specified substituent, provided that the substitution results in a stable or chemically feasible compound. The term “substitutable”, when used in reference to a designated atom, means that attached to the atom is a hydrogen radical, which can be replaced with the radical of a suitable substituent.

The phrase “one or more” substituents, as used herein, refers to a number of substituents that equals from one to the maximum number of substituents possible based on the number of available bonding sites, provided that the above conditions of stability and chemical feasibility are met. Unless otherwise indicated, an optionally substituted group may have a substituent at each substitutable position of the group, and the substituents may be either the same or different. As used herein, the term “independently selected” means that the same or different values may be selected for multiple instances of a given variable in a single compound.

The term “thia” as used herein means a ═S group.

The term “unsaturated” as used herein means the referenced chemical structure contains at least one multiple carbon-carbon bond, but is not aromatic. For example, an unsaturated cycloalkyl group as defined herein includes cyclohexenyl, cyclopentenyl, cyclohexadienyl, and the like.

It will be apparent to one skilled in the art that certain compounds of this disclosure may exist in tautomeric forms, all such tautomeric forms of the compounds being within the scope of the disclosure. Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure. Both the R and the S stereochemical isomers, as well as all mixtures thereof, are included within the scope of the disclosure.

“Pharmaceutically acceptable” refers to those compounds, materials, compositions, and/or dosage forms which are, within the scope of sound medical judgment, suitable for contact with the tissues of human beings and animals without excessive toxicity, irritation, allergic response, or other problems or complications commensurate with a reasonable benefit/risk ratio or which have otherwise been approved by the United States Food and Drug Administration as being acceptable for use in humans or domestic animals.

“Pharmaceutically acceptable salt” refers to both acid and base addition salts.

“Therapeutically effective amount” or “effective amount” refers to that amount of a compound which, when administered to a subject, is sufficient to effect treatment for a disease or disorder described herein. The amount of a compound which constitutes a “therapeutically effective amount” will vary depending on the compound, the disorder and its severity, and the age of the subject to be treated, but can be determined routinely by one of ordinary skill in the art. An effective amount is one that will decrease or ameliorate the symptoms normally by at least 10%, more normally by at least 20%, most normally by at least 30%, typically by at least 40%, more typically by at least 50%, most typically by at least 60%, often by at least 70%, more often by at least 80%, and most often by at least 90%, conventionally by at least 95%, more conventionally by at least 99%, and most conventionally by at least 99.9%.

“Treating” or “treatment” as used herein covers the treatment of a disease or disorder described herein, in a subject, preferably a human, and includes:
i. inhibiting a disease or disorder, i.e., arresting its development;
ii. relieving a disease or disorder, i.e., causing regression of the disorder;
iii. slowing progression of the disorder; and/or
iv. inhibiting, relieving, ameliorating, or slowing progression of one or more symptoms of the disease or disorder.

“Subject” refers to a warm blooded animal such as a mammal, preferably a human, or a human child, which is afflicted with, or has the potential to be afflicted a disease as described herein.

### Methods of Preparation

Many general references providing commonly known chemical synthetic schemes and conditions useful for synthesizing the disclosed compounds are available (see, e.g., Smith and March, March's Advanced Organic Chemistry: Reactions, Mechanisms, and Structure, Fifth Edition, Wiley-Interscience, 2001; or Vogel, A Textbook of Practical Organic Chemistry, Including Qualitative Organic Analysis, Fourth Edition, New York: Longman, 1978).

Compounds as described herein can be purified by any of the means known in the art, including chromatographic means, such as HPLC, preparative thin layer chromatography, flash column chromatography and ion exchange chromatography. Any suitable stationary phase can be used, including normal and reversed phases as well as ionic resins. Most typically the disclosed compounds are purified via silica gel and/or alumina chromatography. See, e.g., Introduction to Modern Liquid Chromatography, 2nd Edition, ed. L. R. Snyder and J. J. Kirkland, John Wiley and Sons, 1979; and Thin Layer Chromatography, ed E. Stahl, Springer-Verlag, New York, 1969.

During any of the processes for preparation of the subject compounds, it may be necessary and/or desirable to protect sensitive or reactive groups on any of the molecules concerned. This may be achieved by means of conventional protecting groups as described in standard works, such as J. F. W. McOmie, “Protective Groups in Organic Chemistry,” Plenum Press, London and New York 1973, in T. W. Greene and P. G. M. Wuts, “Protective Groups in Organic Synthesis,” Third edition, Wiley, New York 1999, in “The Peptides”; Volume 3 (editors: E. Gross and J. Meienhofer), Academic Press, London and New York 1981, in “Methoden der organischen Chemie,” Houben-Weyl, 4.sup.th edition, Vol. 15/1, Georg Thieme Verlag, Stuttgart 1974, in H.-D. Jakubke and H. Jescheit, “Aminosauren, Peptide, Proteine,” Verlag Chemie, Weinheim, Deerfield Beach, and Basel 1982, and/or in Jochen Lehmann, “Chemie der Kohlenhydrate: Monosaccharide and Derivate,” Georg Thieme Verlag, Stuttgart 1974. The protecting groups may be removed at a convenient subsequent stage using methods known from the art.

The compounds disclosed herein can be made using procedures familiar to the person of ordinary skill in the art and as described herein. For example, compounds of structural formula (I) can be prepared according to Schemes 1-6, general procedures (below), and/or analogous synthetic procedures. One of skill in the art can adapt the reaction sequences of Schemes 1-6, general procedures, and Examples 1-13 to fit the desired target molecule. Of course, in certain situations one of skill in the art will use different reagents to affect one or more of the individual steps or to use protected versions of certain of the substituents. Additionally, one skilled in the art would recognize that compounds of the disclosure can be synthesized using different routes altogether.

### General Procedures

Representative synthetic procedures for the preparation of compounds of the invention are outlined below in Schemes 1 and 2.

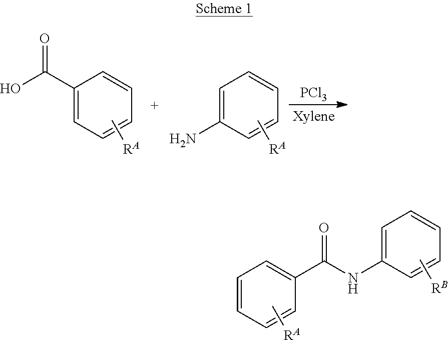

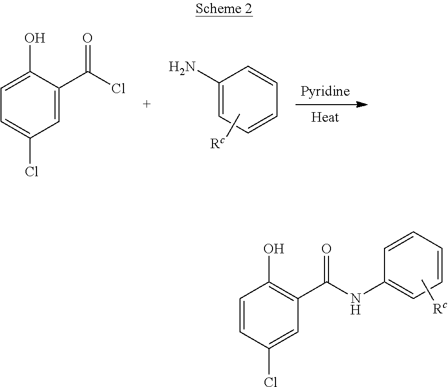

### EXAMPLES

The compositions and methods of the disclosure are illustrated further by the following examples, which are not to be construed as limiting the disclosure in scope or spirit to the specific procedures and compounds described in them.

### Example 1

### General Methods

All air or moisture sensitive reactions were performed under positive pressure of nitrogen with oven-dried glassware. Chemical reagents and anhydrous solvents were obtained from commercial sources and used as is. Preparative purification was performed on a Waters semipreparative HPLC instrument. The column used was a Phenomenex Luna C18 (5 μm, 30 mm×75 mm) at a flow rate of 45 mL/min. The mobile phase consisted of acetonitrile and water (each containing 0.1% trifluoroacetic acid). A gradient from 10% to 50% acetonitrile over 8 min was used during the purification. Fraction collection was triggered by UV detection (220 nm). Alternately, flash chromatography on silica gel was performed using forced flow (liquid) of the indicated solvent system on Biotage KP-Sil pre-packed cartridges and using the Biotage SP-1 automated chromatography system.

Analytical analysis for purity was determined by two different methods denoted as final QC methods 1 and 2:

Method 1. Analysis was performed on an Agilent 1290 Infinity series HPLC instrument. UHPLC long gradient equivalent from 4% to 100% acetonitrile (0.05% trifluoroacetic acid) in water over 3 min run time of 4.5 min with a flow rate of 0.8 mL/min. A Phenomenex Luna C18 column (3 μm, 3 mm×75 mm) was used at a temperature of 50° C.

Method 2. Analysis was performed on an Agilent 1260 with a 7 min gradient from 4% to 100% acetonitrile (containing 0.025% trifluoroacetic acid) in water (containing 0.05% trifluoroacetic acid) over 8 min run time at a flow rate of 1 mL/min. A Phenomenex Luna C18 column (3 μm, 3 mm×75 mm) was used at a temperature of 50° C.

Purity determination was performed using an Agilent diode array detector for both method 1 and method 2. Mass determination was performed using an Agilent 6130 mass spectrometer with electrospray ionization in the positive mode. All the analogs for assay have purity greater than 95% based on both analytical methods. ¹H NMR spectra were recorded on Varian 400 MHz spectrometers. High resolution mass spectrometry results were recorded on Agilent 6210 time-of-flight LC/MS system.

### Compounds 2-68: Representative Procedures

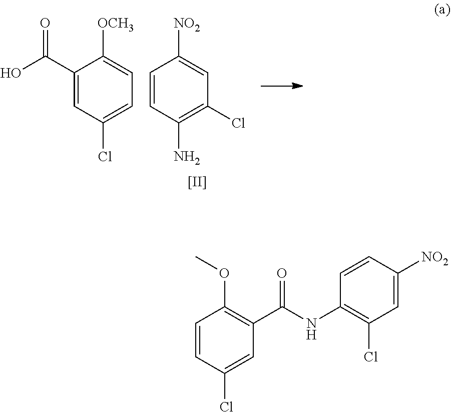

Compound 2: 5-chloro-2-methoxybenzoic acid (0.1 g, 0.536 mmol) and 2-chloro-4-nitroaniline (0.092 g, 0.536 mmol) were suspended in dry Xylene (2.68 mL). After heating to reflux, Phosphorus trichloride (0.019 mL, 0.214 mmol) was added and the reaction mixture stirred under reflux for four hours. The reaction mixture was then cooled to RT and an equal volume of water was added. The mixture was stirred for 30 min and then pH was adjusted to >8 by adding sat. NaHCO₃. The resultant mixture was then filtered and washed with Hexanes to get the dry solid. DMSO (5 mL) was added and the compound was dissolved via heating. On cooling, the pure compound crashed out. It was then filtered, washed with hexanes and dried to provide 5-chloro-N-(2-chloro-4-nitrophenyl)-2-methoxybenzamide (0.125 g, 0.366 mmol, 68.4% yield) as the pure product. ¹H NMR (400 MHz, DMSO-d₆) δ 10.92 (s, 1H), 8.76 (d, J=9.2 Hz, 1H), 8.47 (d, J=2.6 Hz, 1H), 8.31 (dd, J=9.3, 2.6 Hz, 1H), 8.01 (d, J=2.7 Hz, 1H), 7.72 (dd, J=8.9, 2.8 Hz, 1H), 7.39 (d, J=8.9 Hz, 1H), 4.12 (s, 3H). LC-MS (method 2): t_{R}=6.67 min, m/z (M+H)⁺=342.

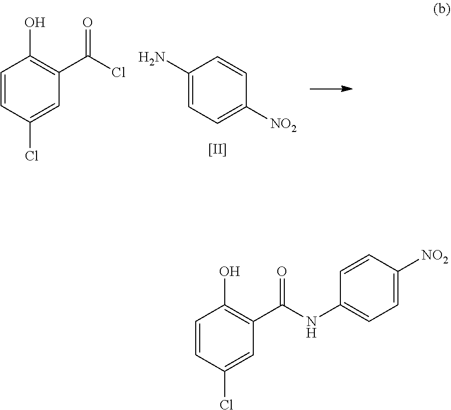

Compound 24: 5-chloro-2-hydroxybenzoyl chloride (0.166 g, 0.869 mmol) and 4-nitroaniline (0.060 g, 0.434 mmol) were suspended in Pyridine (1.5 mL) and stirred at room temperature for 1 h. After this time it was heated at 90° C. for 2 h. The reaction mixture was then cooled to room temperature, diluted with EtOAc (20 mL), washed with 10% CuSO₄ solution (20 mL), washed with 1N HCl solution, separated from aqueous layer, dried and concentrated to give the crude product which was purified by reverse phase HPLC. LC-MS (method 2): t_{R}=5.66 min, m/z (M+H)⁺=293.

### Compounds 2-68

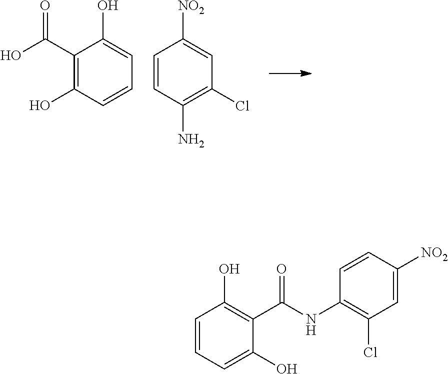

Compound 11 was synthesized according to the representative procedure (a). LC-MS (method 2): t_{R}=5.63 min, m/z (M+H)⁺=309. ¹H NMR (400 MHz, DMSO-d₆) δ 12.65 (s, 2H), 11.98 (br s, 1H), 8.71 (d, J=9.2 Hz, 1H), 8.45 (d, J=1.9 Hz, 1H), 8.30 (dd, J=9.2, 1.9 Hz, 1H), 7.31 (t, J=8.2 Hz, 1H), 6.48 (d, J=8.3 Hz, 2H).

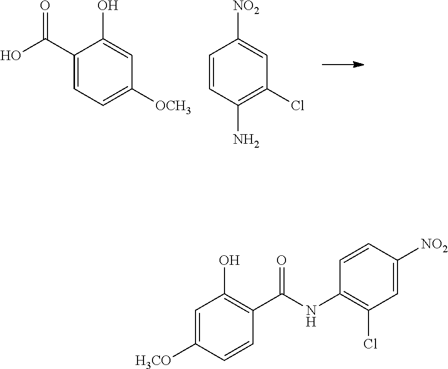 [SMILES: COc1ccc(C(=O)O)c(O)c1 ; Nc1ccc([N+](=O)[O-])cc1Cl ; COc1ccc(C(=O)Nc2ccc([N+](=O)[O-])cc2Cl)c(O)c1]

Compound 8 was synthesized according to the representative procedure (a). LC-MS (method 2): t_{R}=5.54 min, m/z (M+H)⁺=323. ¹H NMR (400 MHz, DMSO-d₆) δ 12.21 (s, 1H), 8.83 (d, J=9.2 Hz, 1H), 8.39 (d, J=2.7 Hz, 1H), 8.25 (dd, J=9.3, 2.7 Hz, 1H), 7.95 (d, J=8.9 Hz, 1H), 6.53 (d, J=25.9 Hz, 2H), 3.77 (d, J=4.3 Hz, 3H). LC-MS (method 2): t_{R}=5.54 min, m/z (M+H)⁺=323.

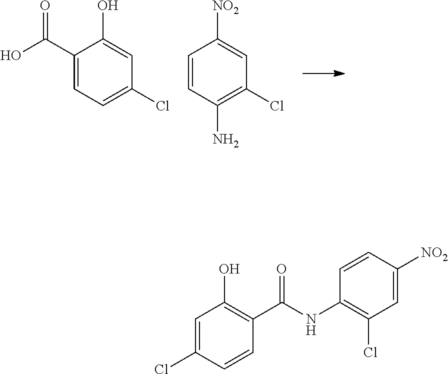

Compound 7 was synthesized according to the representative procedure (a). LC-MS (method 2): t_{R}=5.94 min, m/z (M+H)⁺=328. ¹H NMR (400 MHz, DMSO-d₆) δ 12.74 (s, 1H), 11.54 (s, 1H), 8.84 (d, J=9.3 Hz, 1H), 8.44 (d, J=2.6 Hz, 1H), 8.30 (dd, J=9.3, 2.7 Hz, 1H), 8.04 (d, J=9.1 Hz, 1H), 7.11-7.03 (m, 2H).

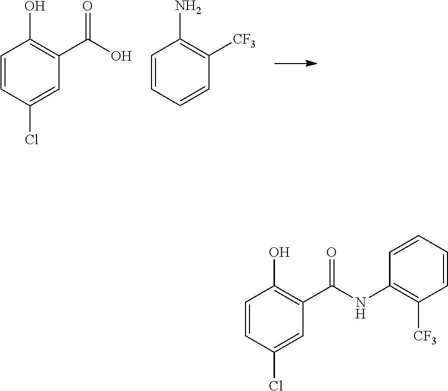

Compound 41 was synthesized according to the representative procedure (a). LC-MS (method 2): t_{R}=5.67 min, m/z (M+H)⁺=316. ¹H NMR (400 MHz, DMSO-d₆) δ 12.25 (br s, 1H), 10.95 (br s, 1H), 8.18 (d, J=8.2 Hz, 1H), 7.97 (t, J=3.0 Hz, 1H), 7.81-7.68 (m, 2H), 7.49 (ddd, J=8.4, 4.1, 2.4 Hz, 1H), 7.44-7.37 (m, 1H), 7.08-6.99 (m, 1H).

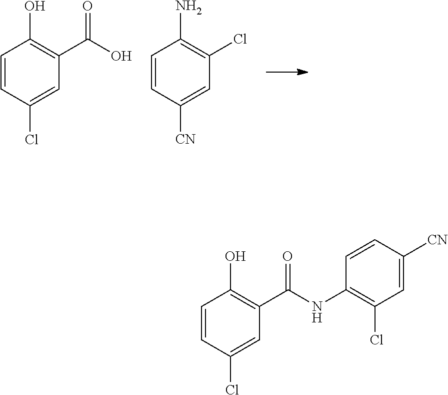

Compound 32 was synthesized according to the representative procedure (a). LC-MS (method 2): t_{R}=5.63 min, m/z (M+H)⁺=309. ¹H NMR (400 MHz, DMSO-d₆) δ 12.52 (s, 1H), 11.69 (s, 1H), 8.73 (d, J=8.7 Hz, 1H), 8.17 (d, J=1.9 Hz, 1H), 7.94 (d, J=2.8 Hz, 1H), 7.87 (dd, J=8.7, 2.0 Hz, 1H), 7.49 (dd, J=8.8, 2.8 Hz, 1H), 7.04 (d, J=8.8 Hz, 1H).

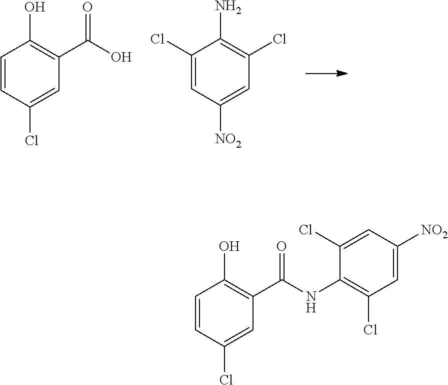 [SMILES: O=C(O)c1cc(Cl)ccc1O ; Nc1c(Cl)cc([N+](=O)[O-])cc1Cl ; O=C(Nc1c(Cl)cc([N+](=O)[O-])cc1Cl)c1cc(Cl)ccc1O]

Compound 25 was synthesized according to the representative procedure (a). LC-MS (method 2): t_{R}=5.74 min, m/z (M+H)⁺=362.

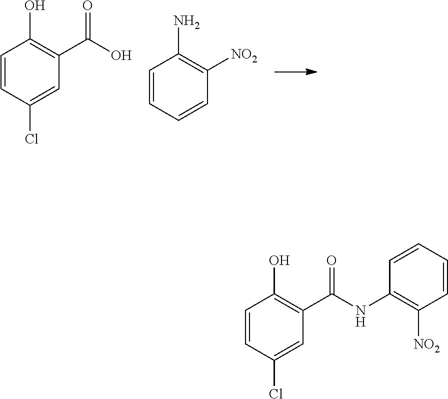

Compound 54 was synthesized according to the representative procedure (a). LC-MS (method 2): t_{R}=5.54 min, m/z (M+H)⁺=293. ¹H NMR (400 MHz, DMSO-d₆) δ 12.11 (s, 1H), 8.48 (dd, J=8.4, 1.3 Hz, 1H), 8.13 (dd, J=8.3, 1.6 Hz, 1H), 7.92 (d, J=2.8 Hz, 1H), 7.78 (ddd, J=8.4, 7.3, 1.6 Hz, 1H), 7.48 (dd, J=8.8, 2.8 Hz, 1H), 7.37 (ddd, J=8.5, 7.3, 1.3 Hz, 1H), 7.03 (d, J=8.8 Hz, 1H).

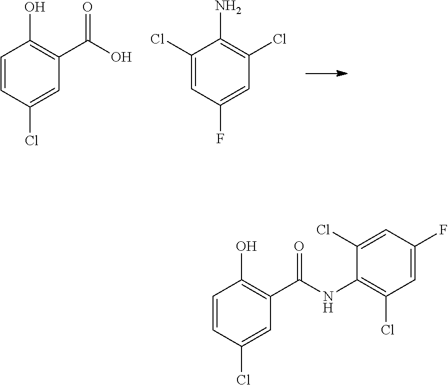 [SMILES: O=C(O)c1cc(Cl)ccc1O ; Nc1c(Cl)cc(F)cc1Cl ; O=C(Nc1c(Cl)cc(F)cc1Cl)c1cc(Cl)ccc1O]

Compound 37 was synthesized according to the representative procedure (a). LC-MS (method 2): t_{R}=5.77 min, m/z (M+H)⁺=335. ¹H NMR (400 MHz, DMSO-d₆) δ 12.11 (s, 1H), 8.48 (dd, J=8.4, 1.3 Hz, 1H), 8.13 (dd, J=8.3, 1.6 Hz, 1H), 7.92 (d, J=2.8 Hz, 1H), 7.78 (ddd, J=8.4, 7.3, 1.6 Hz, 1H), 7.48 (dd, J=8.8, 2.8 Hz, 1H), 7.37 (ddd, J=8.5, 7.3, 1.3 Hz, 1H), 7.03 (d, J=8.8 Hz, 1H).

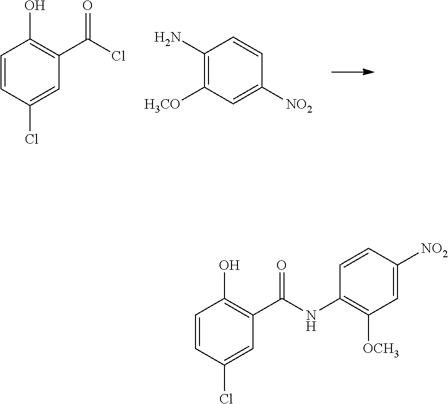 [SMILES: O=C(Cl)c1cc(Cl)ccc1O ; COc1cc([N+](=O)[O-])ccc1N ; COc1cc([N+](=O)[O-])ccc1NC(=O)c1cc(Cl)ccc1O]

Compound 27 was synthesized according to the representative procedure (b). LC-MS (method 2): t_{R}=5.74 min, m/z (M+H)⁺=323.

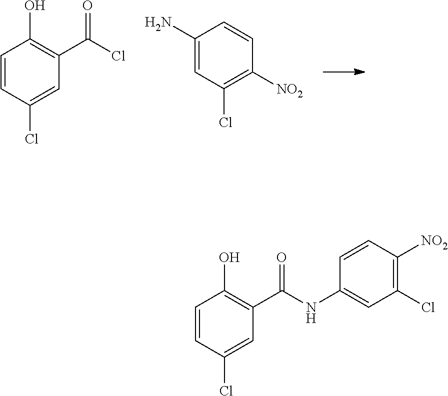 [SMILES: O=C(Cl)c1cc(Cl)ccc1O ; Nc1ccc([N+](=O)[O-])c(Cl)c1 ; O=C(Nc1ccc([N+](=O)[O-])c(Cl)c1)c1cc(Cl)ccc1O]

Compound 26 was synthesized according to the representative procedure (b). LC-MS (method 2): t_{R}=5.99 min, m/z (M+H)⁺=328.

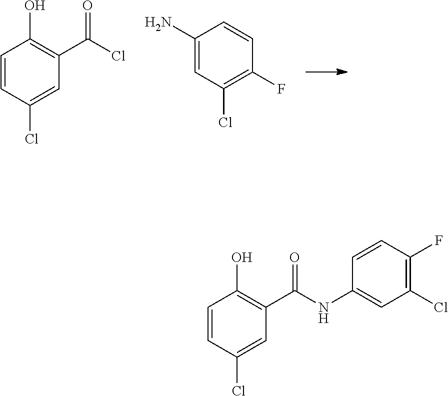 [SMILES: O=C(Cl)c1cc(Cl)ccc1O ; Nc1ccc(F)c(Cl)c1 ; O=C(Nc1ccc(F)c(Cl)c1)c1cc(Cl)ccc1O]

Compound 35 was synthesized according to the representative procedure (b). LC-MS (method 2): t_{R}=6.12 min, m/z (M+H)⁺=301. ¹H NMR (400 MHz, DMSO-d₆) δ 8.04 (dd, J=6.9, 2.6 Hz, 1H), 7.83 (d, J=2.8 Hz, 1H), 7.60 (ddd, J=9.0, 4.3, 2.6 Hz, 1H), 7.46-7.35 (m, 2H), 6.92 (d, J=8.8 Hz, 1H).

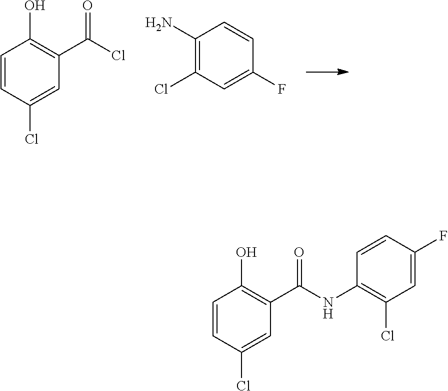 [SMILES: O=C(Cl)c1cc(Cl)ccc1O ; Nc1ccc(F)cc1Cl ; O=C(Nc1ccc(F)cc1Cl)c1cc(Cl)ccc1O]

Compound 33 was synthesized according to the representative procedure (b). LC-MS (method 2): t_{R}=5.79 min, m/z (M+H)⁺=301.

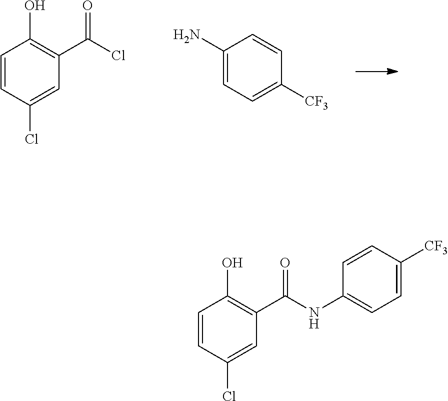 [SMILES: O=C(Cl)c1cc(Cl)ccc1O ; Nc1ccc(C(F)(F)F)cc1 ; O=C(Nc1ccc(C(F)(F)F)cc1)c1cc(Cl)ccc1O]

Compound 39 was synthesized according to the representative procedure (b). LC-MS (method 2): t_{R}=6.25 min, m/z (M+H)⁺=316. ¹H NMR (400 MHz, DMSO-d₆) δ 7.91 (d, J=8.3 Hz, 2H), 7.82 (d, J=2.8 Hz, 1H), 7.71 (d, J=8.3 Hz, 2H), 7.35 (dd, J=8.4, 2.8 Hz, 1H), 6.88 (d, J=8.8 Hz, 1H).

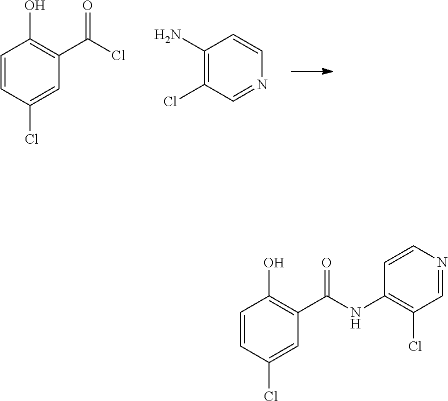 [SMILES: O=C(Cl)c1cc(Cl)ccc1O ; Nc1ccncc1Cl ; O=C(Nc1ccncc1Cl)c1cc(Cl)ccc1O]

Compound 30 was synthesized according to the representative procedure (b). LC-MS (method 2): t_{R}=4.02 min, m/z (M+H)⁺=284.

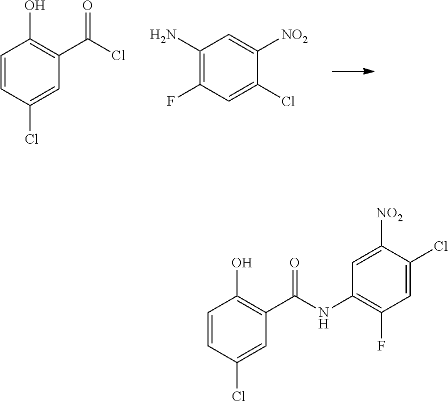 [SMILES: O=C(Cl)c1cc(Cl)ccc1O ; Nc1cc([N+](=O)[O-])c(Cl)cc1F ; O=C(Nc1cc([N+](=O)[O-])c(Cl)cc1F)c1cc(Cl)ccc1O]

Compound 46 was synthesized according to the representative procedure (b). LC-MS (method 2): t_{R}=5.94 min, m/z (M+H)⁺=346.

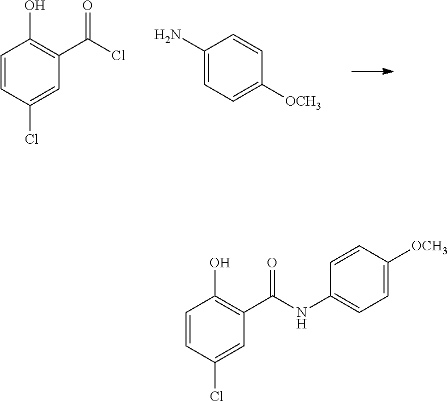 [SMILES: O=C(Cl)c1cc(Cl)ccc1O ; COc1ccc(N)cc1 ; COc1ccc(NC(=O)c2cc(Cl)ccc2O)cc1]

Compound 49 was synthesized according to the representative procedure (b). LC-MS (method 2): t_{R}=5.51 min, m/z (M+H)⁺=278. ¹H NMR (400 MHz, DMSO-d₆) δ 12.03 (s, 1H), 10.39 (s, 1H), 7.96 (d, J=2.7 Hz, 1H), 7.61-7.52 (m, 2H), 7.43 (dd, J=8.8, 2.7 Hz, 1H), 7.00-6.88 (m, 3H), 3.73 (s, 3H).

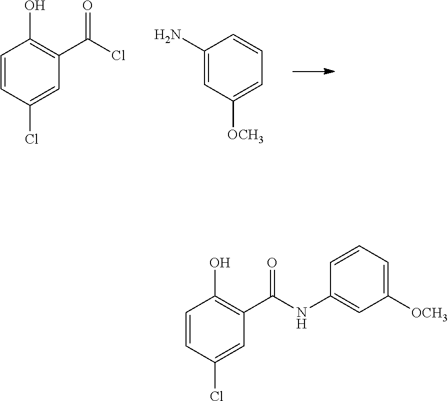 [SMILES: O=C(Cl)c1cc(Cl)ccc1O ; COc1cccc(N)c1 ; COc1cccc(NC(=O)c2cc(Cl)ccc2O)c1]

Compound 50 was synthesized according to the representative procedure (b). LC-MS (method 2): t_{R}=5.59 min, m/z (M+H)⁺=278.

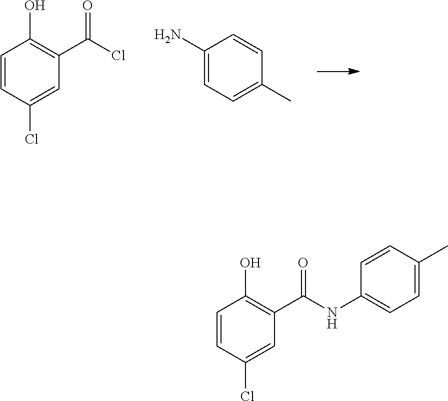

Compound 53 was synthesized according to the representative procedure (b). LC-MS (method 2): t_{R}=5.91 min, m/z (M+H)⁺=262.

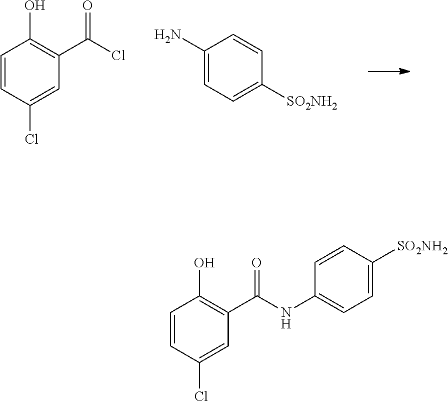 [SMILES: O=C(Cl)c1cc(Cl)ccc1O ; Nc1ccc(S(N)(=O)=O)cc1 ; NS(=O)(=O)c1ccc(NC(=O)c2cc(Cl)ccc2O)cc1]

Compound 38 was synthesized according to the representative procedure (b). LC-MS (method 2): t_{R}=4.36 min, m/z (M+H)⁺=327.

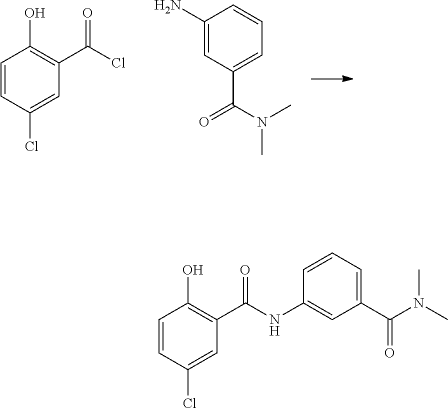 [SMILES: O=C(Cl)c1cc(Cl)ccc1O ; CN(C)C(=O)c1cccc(N)c1 ; CN(C)C(=O)c1cccc(NC(=O)c2cc(Cl)ccc2O)c1]

Compound 43 was synthesized according to the representative procedure (b). LC-MS (method 2): t_{R}=4.69 min, m/z (M+H)⁺=319. ¹H NMR (400 MHz, DMSO-d₆) δ 11.69 (s, 1H), 10.45 (s, 1H), 7.90 (d, J=2.6 Hz, 1H), 7.78-7.66 (m, 2H), 7.48-7.35 (m, 2H), 7.13 (dt, J=7.6, 1.4 Hz, 1H), 6.99 (dd, J=8.8, 1.4 Hz, 1H), 2.93 (d, J=23.8 Hz, 6H).

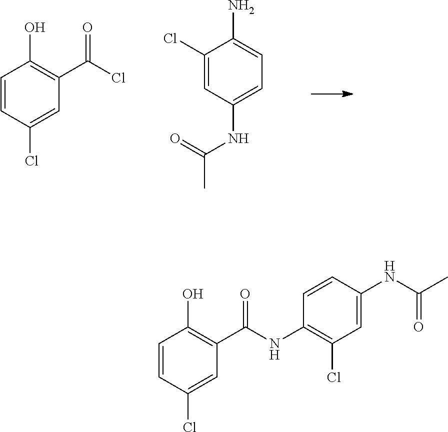 [SMILES: O=C(Cl)c1cc(Cl)ccc1O ; CC(=O)Nc1ccc(N)c(Cl)c1 ; CC(=O)Nc1ccc(NC(=O)c2cc(Cl)ccc2O)c(Cl)c1]

Compound 42 was synthesized according to the representative procedure (b). LC-MS (method 2): t_{R}=4.72 min, m/z (M+H)⁺=340. ¹H NMR (400 MHz, DMSO-d₆) δ 12.25 (s, 1H), 10.91 (s, 1H), 10.10 (s, 1H), 8.19 (d, J=8.9 Hz, 1H), 7.93 (dd, J=8.1, 2.5 Hz, 2H), 7.42 (ddd, J=18.7, 8.9, 2.5 Hz, 2H), 7.00 (d, J=8.8 Hz, 1H), 2.02 (s, 3H).

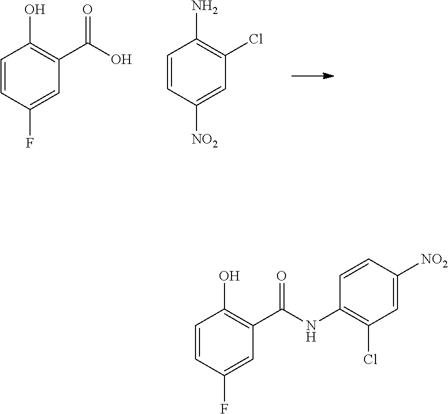 [SMILES: O=C(O)c1cc(F)ccc1O ; Nc1ccc([N+](=O)[O-])cc1Cl ; O=C(Nc1ccc([N+](=O)[O-])cc1Cl)c1cc(F)ccc1O]

Compound 5 was synthesized according to the representative procedure (a). LC-MS (method 2): t_{R}=5.65 min, m/z (M+H)⁺=311. ¹H NMR (400 MHz, DMSO-d₆) δ 12.23 (s, 1H), 11.58 (s, 1H), 8.81 (d, J=9.3 Hz, 1H), 8.41 (d, J=2.6 Hz, 1H), 8.27 (dd, J=9.2, 2.7 Hz, 1H), 7.70 (dd, J=9.6, 3.3 Hz, 1H), 7.35 (ddd, J=9.2, 7.8, 3.4 Hz, 1H), 7.05 (dd, J=9.0, 4.5 Hz, 1H).

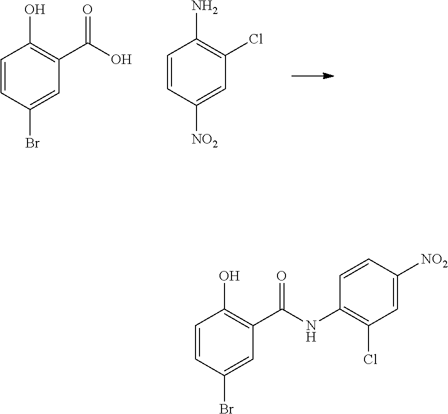 [SMILES: O=C(O)c1cc(Br)ccc1O ; Nc1ccc([N+](=O)[O-])cc1Cl ; O=C(Nc1ccc([N+](=O)[O-])cc1Cl)c1cc(Br)ccc1O]

Compound 6 was synthesized according to the representative procedure (a). LC-MS (method 2): t_{R}=6.03 min, m/z (M+H)⁺=372.

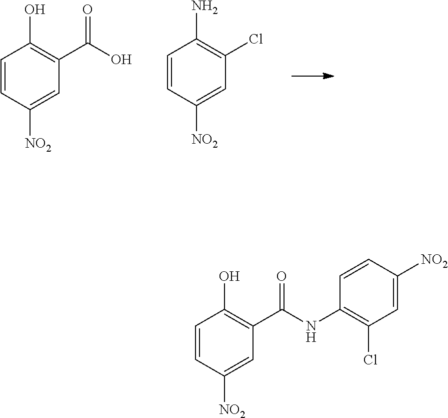 [SMILES: O=C(O)c1cc([N+](=O)[O-])ccc1O ; Nc1ccc([N+](=O)[O-])cc1Cl ; O=C(Nc1ccc([N+](=O)[O-])cc1Cl)c1cc([N+](=O)[O-])ccc1O]

Compound 12 was synthesized according to the representative procedure (a). LC-MS (method 2): t_{R}=5.59 min, m/z (M+H)⁺=338. ¹H NMR (400 MHz, DMSO-d₆) δ 12.69 (s, 1H), 8.84 (d, J=9.2 Hz, 1H), 8.78 (d, J=3.1 Hz, 1H), 8.40 (d, J=2.6 Hz, 1H), 8.26 (dd, J=9.3, 2.7 Hz, 1H), 8.16 (dd, J=9.3, 3.1 Hz, 1H), 6.91 (d, J=9.2 Hz, 1H).

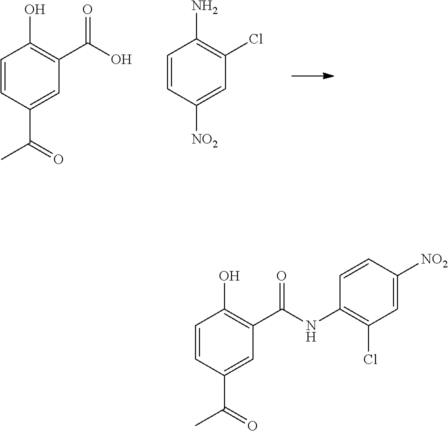 [SMILES: CC(=O)c1ccc(O)c(C(=O)O)c1 ; Nc1ccc([N+](=O)[O-])cc1Cl ; CC(=O)c1ccc(O)c(C(=O)Nc2ccc([N+](=O)[O-])cc2Cl)c1]

Compound 13 was synthesized according to the representative procedure (a). LC-MS (method2): t_{R}=5.33 min, m/z (M+H)⁺=335. ¹H NMR (400 MHz, DMSO-d₆) δ 13.11 (s, 1H), 11.61 (s, 1H), 8.82 (d, J=9.2 Hz, 1H), 8.61 (d, J=2.4 Hz, 1H), 8.42 (d, J=2.7 Hz, 1H), 8.28 (dd, J=9.3, 2.7 Hz, 1H), 8.02 (dd, J=8.6, 2.3 Hz, 1H), 7.09 (d, J=8.6 Hz, 1H), 2.52 (s, 3H).

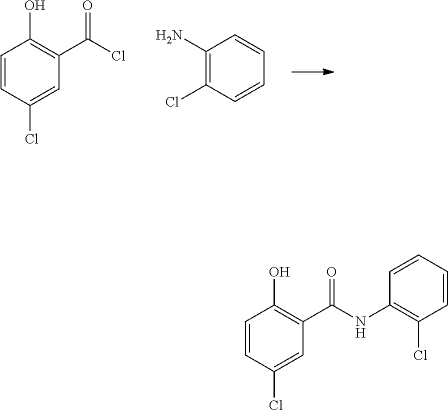 [SMILES: O=C(Cl)c1cc(Cl)ccc1O ; Nc1ccccc1Cl ; O=C(Nc1ccccc1Cl)c1cc(Cl)ccc1O]

Compound 23: 5-chloro-2-hydroxybenzoyl chloride (0.150 g, 0.784 mmol) and 2-chloroaniline (0.041 mL, 0.392 mmol) were suspended in pyridine (1.5 mL, 18.55 mmol) and stirred at room temperature for 5 h. The reaction mixture was then diluted with EtOAc (20 mL), washed with 10% CuSO₄ solution (20 mL), washed with 1N HCl solution, separated from aqueous layer, dried and concentrated to give the crude product which was purified by reverse phase HPLC. LC-MS (method 2): t_{R}=5.69 min, m/z (M+H)⁺=283. ¹H NMR (400 MHz, DMSO-d₆) δ 12.28 (s, 1H), 10.92 (s, 1H), 8.36 (dd, J=8.2, 1.6 Hz, 1H), 7.95 (d, J=2.8 Hz, 1H), 7.53 (dd, J=8.0, 1.5 Hz, 1H), 7.47 (dd, J=8.8, 2.8 Hz, 1H), 7.36 (ddd, J=8.4, 7.5, 1.5 Hz, 1H), 7.16 (td, J=7.7, 1.6 Hz, 1H), 7.03 (d, J=8.8 Hz, 1H).

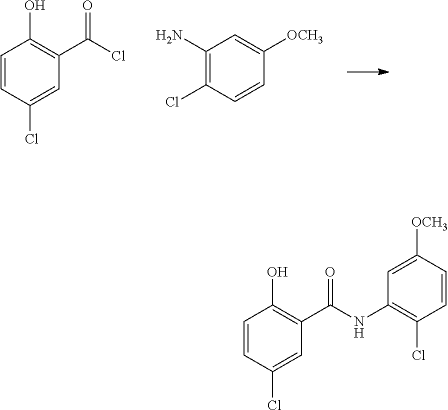 [SMILES: O=C(Cl)c1cc(Cl)ccc1O ; COc1ccc(Cl)c(N)c1 ; COc1ccc(Cl)c(NC(=O)c2cc(Cl)ccc2O)c1]

Compound 52 was synthesized according to the representative procedure (b). LC-MS (method 2): t_{R}=5.76 min, m/z (M+H)⁺=313. ¹H NMR (400 MHz, DMSO-d₆) δ 12.49-12.13 (m, 1H), 10.99 (s, 1H), 8.10 (d, J=3.0 Hz, 1H), 7.94 (d, J=2.8 Hz, 1H), 7.50-7.38 (m, 2H), 7.03 (d, J=8.7 Hz, 1H), 6.74 (dd, J=8.9, 3.0 Hz, 1H), 3.75 (s, 3H).

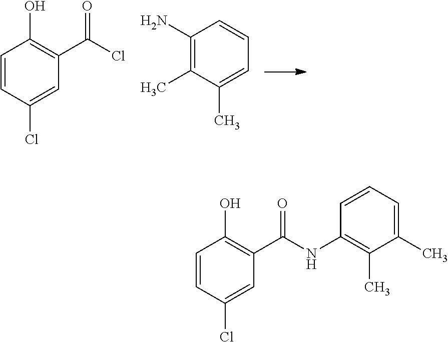

Compound 51: 5-chloro-2-hydroxybenzoyl chloride (0.158 g, 0.825 mmol) and 2,3-dimethylaniline (0.050 g, 0.413 mmol) were suspended in pyridine (1.5 mL, 18.55 mmol) and stirred at room temperature for 5 h. The reaction mixture was then diluted with EtOAc (20 mL), washed with 10% CuSO₄ solution (20 mL), washed with 1N HCl solution, separated from aqueous layer, dried and concentrated to give the crude product which was purified by reverse phase HPLC. LC-MS (method 2): t_{R}=5.80 min, m/z (M+H)⁺=276. ¹H NMR (400 MHz, DMSO-d₆) δ 12.26 (s, 1H), 10.34 (s, 1H), 8.01 (d, J=2.7 Hz, 1H), 7.49-7.41 (m, 2H), 7.13-6.95 (m, 3H), 2.26 (s, 3H), 2.11 (s, 3H).

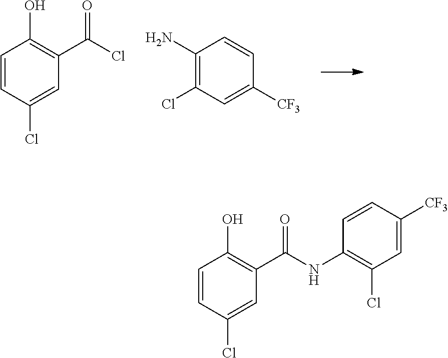 [SMILES: O=C(Cl)c1cc(Cl)ccc1O ; Nc1ccc(C(F)(F)F)cc1Cl ; O=C(Nc1ccc(C(F)(F)F)cc1Cl)c1cc(Cl)ccc1O]

Compound 40 was synthesized according to the representative procedure (b). LC-MS (Basic NEG Standard Gradient) (4% to 100% Acetonitrile (0.1% NH₄OH) over 3 minutes): t_{R}=2.90 min, m/z (M+H)⁺=351. ¹H NMR (400 MHz, DMSO-d₆) δ 12.37 (s, 1H), 11.42 (s, 1H), 8.71 (dd, J=8.8, 0.9 Hz, 1H), 7.98-7.90 (m, 2H), 7.75 (ddt, J=8.8, 1.6, 0.8 Hz, 1H), 7.46 (dd, J=8.7, 2.9 Hz, 1H), 7.02 (d, J=8.8 Hz, 1H).

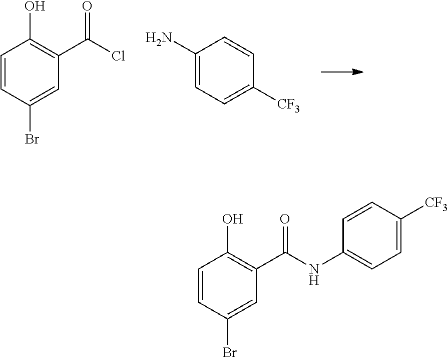 [SMILES: O=C(Cl)c1cc(Br)ccc1O ; Nc1ccc(C(F)(F)F)cc1 ; O=C(Nc1ccc(C(F)(F)F)cc1)c1cc(Br)ccc1O]

Compound 57 was synthesized according to the representative procedure (b). LC-MS (Basic NEG Standard Gradient) (4% to 100% Acetonitrile (0.1% NH₄OH) over 3 minutes): t_{R}=2.88 min. ¹H NMR (400 MHz, DMSO-d₆) δ 11.56 (s, 1H), 10.74 (s, 1H), 7.98-7.86 (m, 3H), 7.78-7.66 (m, 2H), 7.54 (dd, J=8.8, 2.6 Hz, 1H), 6.93 (d, J=8.7 Hz, 1H).

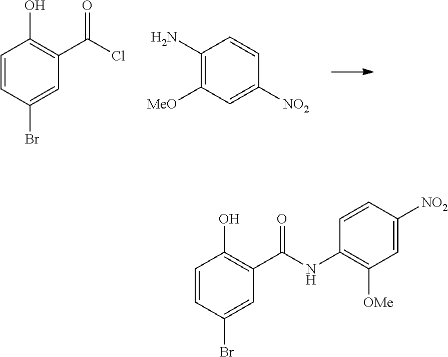 [SMILES: O=C(Cl)c1cc(Br)ccc1O ; COc1cc([N+](=O)[O-])ccc1N ; COc1cc([N+](=O)[O-])ccc1NC(=O)c1cc(Br)ccc1O]

Compound 61 was synthesized according to the representative procedure (b). LC-MS (method 2): t_{R}=5.86 min, m/z (M+H)⁺=368.

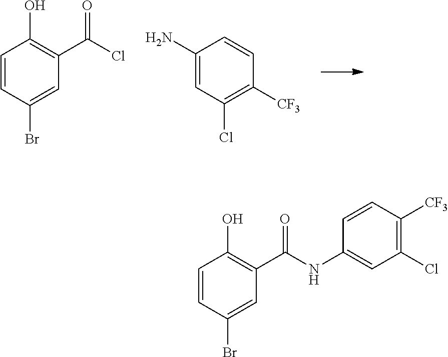 [SMILES: O=C(Cl)c1cc(Br)ccc1O ; Nc1ccc(C(F)(F)F)c(Cl)c1 ; O=C(Nc1ccc(C(F)(F)F)c(Cl)c1)c1cc(Br)ccc1O]

Compound 58 was synthesized according to the representative procedure (b). LC-MS (method 2): t_{R}=6.70 min, m/z (M+H)⁺=395. ¹H NMR (400 MHz, DMSO-d₆) δ 11.42 (s, 1H), 10.71 (s, 1H), 8.12 (dt, J=1.6, 0.8 Hz, 1H), 7.92 (d, J=2.6 Hz, 1H), 7.88-7.77 (m, 2H), 7.56 (dd, J=8.8, 2.6 Hz, 1H), 6.95 (d, J=8.8 Hz, 1H).

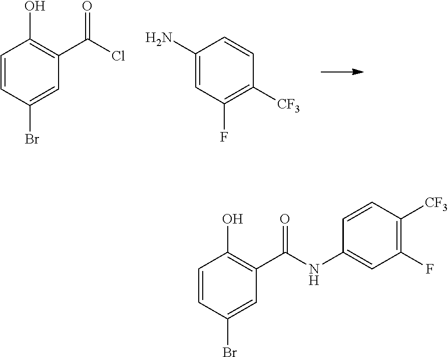 [SMILES: O=C(Cl)c1cc(Br)ccc1O ; Nc1ccc(C(F)(F)F)c(F)c1 ; O=C(Nc1ccc(C(F)(F)F)c(F)c1)c1cc(Br)ccc1O]

Compound 59 was synthesized according to the representative procedure (b). LC-MS (method 2): t_{R}=6.42 min, m/z (M+H)⁺=379.

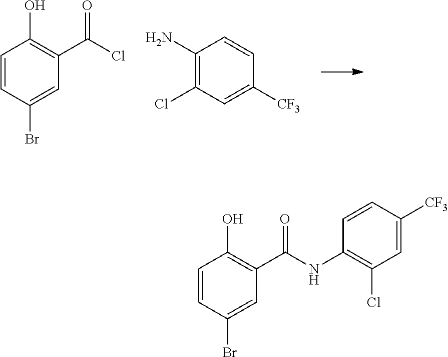 [SMILES: O=C(Cl)c1cc(Br)ccc1O ; Nc1ccc(C(F)(F)F)cc1Cl ; O=C(Nc1ccc(C(F)(F)F)cc1Cl)c1cc(Br)ccc1O]

Compound 60 was synthesized according to the representative procedure (b). LC-MS (method 2): t_{R}=6.54 min, m/z (M+H)⁺=395.

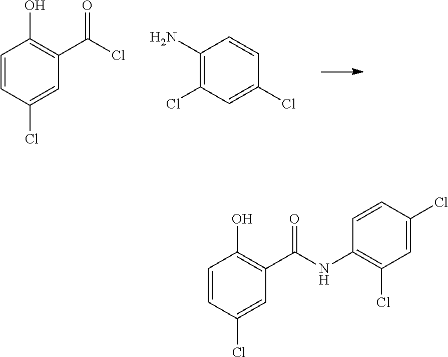 [SMILES: O=C(Cl)c1cc(Cl)ccc1O ; Nc1ccc(Cl)cc1Cl ; O=C(Nc1ccc(Cl)cc1Cl)c1cc(Cl)ccc1O]

Compound 44 was synthesized according to the representative procedure (b). LC-MS (Basic NEG Standard Gradient) (4% to 100% Acetonitrile (0.1% NH₄OH) over 3 minutes): t_{R}=2.77 min. ¹H NMR (400 MHz, DMSO-d₆) δ 12.32 (s, 1H), 11.20 (s, 1H), 8.41 (d, J=9.0 Hz, 1H), 7.92 (d, J=2.8 Hz, 1H), 7.70 (d, J=2.4 Hz, 1H), 7.44 (ddd, J=8.7, 2.6, 1.1 Hz, 2H), 7.00 (d, J=8.8 Hz, 1H).

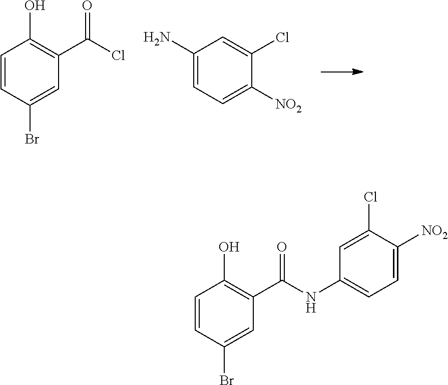 [SMILES: O=C(Cl)c1cc(Br)ccc1O ; Nc1ccc([N+](=O)[O-])c(Cl)c1 ; O=C(Nc1ccc([N+](=O)[O-])c(Cl)c1)c1cc(Br)ccc1O]

Compound 62 was synthesized according to the representative procedure (b). LC-MS (method 2): t_{R}=6.12 min, m/z (M+H)⁺=372.

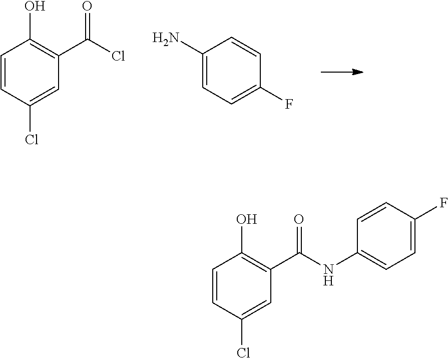 [SMILES: O=C(Cl)c1cc(Cl)ccc1O ; Nc1ccc(F)cc1 ; O=C(Nc1ccc(F)cc1)c1cc(Cl)ccc1O]

Compound 36 was synthesized according to the representative procedure (b). LC-MS (method 2): t_{R}=5.70 min, m/z (M+H)⁺=266.

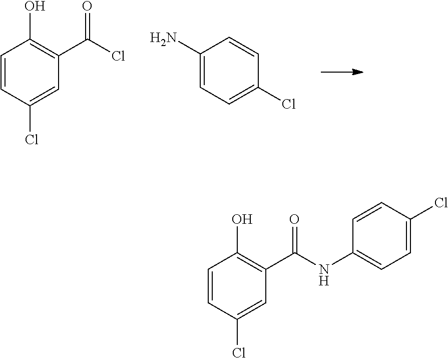 [SMILES: O=C(Cl)c1cc(Cl)ccc1O ; Nc1ccc(Cl)cc1 ; O=C(Nc1ccc(Cl)cc1)c1cc(Cl)ccc1O]

Compound 45 was synthesized according to the representative procedure (b). LC-MS (method 2): t_{R}=6.10 min, m/z (M+H)⁺=283.

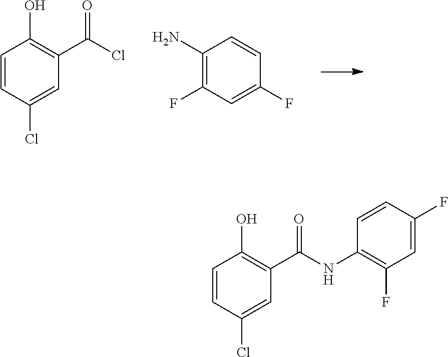 [SMILES: O=C(Cl)c1cc(Cl)ccc1O ; Nc1ccc(F)cc1F ; O=C(Nc1ccc(F)cc1F)c1cc(Cl)ccc1O]

Compound 34 was synthesized according to the representative procedure (b). LC-MS (method 2): t_{R}=5.59 min, m/z (M+H)⁺=284.

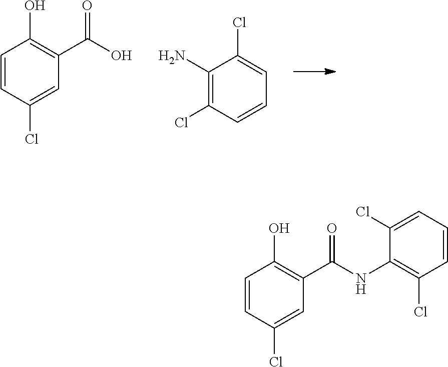 [SMILES: O=C(O)c1cc(Cl)ccc1O ; Nc1c(Cl)cccc1Cl ; O=C(Nc1c(Cl)cccc1Cl)c1cc(Cl)ccc1O]

Compound 47 was synthesized according to the representative procedure (a). LC-MS (method 2): t_{R}=5.65 min, m/z (M+H)⁺=317.

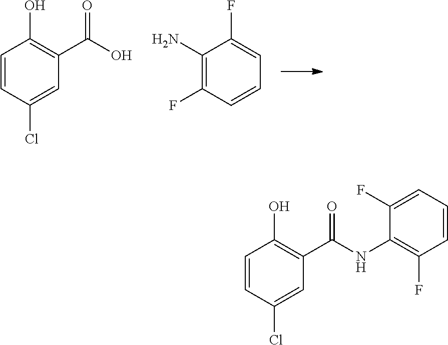 [SMILES: O=C(O)c1cc(Cl)ccc1O ; Nc1c(F)cccc1F ; O=C(Nc1c(F)cccc1F)c1cc(Cl)ccc1O]

Compound 48 was synthesized according to the representative procedure (a). LC-MS (method 2): t_{R}=5.32 min, m/z (M+H)⁺=284.

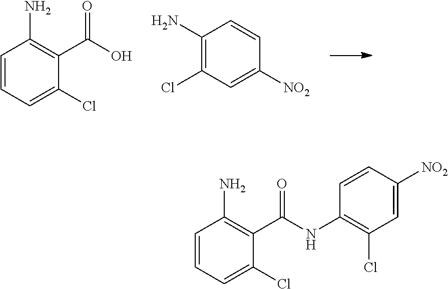 [SMILES: Nc1cccc(Cl)c1C(=O)O ; Nc1ccc([N+](=O)[O-])cc1Cl ; Nc1cccc(Cl)c1C(=O)Nc1ccc([N+](=O)[O-])cc1Cl]

Compound 18 was synthesized according to the representative procedure (a). LC-MS (method 2): t_{R}=5.56 min, m/z (M+H)⁺=326. ¹H NMR (400 MHz, DMSO-d₆) δ 10.49 (s, 1H), 8.37-8.31 (m, 1H), 8.23 (d, J=1.7 Hz, 2H), 7.08 (dd, J=8.3, 7.9 Hz, 1H), 6.71-6.58 (m, 2H), 5.47 (s, 2H).

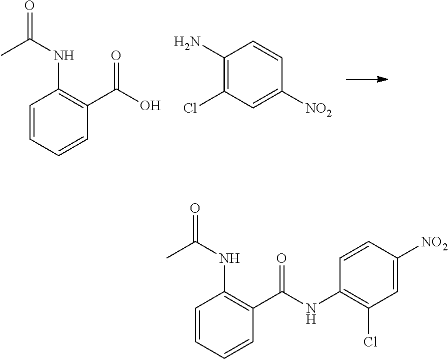 [SMILES: CC(=O)Nc1ccccc1C(=O)O ; Nc1ccc([N+](=O)[O-])cc1Cl ; CC(=O)Nc1ccccc1C(=O)Nc1ccc([N+](=O)[O-])cc1Cl]

Compound 19 was synthesized according to the representative procedure (a). LC-MS (Basic NEG Standard Gradient) (4% to 100% Acetonitrile (0.1% NH₄OH) over 3 minutes): t_{R}=2.96 min. m/z (M−H)⁻=332. ¹H NMR (400 MHz, DMSO-d₆) δ 8.63-8.57 (m, 1H), 8.41 (dd, J=8.7, 2.5 Hz, 1H), 8.11 (ddd, J=7.9, 1.6, 0.6 Hz, 1H), 8.04 (dd, J=8.7, 0.4 Hz, 1H), 7.88 (ddd, J=8.2, 7.2, 1.6 Hz, 1H), 7.69 (ddd, J=8.2, 1.2, 0.6 Hz, 1H), 7.55 (ddd, J=7.9, 7.2, 1.2 Hz, 1H), 2.11 (s, 3H).

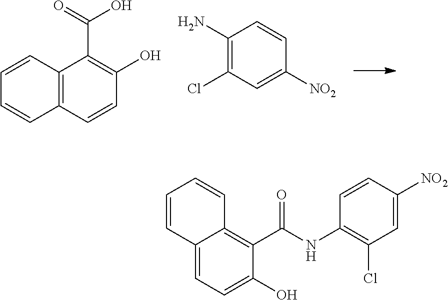 [SMILES: O=C(O)c1c(O)ccc2ccccc12 ; Nc1ccc([N+](=O)[O-])cc1Cl ; O=C(Nc1ccc([N+](=O)[O-])cc1Cl)c1c(O)ccc2ccccc12]

Compound 15 was synthesized according to the representative procedure (a). LC-MS (Basic NEG Standard Gradient) (4% to 100% Acetonitrile (0.1% NH₄OH) over 3 minutes): t_{R}=2.86 min. m/z (M−H)⁻=341. ¹H NMR (400 MHz, DMSO-d₆) δ 10.65 (d, J=58.7 Hz, 2H), 8.50 (d, J=8.8 Hz, 1H), 8.38 (d, J=2.6 Hz, 1H), 8.28 (dd, J=9.1, 2.6 Hz, 1H), 8.07 (d, J=8.6 Hz, 1H), 7.86 (dd, J=23.4, 8.5 Hz, 2H), 7.47 (ddd, J=8.5, 6.9, 1.4 Hz, 1H), 7.32 (ddd, J=8.1, 6.8, 1.2 Hz, 1H), 7.22 (d, J=8.9 Hz, 1H).

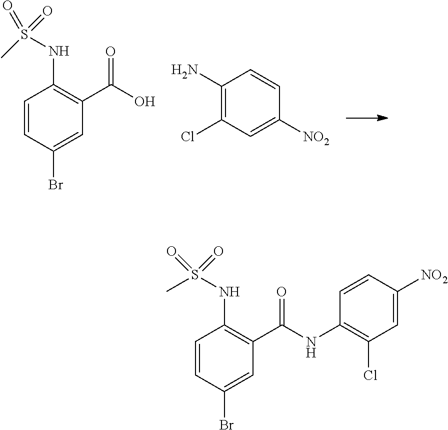 [SMILES: CS(=O)(=O)Nc1ccc(Br)cc1C(=O)O ; Nc1ccc([N+](=O)[O-])cc1Cl ; CS(=O)(=O)Nc1ccc(Br)cc1C(=O)Nc1ccc([N+](=O)[O-])cc1Cl]

Compound 20 was synthesized according to the representative procedure (a). LC-MS (Basic NEG Standard Gradient) (4% to 100% Acetonitrile (0.1% NH₄OH) over 3 minutes): t_{R}=2.59 min. m/z (M−H)⁻=446.

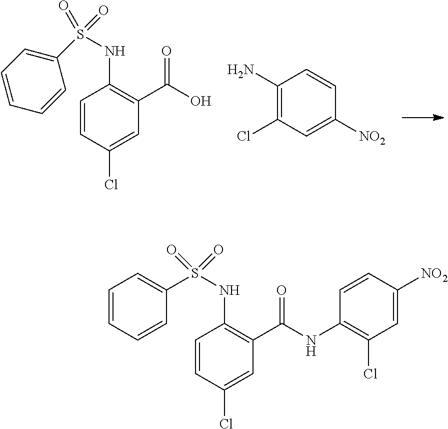 [SMILES: O=C(O)c1cc(Cl)ccc1NS(=O)(=O)c1ccccc1 ; Nc1ccc([N+](=O)[O-])cc1Cl ; O=C(Nc1ccc([N+](=O)[O-])cc1Cl)c1cc(Cl)ccc1NS(=O)(=O)c1ccccc1]

Compound 21 was synthesized according to the representative procedure (a). LC-MS (method 2): t_{R}=6.12 min, m/z (M+H)⁺=466.

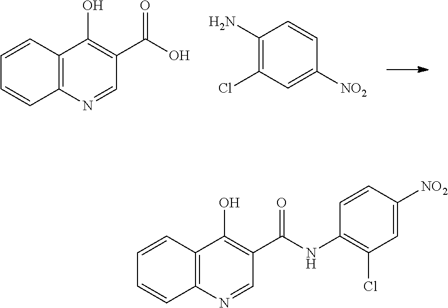 [SMILES: O=C(O)c1cnc2ccccc2c1O ; Nc1ccc([N+](=O)[O-])cc1Cl ; O=C(Nc1ccc([N+](=O)[O-])cc1Cl)c1cnc2ccccc2c1O]

Compound 68 was synthesized according to the representative procedure (a). LC-MS (method 2): t_{R}=5.21 min, m/z (M+H)⁺=344.

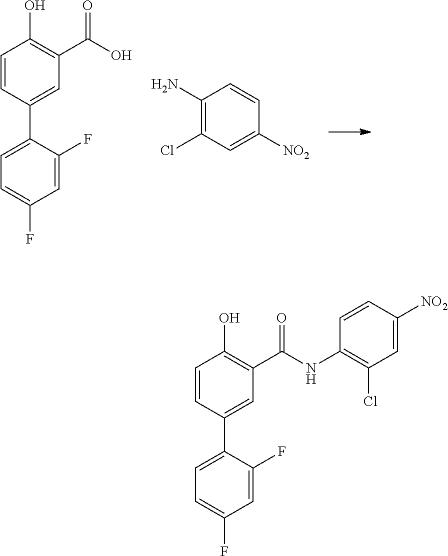 [SMILES: O=C(O)c1cc(-c2ccc(F)cc2F)ccc1O ; Nc1ccc([N+](=O)[O-])cc1Cl ; O=C(Nc1ccc([N+](=O)[O-])cc1Cl)c1cc(-c2ccc(F)cc2F)ccc1O]

Compound 16 was synthesized according to the representative procedure (a). LC-MS (Basic NEG Standard Gradient) (4% to 100% Acetonitrile (0.1% NH₄OH) over 3 minutes): t_{R}=3.00 min. m/z (M−H)⁻=403. ¹H NMR (400 MHz, DMSO-d₆) δ 12.46 (s, 1H), 8.84 (d, J=9.3 Hz, 1H), 8.41 (d, J=2.6 Hz, 1H), 8.27 (dd, J=9.3, 2.6 Hz, 1H), 8.14 (s, 1H), 7.57 (ddd, J=15.6, 10.4, 7.7 Hz, 2H), 7.33 (ddd, J=11.6, 9.2, 2.6 Hz, 1H), 7.21-7.07 (m, 2H).

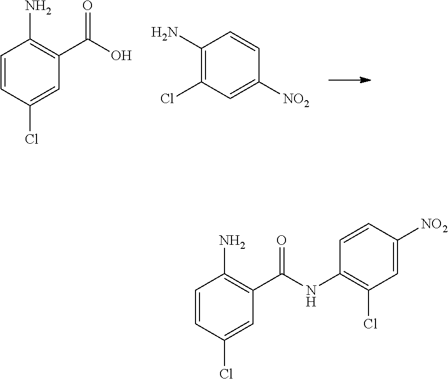 [SMILES: Nc1ccc(Cl)cc1C(=O)O ; Nc1ccc([N+](=O)[O-])cc1Cl ; Nc1ccc(Cl)cc1C(=O)Nc1ccc([N+](=O)[O-])cc1Cl]

Compound 17 was synthesized according to the representative procedure (a). LC-MS (Basic NEG Standard Gradient) (4% to 100% Acetonitrile (0.1% NH₄OH) over 3 minutes): t_{R}=3.31 min. m/z (M−H)⁻=324. ¹H NMR (400 MHz, DMSO-d₆) δ 8.36 (s, 1H), 8.20 (d, J=9.0 Hz, 1H), 7.95 (s, 1H), 7.75 (d, J=2.5 Hz, 2H), 7.25 (d, J=8.9 Hz, 1H), 6.79 (d, J=8.9 Hz, 2H).

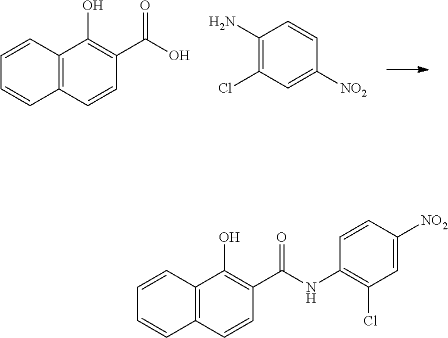 [SMILES: O=C(O)c1ccc2ccccc2c1O ; Nc1ccc([N+](=O)[O-])cc1Cl ; O=C(Nc1ccc([N+](=O)[O-])cc1Cl)c1ccc2ccccc2c1O]

Compound 14 was synthesized according to the representative procedure (a). LC-MS (Basic NEG Standard Gradient) (4% to 100% Acetonitrile (0.1% NH₄OH) over 3 minutes): t_{R}=2.50 min. m/z (M−H)⁻=341. ¹H NMR (400 MHz, DMSO-d₆) δ 13.01 (s, 1H), 11.68 (s, 1H), 8.46-8.38 (m, 2H), 8.34 (d, J=8.3 Hz, 1H), 8.27 (dd, J=9.1, 2.6 Hz, 1H), 8.04 (d, J=8.9 Hz, 1H), 7.88 (d, J=8.1 Hz, 1H), 7.64 (t, J=7.5 Hz, 1H), 7.56 (t, J=7.7 Hz, 1H), 7.44 (d, J=9.6 Hz, 1H).

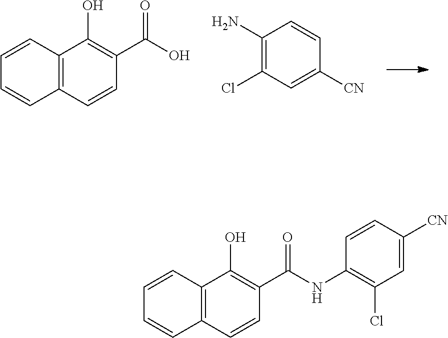 [SMILES: O=C(O)c1ccc2ccccc2c1O ; N#Cc1ccc(N)c(Cl)c1 ; N#Cc1ccc(NC(=O)c2ccc3ccccc3c2O)c(Cl)c1]

Compound 64 was synthesized according to the representative procedure (a). LC-MS (Basic NEG Standard Gradient) (4% to 100% Acetonitrile (0.1% NH₄OH) over 3 minutes): t_{R}=2.48 min. m/z (M−H)⁻=321. ¹H NMR (400 MHz, DMSO-d₆) δ 13.13 (s, 1H), 8.36-8.24 (m, 2H), 8.18 (d, J=2.1 Hz, 1H), 8.01 (d, J=8.7 Hz, 1H), 7.91-7.83 (m, 2H), 7.61 (d, J=7.8 Hz, 1H), 7.55 (d, J=8.6 Hz, 1H), 7.40 (s, 1H).

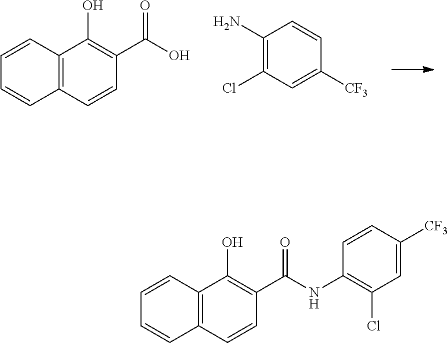 [SMILES: O=C(O)c1ccc2ccccc2c1O ; Nc1ccc(C(F)(F)F)cc1Cl ; O=C(Nc1ccc(C(F)(F)F)cc1Cl)c1ccc2ccccc2c1O]

Compound 63 was synthesized according to the representative procedure (a). LC-MS (Basic NEG Standard Gradient) (4% to 100% Acetonitrile (0.1% NH₄OH) over 3 minutes): t_{R}=2.93 min. m/z (M−H)⁻=364. ¹H NMR (400 MHz, DMSO-d₆) δ 13.31 (s, 1H), 11.17 (s, 1H), 8.31 (d, J=8.3 Hz, 1H), 8.18-8.11 (m, 1H), 8.08-7.98 (m, 2H), 7.89 (d, J=8.1 Hz, 1H), 7.79 (dd, J=8.6, 2.1 Hz, 1H), 7.64 (t, J=7.5 Hz, 1H), 7.56 (t, J=7.7 Hz, 1H), 7.45 (d, J=9.0 Hz, 1H).

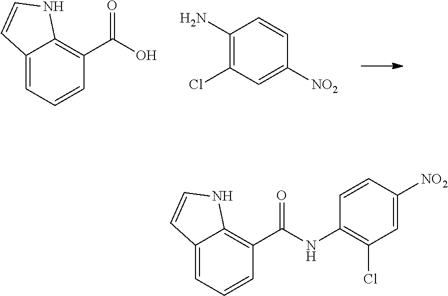 [SMILES: O=C(O)c1cccc2cc[nH]c12 ; Nc1ccc([N+](=O)[O-])cc1Cl ; O=C(Nc1ccc([N+](=O)[O-])cc1Cl)c1cccc2cc[nH]c12]

Compound 22 was synthesized according to the representative procedure (a). LC-MS (Basic NEG Standard Gradient) (4% to 100% Acetonitrile (0.1% NH₄OH) over 3 minutes): t_{R}=3.32 min. m/z (M−H)⁻=314. ¹H NMR (400 MHz, DMSO-d₆) δ 11.25 (s, 1H), 10.26 (s, 1H), 8.41 (d, J=2.5 Hz, 1H), 8.28 (d, J=9.1 Hz, 1H), 8.12 (d, J=8.9 Hz, 1H), 7.85 (dd, J=13.1, 7.6 Hz, 2H), 7.37 (t, J=2.7 Hz, 1H), 7.15 (t, J=7.7 Hz, 1H), 6.53 (dd, J=3.2, 1.7 Hz, 1H).

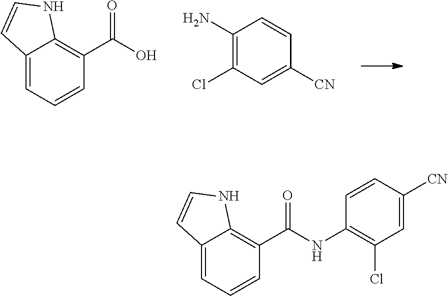 [SMILES: O=C(O)c1cccc2cc[nH]c12 ; N#Cc1ccc(N)c(Cl)c1 ; N#Cc1ccc(NC(=O)c2cccc3cc[nH]c23)c(Cl)c1]

Compound 67 was synthesized according to the representative procedure (a). LC-MS (Basic NEG Standard Gradient) (4% to 100% Acetonitrile (0.1% NH₄OH) over 3 minutes): t_{R}=3.28 min. m/z (M−H)⁻=294. ¹H NMR (400 MHz, DMSO-d₆) δ 11.21 (s, 1H), 10.20 (s, 1H), 8.16 (s, 1H), 7.97 (s, 1H), 7.90-7.78 (m, 3H), 7.36 (t, J=2.6 Hz, 1H), 7.13 (t, J=7.7 Hz, 1H), 6.52 (dd, J=3.1, 1.6 Hz, 1H).

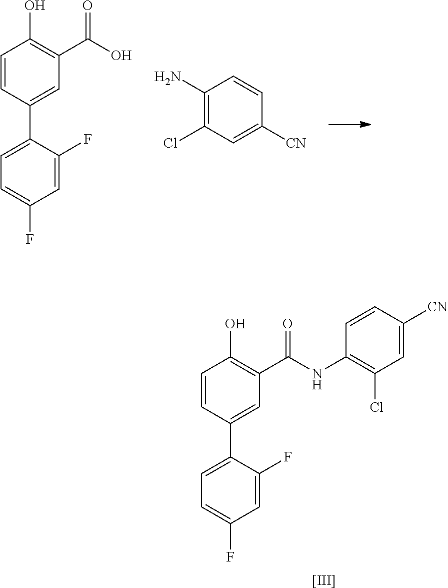

Compound 66 was synthesized according to the representative procedure (a). LC-MS (Basic NEG Standard Gradient) (4% to 100% Acetonitrile (0.1% NH₄OH) over 3 minutes): t_{R}=2.98 min. m/z (M−H)⁻=383.

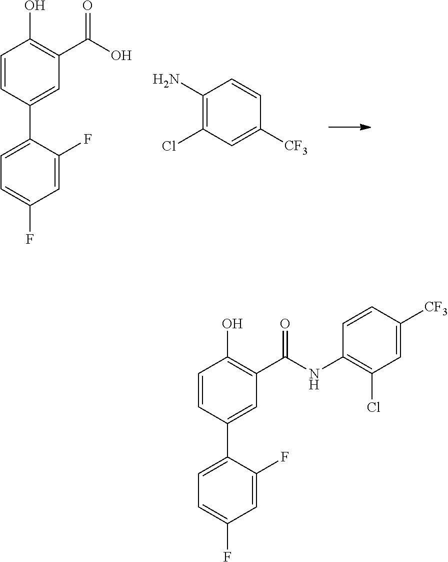 [SMILES: O=C(O)c1cc(-c2ccc(F)cc2F)ccc1O ; Nc1ccc(C(F)(F)F)cc1Cl ; O=C(Nc1ccc(C(F)(F)F)cc1Cl)c1cc(-c2ccc(F)cc2F)ccc1O]

Compound 65 was synthesized according to the representative procedure (a). LC-MS (Basic NEG Standard Gradient) (4% to 100% Acetonitrile (0.1% NH₄OH) over 3 minutes): t_{R}=3.33 min. m/z (M−H)⁻=426.

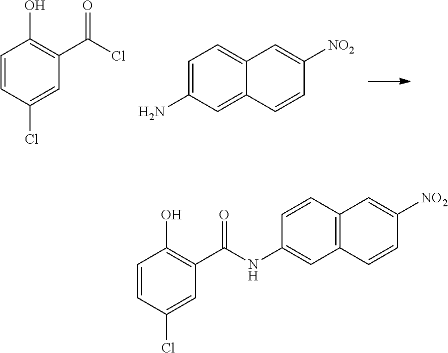 [SMILES: O=C(Cl)c1cc(Cl)ccc1O ; Nc1ccc2cc([N+](=O)[O-])ccc2c1 ; O=C(Nc1ccc2cc([N+](=O)[O-])ccc2c1)c1cc(Cl)ccc1O]

Compound 55 was synthesized according to the representative procedure (a). LC-MS (method 2): t_{R}=6.13 min, m/z (M+H)⁺=343. ¹H NMR (400 MHz, DMSO-d₆) δ 11.64 (s, 1H), 11.22 (s, 1H), 8.92 (dd, J=2.4, 0.6 Hz, 1H), 8.60 (d, J=2.0 Hz, 1H), 8.29-8.22 (m, 1H), 8.18 (dd, J=9.1, 2.3 Hz, 1H), 8.13-8.05 (m, 1H), 7.88 (td, J=4.1, 2.1 Hz, 2H), 7.43 (dd, J=8.8, 2.8 Hz, 1H), 6.98 (d, J=8.8 Hz, 1H).

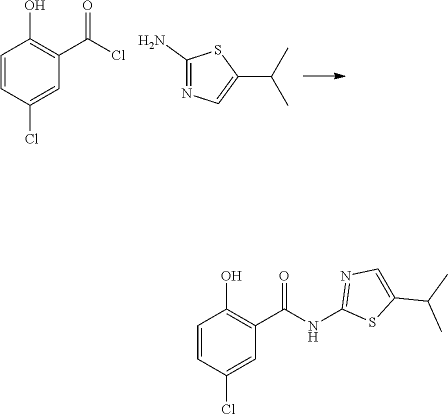

Compound 28: A mixture of 5-chloro-2-hydroxybenzoyl chloride (0.057 g, 0.3 mmol), 5-isopropylthiazol-2-amine (0.043 g, 0.300 mmol) and 200 ul Hunig's base in 2 mL of DCM was stirred at room temperature overnight. Solvent was removed and the residue was dissolved in 1 mL of DMSO. The solution was then subjected to reverse phase HPLC. LC-MS (method 2): t_{R}=5.76 min, m/z (M+H)⁺=297.

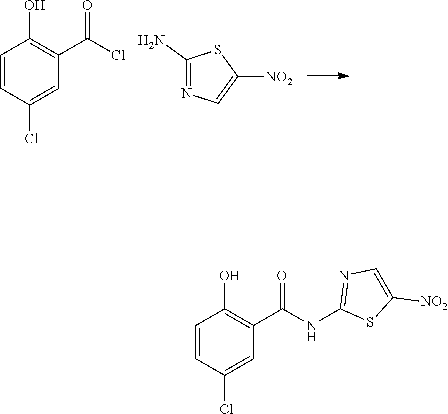

Compound 29: A mixture of 5-chloro-2-hydroxybenzoyl chloride (57.3 mg, 300 μmol), 5-nitrothiazol-2-amine (43.5 mg, 300 μmol), and 200 ul of Hunig's base in 1 mL of DMA and 1 mL of DCM was stirred at room temperature overnight. Solvent was then removed, and the crude product was purified by reverse phase HPLC. LC-MS (method 2): t_{R}=5.37 min, m/z (M+H)⁺=300.

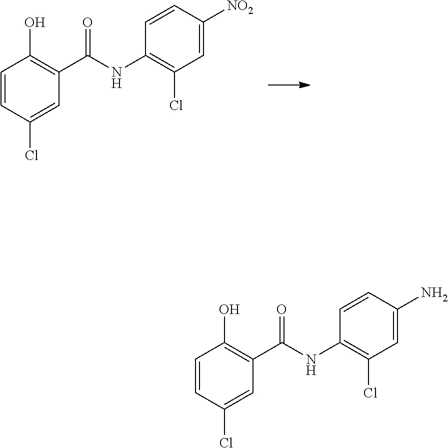 [SMILES: O=C(Nc1ccc([N+](=O)[O-])cc1Cl)c1cc(Cl)ccc1O ; Nc1ccc(NC(=O)c2cc(Cl)ccc2O)c(Cl)c1]

Compound 31: 5-chloro-N-(2-chloro-4-nitrophenyl)-2-hydroxybenzamide (1 g, 3.06 mmol) was dissolved in 20 mL of EtOH. Tin Chloride dihydrate (SnCl₂·2H₂O) was added as one portion (3.45 g, 15.29 mmol). The reaction mixture was then refluxed for 6 hours. After this time, the reaction was cooled to room temperature and then to a further 0° C. Saturated aqueous NaHCO₃ was added with stirring until pH >7. EtOAc (100 ml) was added and layers were separated. The aqueous layer was washed again with EtOAC (100 ml). EtOAc layers were combined, dried over MgSO₄, filtered and concentrated. The resulting crude product was purified by normal phase column chromatography with a solvent gradient (2% DCM:MeOH to 10% DCM:MeOH) to provide N-(4-amino-2-chlorophenyl)-5-chloro-2-hydroxybenzamide as a white solid (0.5 g, 1.68 mmol) in 55% yield. LC-MS (method 2): t_{R}=4.31 min, m/z (M+H)⁺=297.

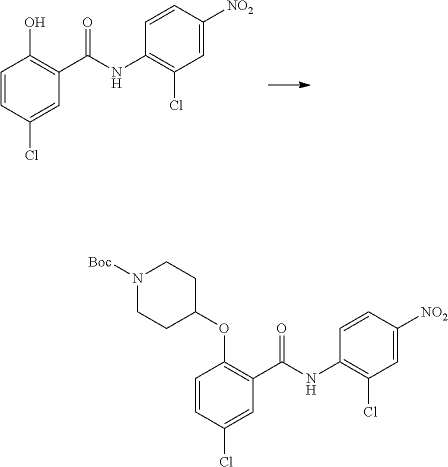 [SMILES: O=C(Nc1ccc([N+](=O)[O-])cc1Cl)c1cc(Cl)ccc1O ; CC(C)(C)OC(=O)N1CCC(Oc2ccc(Cl)cc2C(=O)Nc2ccc([N+](=O)[O-])cc2Cl)CC1]

Compound 4: To a solution of triphenylphosphine (118 mg, 450 μmol), 5-chloro-N-(2-chloro-4-nitrophenyl)-2-hydroxybenzamide (98 mg, 300 μmol), and tert-butyl 4-hydroxypiperidine-1-carboxylate (91 mg, 450 μmol) in 5 ml of THF, was added (E)-di-tert-butyl diazene-1,2-dicarboxylate (104 mg, 450 μmol). The mixture was then stirred at room temperature overnight after which the solution was concentrated, and the crude product was subjected to normal phase chromatography, gradient solvent system (20% EtOAc:Hexanes to 80% EtOAc:Hexanes), to yield tert-butyl 4-(4-chloro-2-((2-chloro-4-nitrophenyl)carbamoyl)phenoxy)piperidine-1-carboxylate.

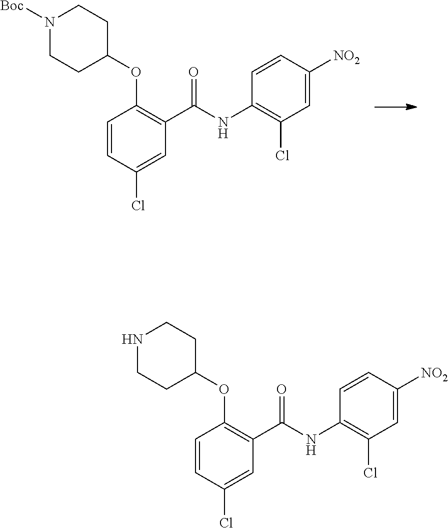 [SMILES: CC(C)(C)OC(=O)N1CCC(Oc2ccc(Cl)cc2C(=O)Nc2ccc([N+](=O)[O-])cc2Cl)CC1 ; O=C(Nc1ccc([N+](=O)[O-])cc1Cl)c1cc(Cl)ccc1OC1CCNCC1]

Compound 4 (cont'd): To a DCM solution (2 ml) of tert-butyl 4-(4-chloro-2-((2-chloro-4-nitrophenyl)carbamoyl)phenoxy)piperidine-1-carboxylate (0.035 g, 0.069 mmol) at 0° C., TFA was added slowly (5 equivalents). After 60 minutes, LC-MS showed complete conversion. The mixture was then concentrated (co-evaporated with DCE (3 ml), three times), and the resulting mixture was triturated with MeOH (2×1 mL). The precipitate was dried and provided 5-chloro-N-(2-chloro-4-nitrophenyl)-2-(piperidin-4-yloxy)benzamideproduct as white solid (0.015 g, 0.036 mmol, 52% yield). LC-MS (method 2): t_{R}=4.60 min, m/z (M+H)⁺=410.

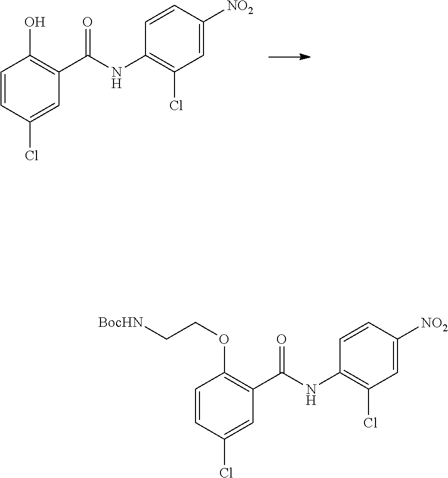 [SMILES: O=C(Nc1ccc([N+](=O)[O-])cc1Cl)c1cc(Cl)ccc1O ; CC(C)(C)OC(=O)NCCOc1ccc(Cl)cc1C(=O)Nc1ccc([N+](=O)[O-])cc1Cl]

Compound 3: To a solution of tert-butyl (2-hydroxyethyl)carbamate (72.5 mg, 450 μmol), 5-chloro-N-(2-chloro-4-nitrophenyl)-2-hydroxybenzamide (98 mg, 300 μmol), triphenylphosphine (118 mg, 450 μmol) in 5 mL of THF was added (E)-di-tert-butyl diazene-1,2-dicarboxylate (104 mg, 450 μmol), and the reaction was stirred at room temperature overnight. After that time, the reaction was concentrated, and the crude product was subjected to normal phase chromatography, gradient solvent system (20% EtOAc:Hexanes to 80% EtOAc:Hexanes), to yield tert-butyl (2-(4-chloro-2-((2-chloro-4-nitrophenyl)carbamoyl)phenoxy)ethyl)carbamate.

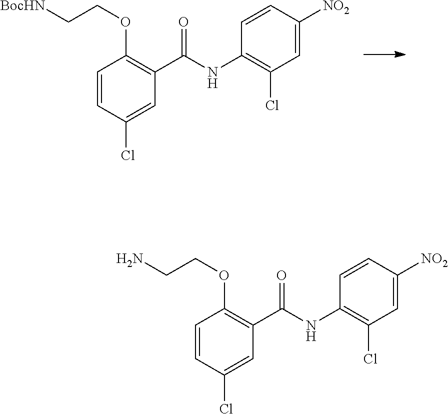 [SMILES: CC(C)(C)OC(=O)NCCOc1ccc(Cl)cc1C(=O)Nc1ccc([N+](=O)[O-])cc1Cl ; NCCOc1ccc(Cl)cc1C(=O)Nc1ccc([N+](=O)[O-])cc1Cl]

Compound 3 (cont'd): To a DCM solution (2 ml) of tert-butyl (2-(4-chloro-2-((2-chloro-4-nitrophenyl)carbamoyl)phenoxy)ethyl)carbamate (0.040 g, 0.085 mmol) at 0° C., TFA was added slowly (5 equivalents). After 60 min, LC-MS found complete conversion. The mixture was then concentrated (co-evaporated with DCE (3 ml), three times), and the resulting mixture was triturated with MeOH (2×1 mL). The precipitate was dried and provided 2-(2-aminoethoxy)-5-chloro-N-(2-chloro-4-nitrophenyl)benzamide as white solid (0.020 g, 0.054 mmol, 63% yield). LC-MS (method 2): t_{R}=4.20 min, m/z (M+H)⁺=371.

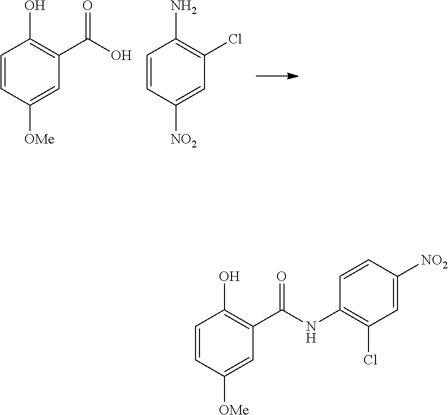

Compound 9: 2-hydroxy-5-methoxybenzoic acid (0.097 g, 0.579 mmol) and 2-chloro-4-nitroaniline (0.100 g, 0.579 mmol) were suspended in Xylene (2.90 ml) and heated to reflux. Then at that temperature Phosphorus trichloride (0.020 ml, 0.232 mmol) was added and then it was heated at 150° C. for 5 h. The reaction mixture was then cooled to room temperature, diluted with water, and stirred at RT for 30 min. The reaction mixture was then added dropwise into a saturated solution of NaHCO₃ and stirred overnight (pH=8). It was then extracted with EtOAc (2×50 ml). The organic layers were then combined, dried, filtered and concentrated to provide the crude product that was subjected to purification via reverse phase HPLC. ¹H NMR (400 MHz, DMSO-d6) δ 11.73 (s, 2H), 8.83 (dd, J=9.3, 1.3 Hz, 1H), 8.40 (d, J=2.6 Hz, 1H), 8.26 (ddd, J=9.4, 2.6, 1.2 Hz, 1H), 7.50 (d, J=3.3 Hz, 1H), 7.08 (dd, J=8.9, 3.2 Hz, 1H), 6.97 (d, J=8.9 Hz, 1H), 3.72 (d, J=1.3 Hz, 3H). LC-MS (method 2): t_{R}=5.55 min, m/z (M+H)⁺=323.

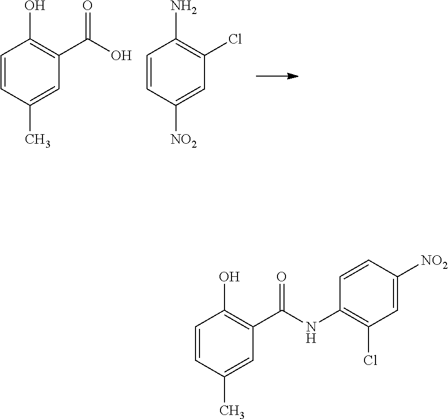

Compound 10: 2-hydroxy-5-methylbenzoic acid (0.088 g, 0.579 mmol) and 2-chloro-4-nitroaniline (0.100 g, 0.579 mmol) were suspended in Xylene (2.90 ml) and heated to reflux. Then at that temperature Phosphorus trichloride (0.020 ml, 0.232 mmol) was added and then it was heated at 150° C. for 5 h. The reaction mixture was then cooled to room temperature, diluted with water, and stirred at RT for 30 min. The reaction mixture was then added dropwise into a saturated solution of NaHCO₃ and stirred overnight (pH=8). It was then extracted with EtOAc (2×50 ml). The organic layers were then combined, dried, filtered and concentrated to provide the crude product that was subjected to purification via reverse phase HPLC. ¹H NMR (400 MHz, DMSO-d6) δ 11.90 (s, 1H), 11.61 (s, 1H), 8.83 (d, J=9.3 Hz, 1H), 8.40 (d, J=2.5 Hz, 1H), 8.26 (dd, J=9.3, 2.6 Hz, 1H), 7.81 (d, J=2.3 Hz, 1H), 7.26 (dd, J=8.3, 2.2 Hz, 1H), 6.93 (d, J=8.3 Hz, 1H), 2.25 (s, 3H). LC-MS (method 2): t_{R}=5.83 min, m/z (M+H)⁺=307. LC-MS (method 2): t_{R}=5.82 min, m/z (M+H)⁺=307.

### Compound P1

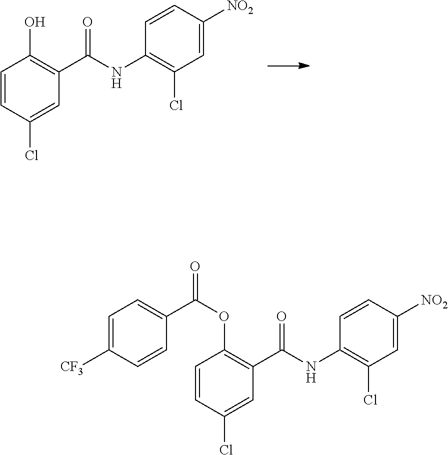 [SMILES: O=C(Nc1ccc([N+](=O)[O-])cc1Cl)c1cc(Cl)ccc1O ; O=C(Oc1ccc(Cl)cc1C(=O)Nc1ccc([N+](=O)[O-])cc1Cl)c1ccc(C(F)(F)F)cc1]

To a solution of 5-chloro-N-(2-chloro-4-nitrophenyl)-2-hydroxybenzamide (0.050 g, 0.153 mmol) in CH₂Cl₂ (0.510 ml), Hunig's base (0.027 ml, 0.153 mmol) and 4-(trifluoromethyl)benzoyl chloride (0.023 ml, 0.153 mmol) were added. The reaction was then stirred at room temperature for 15 min after which the reaction showed complete conversion. Saturated aqueous NH₄Cl (2 ml) was added to the reaction mixture, and the layers were separated. The organic layer was dried over MgSO₄, dried, filtered, and concentrated. It was then subjected to normal phase chromatography, gradient solvent system (20% EtOAc:Hexanes to 80% EtOAc:Hexanes), to yield 4-chloro-2-((2-chloro-4-nitrophenyl)carbamoyl)phenyl 4-(trifluoromethyl)benzoate (0.050 g, 0.100 mmol, 65.5% yield). LC-MS (method 2): t_{R}=6.85 min, m/z (M+H)⁺=500.

### Compound P3

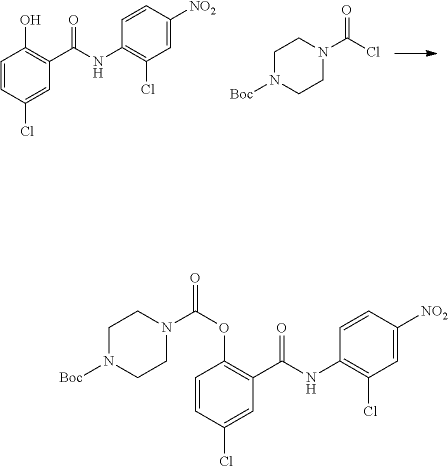 [SMILES: O=C(Nc1ccc([N+](=O)[O-])cc1Cl)c1cc(Cl)ccc1O ; CC(C)(C)OC(=O)N1CCN(C(=O)Cl)CC1 ; CC(C)(C)OC(=O)N1CCN(C(=O)Oc2ccc(Cl)cc2C(=O)Nc2ccc([N+](=O)[O-])cc2Cl)CC1]

5-chloro-N-(2-chloro-4-nitrophenyl)-2-hydroxybenzamide (0.500 g, 1.529 mmol) was dissolved in dry THF (7.64 ml). Et3N (0.256 ml, 1.834 mmol) and catalytic DMAP were added which was followed by tert-butyl 4-(chlorocarbonyl)piperazine-1-carboxylate (0.380 g, 1.529 mmol). The reaction was stirred at room temperature for 1 h after which the LC-MS showed complete conversion. The reaction was diluted with EtOAc (10 ml) and washed with 10 ml of saturated aqueous NH₄Cl. The organic layer was collected, dried, filtered and concentrated to give the crude product which was purified by flash column chromatography. A CH₂Cl₂:CH₃OH gradient was used (1-5% CH₃OH in CH₂Cl₂) to provide 1-(tert-butyl) 4-(4-chloro-2-((2-chloro-4-nitrophenyl)carbamoyl)phenyl) piperazine-1,4-dicarboxylate (0.720 g, 1.335 mmol, 87% yield). LC-MS (method 2): t_{R}=6.5 min, m/z (M+Na)⁺=561.

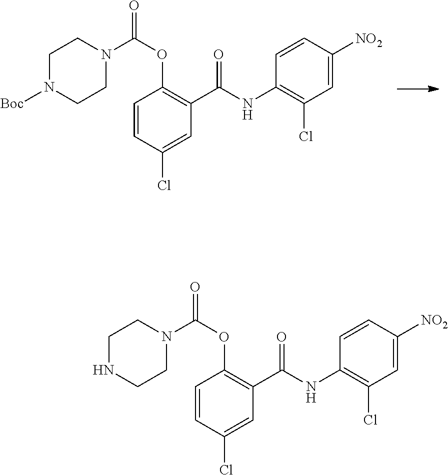 [SMILES: CC(C)(C)OC(=O)N1CCN(C(=O)Oc2ccc(Cl)cc2C(=O)Nc2ccc([N+](=O)[O-])cc2Cl)CC1 ; O=C(Nc1ccc([N+](=O)[O-])cc1Cl)c1cc(Cl)ccc1OC(=O)N1CCNCC1]

1-(tert-butyl) 4-(4-chloro-2-((2-chloro-4-nitrophenyl)carbamoyl)phenyl) piperazine-1,4-dicarboxylate (0.720 g, 1.335 mmol) was dissolved in dry EtOAc at room temperature (5 ml) and 4N HCl in dioxane (0.667 ml, 2.67 mmol) was added. The reaction was stirred at room temperature and was completed in around 4 h. The precipitated compound was filtered through a buchner funnel and washed with diethylether multiple times. The solid 4-chloro-2-((2-chloro-4-nitrophenyl)carbamoyl)phenyl piperazine-1-carboxylate (compound P3), HCl (0.590 g, 1.240 mmol, 93% yield) was collected and dried over vacuum overnight. ¹H NMR (400 MHz, DMSO-d6) δ 10.54 (s, 1H), 9.71 (s, 2H), 8.39 (d, J=2.6 Hz, 1H), 8.28 (dd, J=9.0, 2.6 Hz, 1H), 8.06 (d, J=9.0 Hz, 1H), 7.82 (d, J=2.6 Hz, 1H), 7.69 (dd, J=8.7, 2.6 Hz, 1H), 7.41 (d, J=8.7 Hz, 1H), 3.83 (s, 2H), 3.66 (s, 2H), 3.09 (s, 4H). LC-MS (method 2): t_{R}=4.07 min, m/z (M+H)⁺=439.

### Compound P5

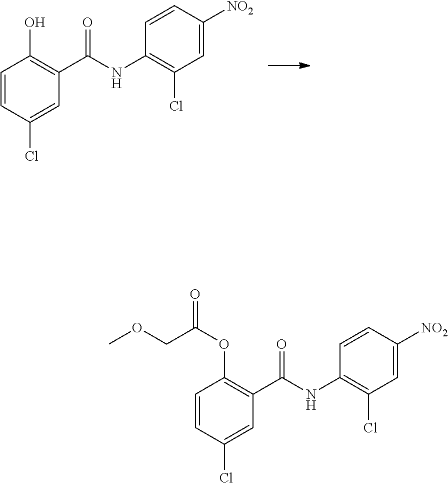 [SMILES: O=C(Nc1ccc([N+](=O)[O-])cc1Cl)c1cc(Cl)ccc1O ; COCC(=O)Oc1ccc(Cl)cc1C(=O)Nc1ccc([N+](=O)[O-])cc1Cl]

5-chloro-N-(2-chloro-4-nitrophenyl)-2-hydroxybenzamide (0.050 g, 0.153 mmol) was taken up in CH₂Cl₂ (0.510 ml) and cooled to 0° C. Hunig's base (0.027 ml, 0.153 mmol) and 2-methoxyacetyl chloride (0.014 ml, 0.153 mmol) were then added to the reaction mixture at 0° C. and the reaction was let to stir at that temperature for 20 min after which the reaction showed complete conversion to the product. The reaction was then stopped by adding saturated aqueous NaHCO₃ (2 ml). The organic layer was separated, dried over MgSO₄, filtered and concentrated. It was then subjected to normal phase column chromatography, gradient solvent system (20% EtOAc:Hexanes to 80% EtOAc:Hexanes), to yield 4-chloro-2-((2-chloro-4-nitrophenyl)carbamoyl)phenyl 2-methoxyacetate (0.045 g, 0.113 mmol, 73.8% yield). LC-MS (method 2): t_{R}=5.58 min, m/z (M+H)⁺=400.

### Compound P6

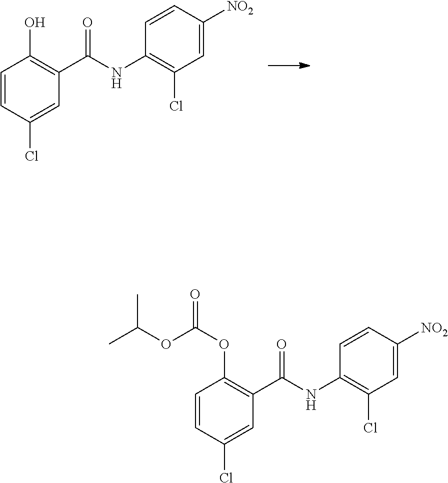 [SMILES: O=C(Nc1ccc([N+](=O)[O-])cc1Cl)c1cc(Cl)ccc1O ; CC(C)OC(=O)Oc1ccc(Cl)cc1C(=O)Nc1ccc([N+](=O)[O-])cc1Cl]

To a solution of 5-chloro-N-(2-chloro-4-nitrophenyl)-2-hydroxybenzamide (0.050 g, 0.153 mmol) in THF (1 ml) at 0° C., pyridine (0.014 ml, 0.168 mmol) and isopropyl carbonochloridate (1M in toluene) (0.168 ml, 0.168 mmol) were added. The reaction then warmed to room temperature and stirred for 20 min after which it showed complete conversion. Saturated aqueous NH₄Cl (2 ml) was added, and the layers were separated. The organic layer was dried over MgSO₄, filtered and concentrated. The reaction mixture was then subjected to normal phase column chromatography, gradient solvent system (20% EtOAc:Hexanes to 80% EtOAc:Hexanes), to yield 4-chloro-2-((2-chloro-4-nitrophenyl)carbamoyl)phenyl isopropyl carbonate (0.048 g, 0.116 mmol, 76% yield). LC-MS (method 2): t_{R}=6.52 min, m/z (M+H)⁺=413.

### Compound P9

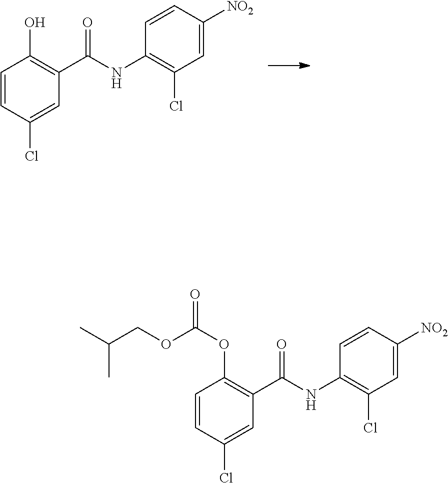 [SMILES: O=C(Nc1ccc([N+](=O)[O-])cc1Cl)c1cc(Cl)ccc1O ; CC(C)COC(=O)Oc1ccc(Cl)cc1C(=O)Nc1ccc([N+](=O)[O-])cc1Cl]

To a solution of 5-chloro-N-(2-chloro-4-nitrophenyl)-2-hydroxybenzamide (0.070 g, 0.214 mmol) in THF (1 ml) at 0° C., pyridine (0.019 ml, 0.235 mmol) and isobutyl carbonochloridate (0.031 ml, 0.235 mmol) were added and the reaction mixture was allowed to warm to room temperature. It was then stirred for 20 min after which the reaction showed complete disappearance of the starting materials. Saturated aqueous NH₄Cl (5 ml) was added and the layers were separated. The organic layer was then dried over MgSO₄, filtered and concentrated. It was then subjected to normal phase column chromatography, gradient solvent system (20% EtOAc:Hexanes to 80% EtOAc:Hexanes), to yield 4-chloro-2-((2-chloro-4-nitrophenyl)carbamoyl)phenyl isobutyl carbonate (0.075 g, 0.176 mmol, 82% yield). LC-MS (method 2): t_{R}=6.75 min, m/z (M+H)⁺=428.

### Example 2

### Cytopathic Effect Assay

Compounds 4, 6, 24, 27, 32, 39, and 59 of Table 1, above, and niclosamide were examined for activity against SARS-CoV-2. As SARS-CoV-2 can induce cell death after 48 to 72 h of infection, the activity of the compounds to protect against virus-induced cell death was evaluated in a cytopathic effect assay.

Vero-E6 cells selected for high ACE2 expression and were cultured in MEM/10% HI FBS supplemented with 0.5 μg/mL amphotericin B and passaged twice per week at 1:5 dilutions using trypsin. Briefly, cell culture media was aspirated, and cells were washed twice with PBS. 2 mL of trypsin was added for 1-2 min at room temperature and 10 mL of EMEM was added to wash flask to create a single cell suspension. Cells were spun at 800 RPM for 5 min. Supernatant was aspirated and cells were resuspended in fresh media for seeding into flasks or multi-well plates. Following this, cells were grown in EMEM, 10% FBS and 1% Pencillin/Streptomycin in T175 flasks and passaged at 95% confluency. Cells were then washed once with PBS and dissociated from the flask using TrypLE. Cells were counted prior to seeding.

Niclosamide was prepared as 10 mM solutions in DMSO and titrated for 10 points at 1:2. Then acoustically dispensed into assay plates at 90 nl/well. All other compounds were prepared as 10 mM solutions in DMSO, titrated for 10 points at 1:3, then acoustically dispensed into assay plates at 60 nl/well. These are referred to as Assay Ready Plates (ARPs) and are stored at −20° C.

Briefly, cell culture media (MEM, 1% Pen/Strep/GlutaMax, 1% HEPES, 2% HI FBS) was dispensed at 5 μl/well into ARPs and incubated at room temperature to allow for compound dissolution. Vero-E6 (selected for high ACE2 expression) was inoculated with SARS CoV-2 (USA_WA1/2020) at M.O.I. 0.002 in media and quickly dispensed into assay plates as 25 μl/well. The final cell density was 4000 cells/well. Assay plates were incubated for 72 hr at 37 C, 5% CO₂, 90% humidity. 30 μl/well of CellTiter-Glo (Promega #G7573) was dispensed into the assay plate. Following incubation at room temperature for 10 min the plates were sealed with a clear cover and surface decontaminated, and luminescence was read using a Perkin Elmer Envision (Waltham, MA) plate reader to measure cell viability. Raw data from each test well was normalized to the average signal of non-infected cells (Avg. Cells; 100% inhibition) and virus infected cells only (Avg. Virus; 0% inhibition) to calculate % inhibition of CPE using the following formula: % inhibition CPE=100*(Test Cmpd−Avg. Virus)/(Avg. Cells−Avg. Virus).

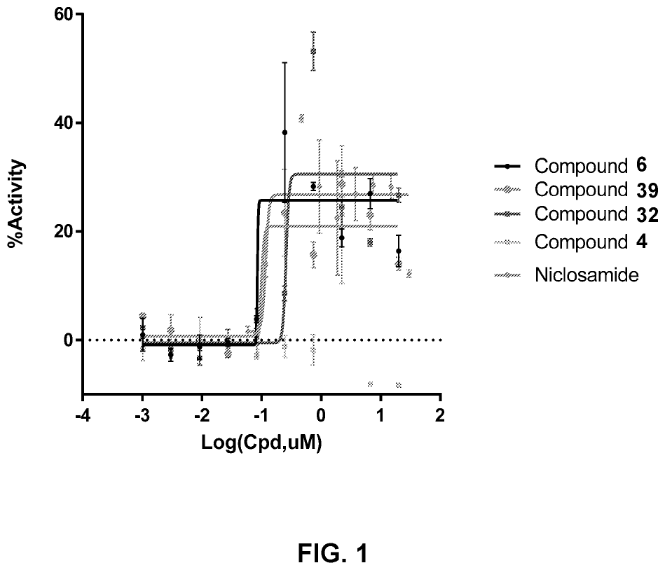

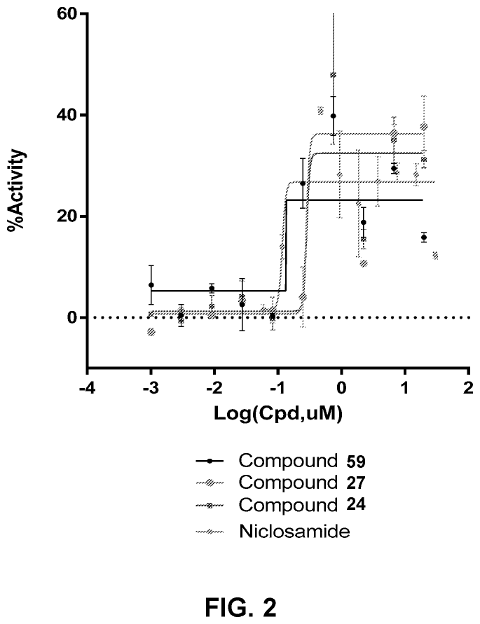

Infection of the Vero-E6 host cells with SARS-CoV-2 strain (USA_WA1/2020) at MOI of 0.002, which resulted in approximately 5% cell viability 72 h post infection. Concentration response rescue curves are shown in FIGS. 1 and 2. Using this protocol, niclosamide showed an IC₅₀ of around 112 nM at an efficacy of 30%. Compound 6 showed an IC₅₀ of 101 nM and 27% efficacy. Compounds 32 and 39 displayed respective IC₅₀ of 256 nM and 128 nM, with 27% and 21% efficacy. Compounds 24, 27, and 59 showed respective IC₅₀ of 140 nM, 380 nM and 320 nM, with 24%, 44%, and 27% efficacy.

Tier I ADME properties, including solubility, permeability, and rat liver microsomal (RLM) stability for compounds 4, 6, 24, 27, 32, 39, and 59 of Table 1, above, and niclosamide are provided below, in Table 3.

TABLE 3

| | Solubility | Permeability | RLM stability |
|---|---|---|---|
| Compound | (μg/ml) | (10⁻⁶ cm/sec) | (T_{1/2} min) |
| 6 | 8.46 | 67 | >30 |
| 39 | <1 | 22.3 | >30 |
| 32 | <1 | 5.16 | >30 |
| 4 | 8.4 | 516 | >30 |
| Niclosamide | 1.1 | ND | >30 |
| 59 | <1 | ND | >30 |
| 24 | 6.43 | 127.13 | 22 |
| 27 | 9.24 | 334.4 | >30 |

### Compound Cytotoxicity

Compounds 4, 6, 24, 27, 32, 39, and 59 of Table 2, above, and niclosamide were examined for cytotoxicity.

Compound cytotoxicity was assessed in a BSL-2 counter screen as follows: Cell culture media (MEM, 1% Pen/Strep/GlutaMax, 1% HEPES, 2% HI FBS) was dispensed at 5 μl/well into ARPs and incubated at room temperature to allow for compound dissolution. Vero-E6 (selected for high ACE2 expression) was dispensed into assay plates at 4000 cells/well in 25 μl media. Assay plates were incubated for 72 h at 37 C, 5% CO₂, 90% humidity. Then, 30 μl/well of CellTiter-Glo (Promega #G7573) was dispensed into the assay plate. Luminescence was read using a BMG PHERAstar plate reader following incubation at room temperature for 10 min to measure cell viability. Raw data from each test well was normalized to the average signal of DMSO treated cells (Avg. live; 100% viability) and control compound (hyamine, final assay concentration 100 μM) treated cells (Avg. dead; 0% viability) to calculate % inhibition of CPE using the following formula: % inhibition CPE=100*(Test Cmpd−Avg. Dead)/(Avg. Live−Avg. Dead).

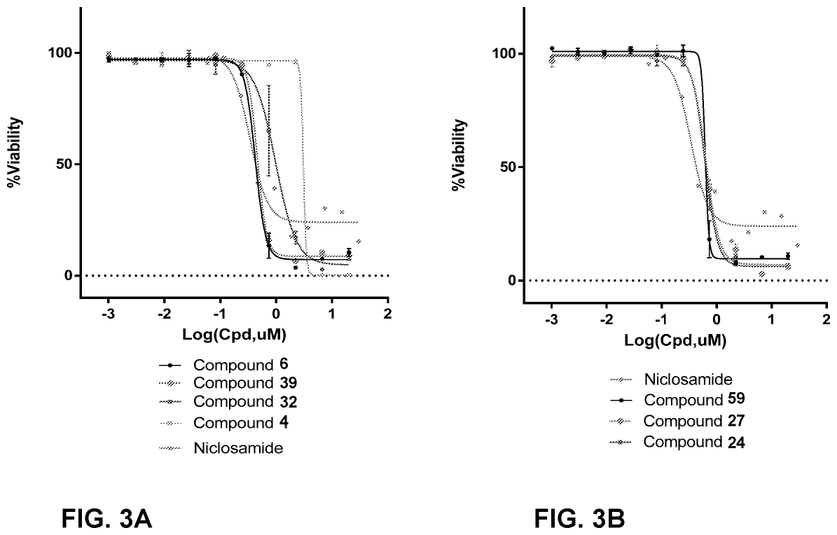

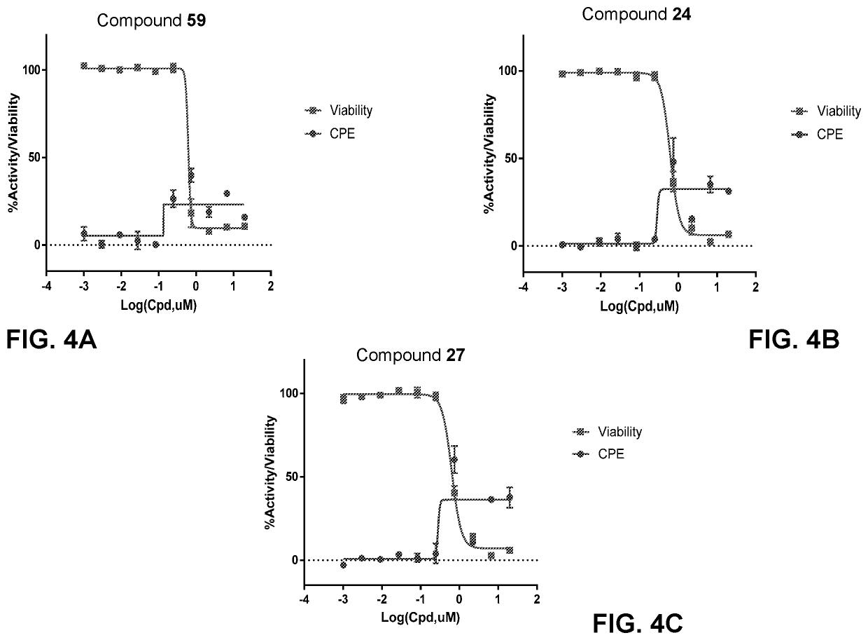

Concentration response curves for compounds 4, 6, 24, 27, 32, 39, and 59 are shown in FIG. 3. Plots comparing the cytopathic effect assay response and the cytotoxicity counter screen response of compounds 24, 27, and 59 are shown in FIG. 4. As shown in FIG. 4, the compounds showed a response comparable to or better than niclosamide, with preferable Tier I ADME properties. The rescue IC₅₀ values obtained in the cytopathic effect assay and the cell viability values obtained in in the cytotoxicity counter assay are shown in Table 4, below.

TABLE 4

| | IC₅₀ (μM) | Efficacy | IC₅₀ (μM) | Efficacy |
|---|---|---|---|---|
| Compound | CPE assay | CPE assay | cell viability | cell viability |
| 39 | 0.12 | 20.6 | 0.45 | −94.6 |
| 32 | 0.32 | 24.9 | 1.01 | −99.6 |
| 4 | 5.1 | −8.3 | 4.05 | −103.9 |
| 6 | 0.1 | 26.5 | 0.4 | −94.8 |
| 59 | 0.14 | 23.7 | 0.45 | −91.7 |
| 24 | 0.32 | 27.9 | 0.57 | −93.1 |
| 27 | 0.4 | 44.31 | 0.64 | −92.9 |

### Example 3

Compounds P1-P9 of Table 1, above, were examined for activity against SARS-CoV-2 using the cytopathic effect assay and cytotoxicity counter screen of Example 2.

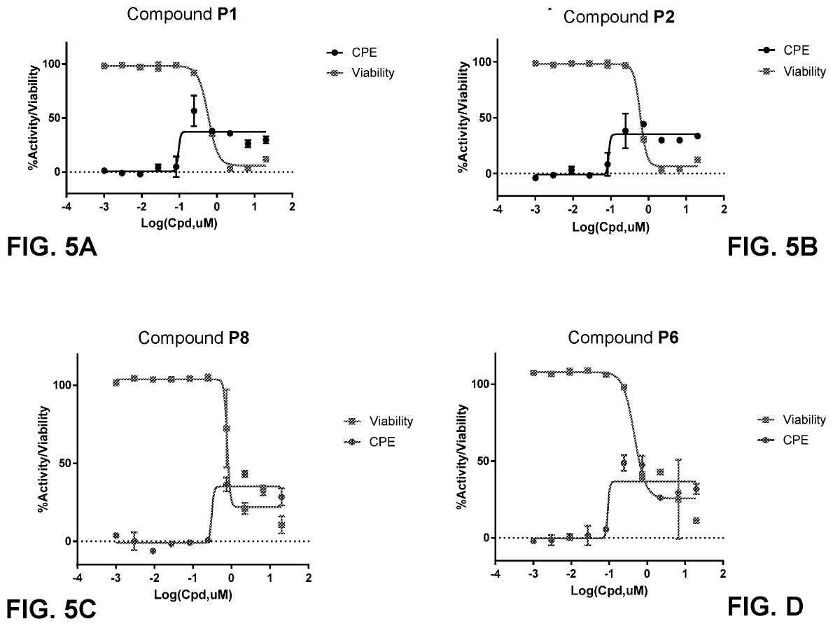

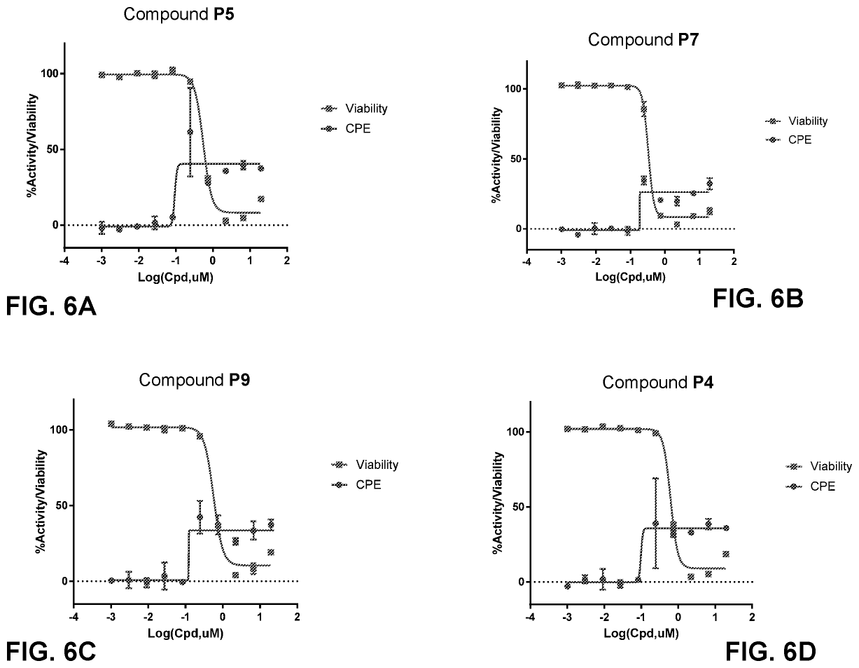

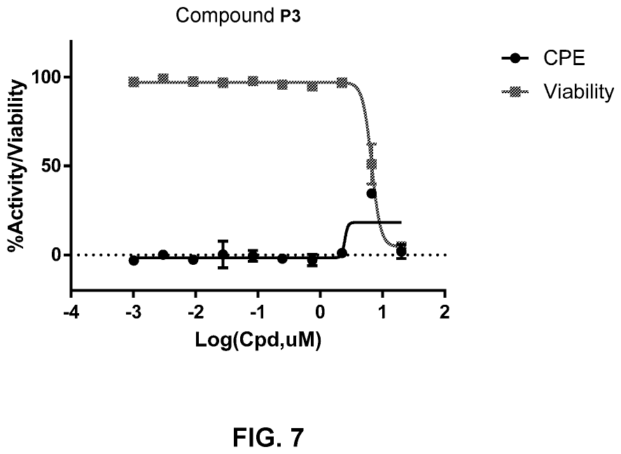

Concentration response rescue curves are shown in FIGS. 5-7. As shown in FIGS. 5-7, the compounds showed a response comparable to or better than niclosamide, with preferable Tier I ADME properties. The rescue IC₅₀ values obtained in the cytopathic effect assay and the cell viability values obtained in in the cytotoxicity counter assay are shown in Table 5, below.

TABLE 5

| | IC₅₀ (μM) | Efficacy | IC₅₀ (μM) | Efficacy |
|---|---|---|---|---|
| Compound | CPE assay | CPE assay | cell viability | cell viability |
| P1 | 0.11 | 34.0 | 0.57 | −96.34 |
| P2 | 0.10 | 36.5 | 0.57 | −95.8 |
| P3 | 4.05 | 40.7 | 7.21 | −99.8 |
| P8 | 0.41 | 36.8 | 0.91 | −90.6 |
| P6 | 0.11 | 37.1 | 0.45 | −82.3 |
| P5 | 0.11 | 37.0 | 0.51 | −91.2 |
| P7 | 0.13 | 26.9 | 0.32 | −94.0 |
| P9 | 0.13 | 35.2 | 0.57 | −91.0 |
| P4 | 0.25 | 36.2 | 0.57 | −93.0 |

Tier I ADME properties, including solubility, permeability, and rat liver microsomal (RLM) stability for compounds P1-P9 of Table 1, above, are provided below, in Table 6.

TABLE 6

| | Solubility | Permeability | RLM stability |
|---|---|---|---|
| Compound | (μg/ml) | (10⁻⁶ cm/sec) | (T_{1/2} min) |
| P1 | <1 | <2.6 | ND |
| P2 | 1.1 | <6.5 | ND |
| P8 | <1 | 982.4 | >30 |
| P6 | <1 | 56.99 (pH5) | ND |
| P5 | <1 | ND | ND |
| P7 | <1 | ND | ND |
| P9 | 4.48 | ND | ND |
| P4 | <1 | 631.8 | ND |
| P3 | >43 | 499.5 (pH5) | >30 |

### Example 4

Compound 6 and niclosamide were examined for activity against SARS-CoV-2 in human bronchial epithelial cells (HBEC; EpiAirway), using a two-step procedure:

In the first part of the experiment, the test compounds were diluted in Assay medium (AIR-100-ASY). The test compound dilutions (0.01, 0.1, 1, 10 micrograms per mL) were added to each insert on the apical layer (0.15 mL) and basal layer (0.85 mL). Following the 1 h treatment, the apical medium was removed, and the basal side was replaced with fresh compound. Virus (0.15 mL) was then added to each insert on the apical layer, removed after 1 h and washed with 0.4 mL TEER buffer. The basal side medium/compound was replaced with 1 mL of Assay medium. Every twenty-four hours, the apical layer of the tissues were washed with 0.4 mL of TEER buffer and aliquoted to separate microfuge tubes. Trizol LS (1.2 mL) was added to each tube, pipetted up and down several times and stored at −80° C. The basal layer supernatant (1 mL) was collected from each well, aliquoted into separate microfuge tubes and stored at −80° C. The basal side medium was replaced with 1 mL of Maintenance Medium.

In the second part of the experiment, RNA was isolated from the trizol samples and the supernatants from all treatments were tittered by plaque assay or TCID50 to determine the amount of virus present in each sample.

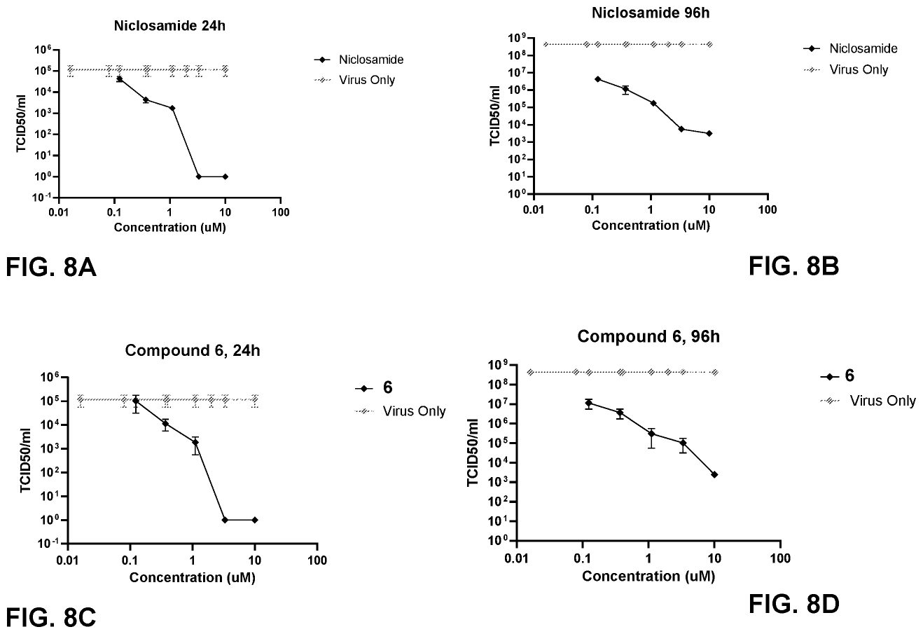

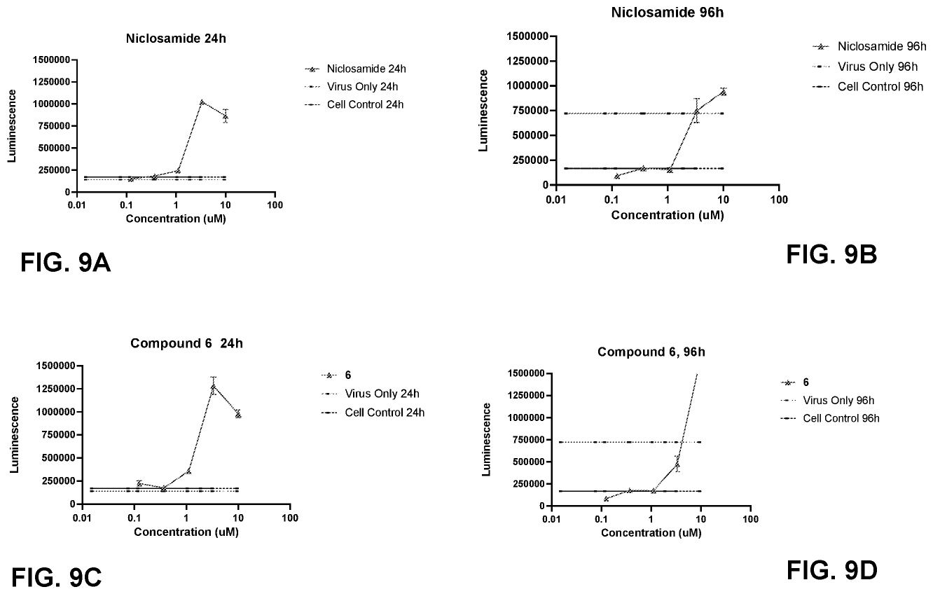

Results are shown in FIGS. 8 and 9. As shown in FIGS. 8 and 9, compound 6 effectively inhibited SARS-CoV-2 in human bronchial epithelial cells at sub-nanomolar and nanomolar concentrations.

### Example 5

The pharmacokinetic (PK) properties of 4-chloro-2-((2-chloro-4-nitrophenyl)carbamoyl)phenyl piperazine-1-carboxylate (compound P3) and niclosamide were studied in male hamsters.

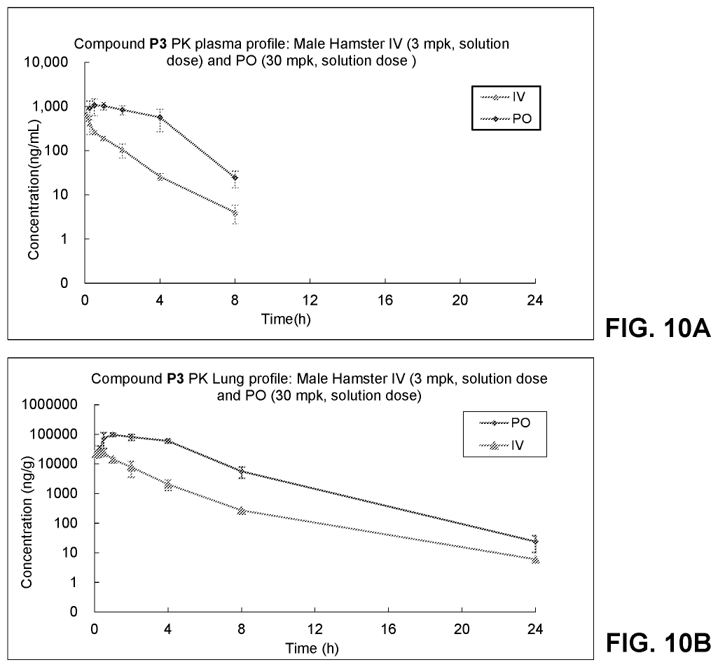

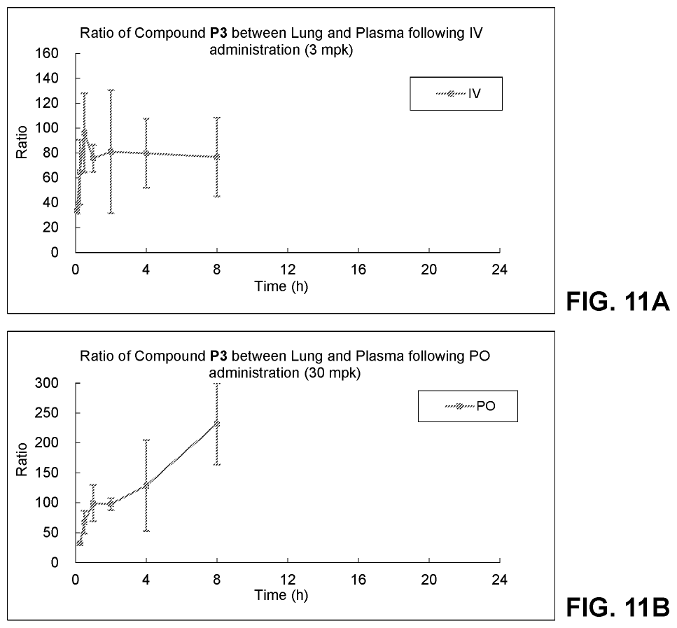

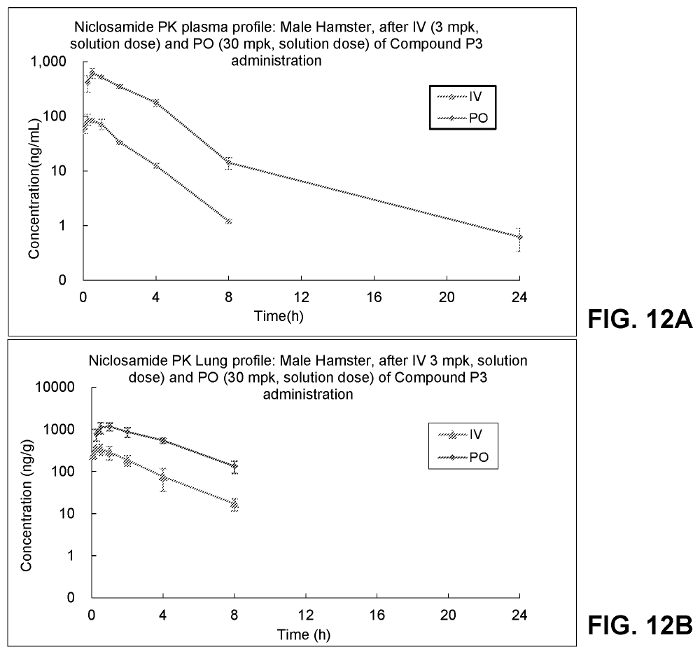

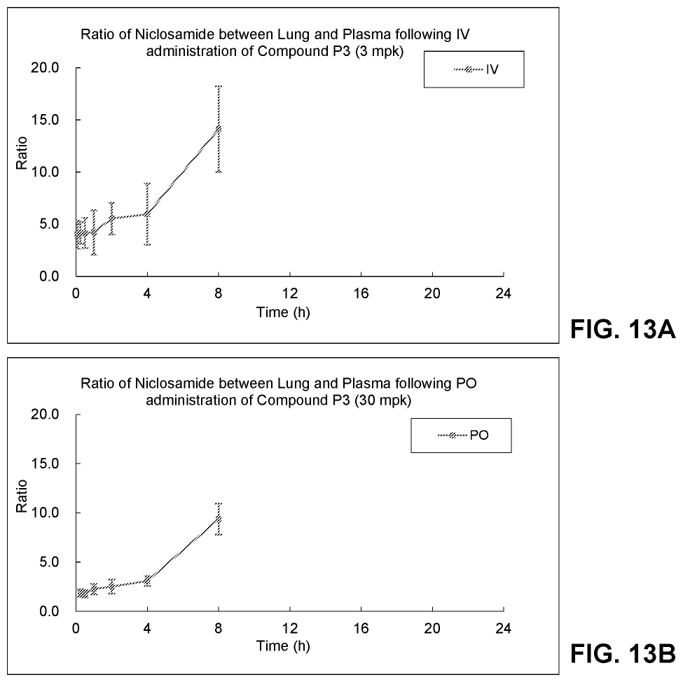

Compound P3 was administered intravenously (3 mg/kg dosing in 20% HP-β-CD in water) or orally (30 mg/kg dosing in 20% HP-β-CD in water). Following administration, the concentration of active metabolite, niclosamide, in plasma and lung tissue samples was monitored over time. Results are shown in Tables 7-18, below, and in FIGS. 10-13.

TABLE 7
Compound P3; plasma concentration following IV administration

| | Conc. (ng/mL) | | | Mean | SD |
|---|---|---|---|---|---|
| Time (h) | Group 1 | Group 2 | Group 3 | (ng/mL) | (ng/mL) |
| 0.083 | 803 | 427 | 745 | 658 | 202 |
| 0.25 | 611 | 282 | 333 | 409 | 177 |
| 0.5 | 247 | 257 | 280 | 261 | 17 |
| 1 | 214 | 184 | 171 | 190 | 22 |
| 2 | 94.5 | 146 | 74.4 | 105 | 37 |
| 4 | 21.0 | 26.1 | 30.3 | 25.8 | 4.7 |
| 8 | 5.97 | 3.45 | 2.53 | 3.98 | 1.78 |
| 24 | BLOQ | BLOQ | BLOQ | NA | NA |

TABLE 8
Compound P3; plasma concentration following PO administration

| | Conc. (ng/mL) | | | Mean | SD |
|---|---|---|---|---|---|
| Time (h) | Group 4 | Group 5 | Group 6 | (ng/mL) | (ng/mL) |
| 0.25 | 450 | 1060 | 1230 | 913 | 410 |
| 0.5 | 1270 | 1360 | 559 | 1063 | 439 |
| 1 | 1080 | 1190 | 812 | 1027 | 194 |
| 2 | 763 | 1060 | 688 | 837 | 197 |
| 4 | 305 | 500 | 891 | 565 | 298 |
| 8 | 35.1 | 15.3 | 22.8 | 24.4 | 10.0 |
| 24 | BLOQ | BLOQ | BLOQ | NA | NA |

TABLE 9
Compound P3; lung concentration following IV administration

| | Conc. (ng/mL) | | | Mean | SD |
|---|---|---|---|---|---|
| Time (h) | Group 1 | Group 2 | Group 3 | (ng/g) | (ng/g) |
| 0.083 | 6840 | 3840 | 5780 | 27360 | 15360 |
| 0.25 | 5360 | 5950 | 6160 | 21440 | 23800 |
| 0.5 | 7760 | 6460 | 4370 | 31040 | 25840 |
| 1 | 3960 | 3020 | 3740 | 15840 | 12080 |
| 2 | 3190 | 1370 | 1310 | 12760 | 5480 |
| 4 | 359 | 726 | 450 | 1436 | 2904 |
| 8 | 67.6 | 65.8 | 68.7 | 270 | 263 |
| 24 | 2.11 | 0.898 | BLOQ | 8.44 | 3.59 |

TABLE 10
Compound P3; lung concentration following PO administration

| | Conc. (ng/mL) | | | Mean | SD |
|---|---|---|---|---|---|
| Time (h) | Group 4 | Group 5 | Group 6 | (ng/g) | (ng/g) |
| 0.25 | 3920 | 8370 | 9330 | 15680 | 33480 |
| 0.5 | 28300 | 18100 | 8350 | 113200 | 72400 |
| 1 | 26500 | 20600 | 26400 | 106000 | 82400 |
| 2 | 16900 | 25500 | 18600 | 67600 | 102000 |
| 4 | 16400 | 12200 | 16300 | 65600 | 48800 |
| 8 | 1700 | 732 | 1770 | 6800 | 2928 |
| 24 | 9.56 | 5.66 | 2.78 | 38.2 | 22.6 |

TABLE 11
Compound P3; lung:plasma concentration ratio following
IV administration

| Time | Animal | Lung | Plasma | Ratio | | |
|---|---|---|---|---|---|---|
| (h) | ID | (ng/g) | (ng/mL) | (Lung/Plasma) | Mean | SD |
| 0.083 | 1 | 27360 | 803 | 34.1 | 33.7 | 2.5 |
| | 2 | 15360 | 427 | 36.0 | | |
| | 3 | 23120 | 745 | 31.0 | | |
| 0.25 | 4 | 21440 | 611 | 35.1 | 64.5 | 26.0 |
| | 5 | 23800 | 282 | 84.4 | | |
| | 6 | 24640 | 333 | 74.0 | | |
| 0.5 | 7 | 31040 | 247 | 126 | 96 | 32 |
| | 8 | 25840 | 257 | 101 | | |
| | 9 | 17480 | 280 | 62.4 | | |
| 1 | 10 | 15840 | 214 | 74.0 | 75.7 | 11.0 |
| | 11 | 12080 | 184 | 65.7 | | |
| | 12 | 14960 | 171 | 87.5 | | |
| 2 | 13 | 12760 | 94.5 | 135 | 81 | 50 |
| | 14 | 5480 | 146 | 37.5 | | |
| | 15 | 5240 | 74.4 | 70.4 | | |
| 4 | 16 | 1436 | 21.0 | 68.4 | 80 | 28 |
| | 17 | 2904 | 26.1 | 111 | | |
| | 18 | 1800 | 30.3 | 59.4 | | |
| 8 | 19 | 270 | 5.97 | 45.3 | 77 | 32 |
| | 20 | 263 | 3.45 | 76.3 | | |
| | 21 | 275 | 2.53 | 109 | | |
| 24 | 22 | 8.44 | BLOQ | NA | NA | NA |
| | 23 | 3.59 | BLOQ | NA | | |
| | 24 | NA | BLOQ | NA | | |

TABLE 12
Compound P3; lung:plasma concentration ratio following PO
administration

| Time | Animal | Lung | Plasma | Ratio | | |
|---|---|---|---|---|---|---|
| (h) | ID | (ng/g) | (ng/ml) | (Lung/Plasma) | Mean | SD |
| 0.25 | 25 | 15680 | 450 | 34.8 | 32.3 | 2.3 |
| | 26 | 33480 | 1060 | 31.6 | | |
| | 27 | 37320 | 1230 | 30.3 | | |
| 0.5 | 28 | 113200 | 1270 | 89.1 | 67.4 | 19.1 |
| | 29 | 72400 | 1360 | 53.2 | | |
| | 30 | 33400 | 559 | 59.7 | | |
| 1 | 31 | 106000 | 1080 | 98.1 | 99 | 30 |
| | 32 | 82400 | 1190 | 69.2 | | |
| | 33 | 105600 | 812 | 130 | | |
| 2 | 34 | 67600 | 763 | 88.6 | 98 | 10 |
| | 35 | 102000 | 1060 | 96.2 | | |
| | 36 | 74400 | 688 | 108 | | |
| 4 | 37 | 65600 | 305 | 215 | 129 | 76 |
| | 38 | 48800 | 500 | 97.6 | | |
| | 39 | 65200 | 891 | 73.2 | | |
| 8 | 40 | 6800 | 35.1 | 194 | 232 | 68 |
| | 41 | 2928 | 15.3 | 191 | | |
| | 42 | 7080 | 22.8 | 311 | | |
| 24 | 43 | 38.2 | BLOQ | NA | NA | NA |
| | 44 | 22.6 | BLOQ | NA | | |
| | 45 | 11.1 | BLOQ | NA | | |

TABLE 13
Niclosamide; plasma concentration following
IV administration of Compound P3

| | Conc. (ng/mL) | | | Mean | SD |
|---|---|---|---|---|---|
| Time (h) | Group 1 | Group 2 | Group 3 | (ng/mL) | (ng/mL) |
| 0.083 | 73.0 | 46.0 | 71.3 | 63.4 | 15.1 |
| 0.25 | 112 | 74.2 | 80.5 | 89 | 20 |
| 0.5 | 81.2 | 77.7 | 92.8 | 83.9 | 7.9 |
| 1 | 84.0 | 79.1 | 54.9 | 72.7 | 15.6 |
| 2 | 33.5 | 33.2 | 34.8 | 33.8 | 0.9 |
| 4 | 11.3 | 13.4 | 13.0 | 12.6 | 1.1 |
| 8 | 1.31 | 1.13 | 1.18 | 1.21 | 0.09 |
| 24 | BLOQ | BLOQ | BLOQ | NA | NA |

TABLE 14
Niclosamide; plasma concentration following
PO administration of Compound P3

| | Conc. (ng/mL) | | | Mean | SD |
|---|---|---|---|---|---|
| Time (h) | Group 4 | Group 5 | Group 6 | (ng/mL) | (ng/mL) |
| 0.25 | 298 | 382 | 568 | 416 | 138 |
| 0.5 | 761 | 620 | 489 | 623 | 136 |
| 1 | 550 | 528 | 483 | 520 | 34 |
| 2 | 352 | 379 | 319 | 350 | 30 |
| 4 | 147 | 196 | 194 | 179 | 28 |
| 8 | 17.0 | 10.4 | 15.2 | 14.2 | 3.4 |
| 24 | 0.447 | 0.462 | 0.943 | 0.617 | 0.282 |

TABLE 15
Niclosamide; lung concentration following
IV administration of Compound P3

| | Conc. (ng/g) | | | Mean | SD |
|---|---|---|---|---|---|
| Time (h) | Group 1 | Group 2 | Group 3 | (ng/g) | (ng/g) |
| 0.083 | 221 | 255 | 243 | 240 | 17 |
| 0.25 | 334 | 353 | 383 | 356 | 25 |
| 0.5 | 420 | 373 | 232 | 342 | 98 |
| 1 | 344 | 171 | 352 | 289 | 102 |
| 2 | 227 | 128 | 208 | 188 | 52 |
| 4 | 57.2 | 124 | 46.4 | 76 | 42 |
| 8 | 20.2 | 10.8 | 20.6 | 17.2 | 5.6 |
| 24 | NA | NA | NA | NA | NA |

TABLE 16
Niclosamide; lung concentration following
PO administration of Compound P3

| | Conc. (ng/g) | | | Mean | SD |
|---|---|---|---|---|---|
| Time (h) | Group 4 | Group 5 | Group 6 | (ng/g) | (ng/g) |
| 0.25 | 492 | 872 | 924 | 763 | 236 |
| 0.5 | 1252 | 1364 | 744 | 1120 | 330 |
| 1 | 1304 | 900 | 1308 | 1171 | 234 |
| 2 | 616 | 988 | 1016 | 873 | 223 |
| 4 | 508 | 496 | 632 | 545 | 75 |
| 8 | 182 | 102 | 116 | 133 | 43 |
| 24 | NA | NA | NA | NA | NA |

TABLE 17
Niclosamide; lung:plasma concentration ratio following
IV administration of Compound P3

| Time | Animal | Lung | Plasma | Ratio | | |
|---|---|---|---|---|---|---|
| (h) | ID | (ng/g) | (ng/ml) | (Lung/Plasma) | Mean | SD |
| 0.083 | 1 | 221 | 73.0 | 3.03 | 3.99 | 1.35 |
| | 2 | 255 | 46.0 | 5.54 | | |
| | 3 | 243 | 71.3 | 3.41 | | |
| 0.25 | 4 | 334 | 112 | 2.98 | 4.16 | 1.03 |
| | 5 | 353 | 74.2 | 4.75 | | |
| | 6 | 383 | 80.5 | 4.76 | | |
| 0.5 | 7 | 420 | 81.2 | 5.17 | 4.16 | 1.44 |
| | 8 | 373 | 77.7 | 4.80 | | |
| | 9 | 232 | 92.8 | 2.50 | | |
| 1 | 10 | 344 | 84.0 | 4.09 | 4.22 | 2.13 |
| | 11 | 171 | 79.1 | 2.16 | | |
| | 12 | 352 | 54.9 | 6.41 | | |
| 2 | 13 | 227 | 33.5 | 6.78 | 5.54 | 1.51 |
| | 14 | 128 | 33.2 | 3.87 | | |
| | 15 | 208 | 34.8 | 5.98 | | |
| 4 | 16 | 57.2 | 11.3 | 5.06 | 5.95 | 2.93 |
| | 17 | 124 | 13.4 | 9.22 | | |
| | 18 | 46.4 | 13.0 | 3.57 | | |
| 8 | 19 | 20.2 | 1.31 | 15.4 | 14.1 | 4.1 |
| | 20 | 10.8 | 1.13 | 9.52 | | |
| | 21 | 20.6 | 1.18 | 17.4 | | |
| 24 | 22 | NA | BLOQ | NA | NA | NA |
| | 23 | NA | BLOQ | NA | | |
| | 24 | NA | BLOQ | NA | | |

TABLE 18
Niclosamide; lung:plasma concentration ratio following PO
administration of Compound P3

| Time | Animal | Lung | Plasma | Ratio | | |
|---|---|---|---|---|---|---|
| (h) | ID | (ng/g) | (ng/ml) | (Lung/Plasma) | Mean | SD |
| 0.25 | 25 | 492 | 298 | 1.65 | 1.85 | 0.37 |
| | 26 | 872 | 382 | 2.28 | | |
| | 27 | 924 | 568 | 1.63 | | |
| 0.5 | 28 | 1252 | 761 | 1.65 | 1.79 | 0.36 |
| | 29 | 1364 | 620 | 2.20 | | |
| | 30 | 744 | 489 | 1.52 | | |
| 1 | 31 | 1304 | 550 | 2.37 | 2.26 | 0.51 |
| | 32 | 900 | 528 | 1.70 | | |
| | 33 | 1308 | 483 | 2.71 | | |
| 2 | 34 | 616 | 352 | 1.75 | 2.51 | 0.72 |
| | 35 | 988 | 379 | 2.61 | | |
| | 36 | 1016 | 319 | 3.18 | | |
| 4 | 37 | 508 | 147 | 3.46 | 3.08 | 0.49 |
| | 38 | 496 | 196 | 2.53 | | |
| | 39 | 632 | 194 | 3.26 | | |
| 8 | 40 | 182 | 17.0 | 10.7 | 9.4 | 1.6 |
| | 41 | 102 | 10.4 | 9.77 | | |
| | 42 | 116 | 15.2 | 7.63 | | |
| 24 | 43 | NA | 0.447 | NA | NA | NA |
| | 44 | NA | 0.462 | NA | | |
| | 45 | NA | 0.943 | NA | | |

Notably, whether administered intravenously or orally, compound P3 provided a higher overall concentration of active metabolite, and moreover provided a significantly higher amount of active metabolite in lung tissue (e.g., relative to plasma concentration).

### Example 6

The pharmacokinetic (PK) properties of 4-chloro-2-((2-chloro-4-nitrophenyl)carbamoyl)phenyl piperazine-1-carboxylate (compound P3) were studied in mice.

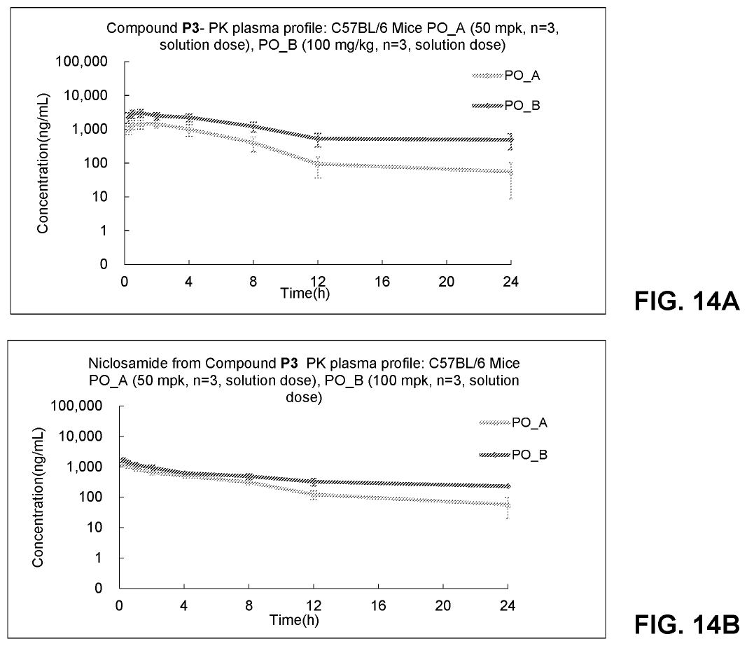

Compound P3 was dosed orally at 50 and 100 mg/kg (in 10% DMA, 90% PEG 400) at a dosing concentration of 5 and 10 mg/ml respectively. Following administration, the concentration of P3 and the metabolite niclosamide in plasma samples were monitored over time. Results are shown in Tables 19-20, below, and in FIG. 14.

TABLE 19
Compound P3 PO Pharmacokinetic Plasma Profile in C57BL/6 Mice n = 3

| | | Dosing | | | | | | | | AUC_% | | | | |
|---|---|---|---|---|---|---|---|---|---|---|---|---|---|---|
| | | Level | | Cl_obs | t_{1/2} | tₘₐₓ | Cₘₐₓ | AUCₗₐₛₜ | AUC_{Inf} | Extrap | MRT | AUCₗₐₛₜ/D | Vₛₛ_obs | F |
| Animal | Route | (mg/kg) | Formulation | (mL/min/kg) | (h) | (h) | (ng/ml) | (h*ng/ml) | (h*ng/ml) | (%) | (h) | (h*mg/mL) | (L/kg) | (%) |
| Mouse 1 | PO_A | 50 | 10% DMA, | NA | NA | 0.500 | 1140 | 6357 | 242800 | 97.4 | NA | 127 | NA | NA |
| Mouse 2 | | | 90% | NA | 4.58 | 1.00 | 1920 | 12332 | 12585 | 2.01 | NA | 247 | NA | NA |
| Mouse 3 | | | PEG400 | NA | 3.70 | 2.00 | 1560 | 10489 | 10595 | 1.00 | NA | 210 | NA | NA |
| Mean | | | | NA | 4.14 | 2.00 | 1457 | 9726 | 88660 | 33.5 | NA | 195 | NA | NA |
| SD | | | | NA | NA | NA | NA | 3060 | 133493 | 55.4 | NA | 61 | NA | NA |
| Mouse 4 | PO_B | 100 | 10% DMA, | NA | 12.4 | 1.00 | 2970 | 25975 | 33619 | 22.7 | NA | 260 | NA | NA |
| Mouse 5 | | | 90% | NA | 13.8 | 1.00 | 2340 | 18757 | 24577 | 23.7 | NA | 188 | NA | NA |
| Mouse 6 | | | PEG400 | NA | 19.8 | 1.00 | 3880 | 34617 | 56087 | 38.3 | NA | 346 | NA | NA |
| Mean | | | | NA | 15.3 | 1.00 | 3063 | 26450 | 38094 | 28.2 | NA | 264 | NA | NA |
| SD | | | | NA | 3.9 | NA | NA | 7941 | 16225 | 8.7 | NA | 79 | NA | NA |

TABLE 20
Niclosamide from Compound P3 PO Pharmacokinetic Plasma Profile in C57BL/6 Mice n = 3

| | | Dosing | | | | | | | | AUC_% | | | | |
|---|---|---|---|---|---|---|---|---|---|---|---|---|---|---|
| | | Level | | Cl_obs | t_{1/2} | tₘₐₓ | Cₘₐₓ | AUCₗₐₛₜ | AUC_{Inf} | Extrap | MRT | AUCₗₐₛₜ/D | Vₛₛ_obs | F |
| Animal | Route | (mg/kg) | Formulation | (mL/min/kg) | (h) | (h) | (ng/mL) | (h*ng/mL) | (h*ng/ml) | (%) | (h) | (h*mg/mL) | (L/kg) | (%) |
| Mouse 1 | PO_A | 37.2 | NA | NA | 15.7 | 0.25 | 1320 | 6041 | 8233 | 26.6 | NA | 162 | NA | NA |
| Mouse 2 | | | | NA | 6.58 | 0.25 | 1300 | 6515 | 7012 | 7.09 | NA | 175 | NA | NA |
| Mouse 3 | | | | NA | 4.12 | 0.25 | 1120 | 6603 | 6734 | 1.95 | NA | 177 | NA | NA |
| Mean | | | | NA | 8.8 | 0.25 | 1247 | 6386 | 7327 | 11.9 | NA | 172 | NA | NA |
| SD | | | | NA | 6.1 | NA | NA | 303 | 797 | 13.0 | NA | 8 | NA | NA |
| Mouse 4 | PO_B | 74.4 | NA | NA | 11.0 | 0.25 | 1890 | 12590 | 15931 | 21.0 | NA | 169 | NA | NA |
| Mouse 5 | | | | NA | 18.9 | 0.25 | 1680 | 10909 | 17433 | 37.4 | NA | 147 | NA | NA |
| Mouse 6 | | | | NA | 27.0 | 0.25 | 1410 | 9491 | 18826 | 49.6 | NA | 128 | NA | NA |
| Mean | | | | NA | 19.0 | 0.25 | 1660 | 10996 | 17397 | 36.0 | NA | 148 | NA | NA |
| SD | | | | NA | 8.0 | NA | NA | 1551 | 1448 | 14.4 | NA | 21 | NA | NA |

Compound P3 was effectively converted in vivo to the active niclosamide. A Cₘₐₓ of 3.8 μM was achieved with the 50 mg/kg dose while a Cₘₐₓ of 5 μM was achieved with the 100 mg/kg dose. The half-lives were 8.8 and 19 h for the 50 and 100 mg/kg doses respectively. Notably, oral administration of Compound P3 resulted in much improved plasma exposures of niclosamide than that reported for oral niclosamide administration.

### Example 7

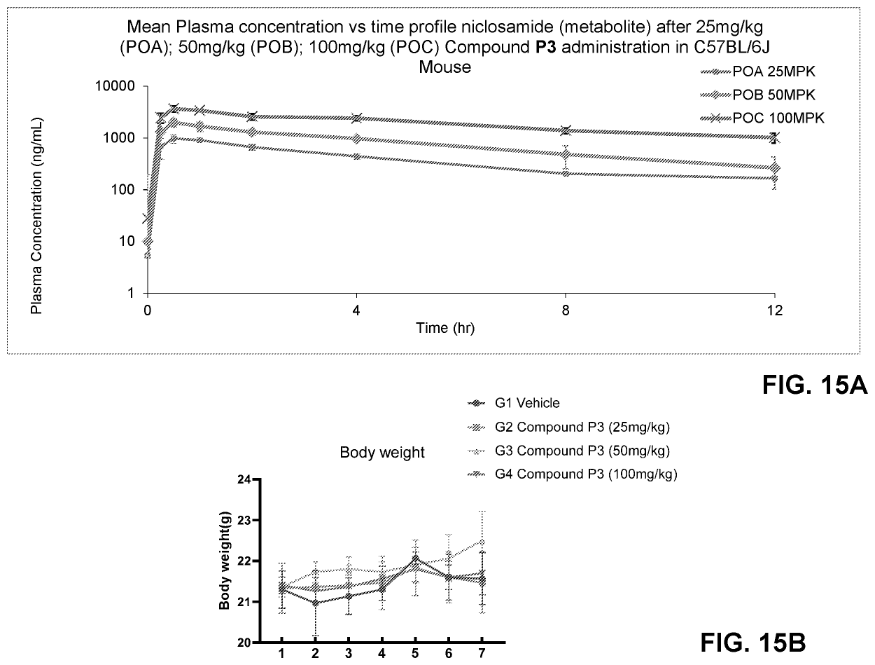

Tolerability studies of 4-chloro-2-((2-chloro-4-nitrophenyl)carbamoyl)phenyl piperazine-1-carboxylate (compound P3) were conducted in mice. Compound P3 was dosed orally at 25, 50 and 100 mg/kg (in 5% EtOH, 60% PEG-300 and 35% (20% HP-β-CD in water)) for 7 days, QD doing in C57BL/6J mice. PK was performed on the 7th day and the plasma concentrations of Compound P3 and the active metabolite niclosamide were measured for 12 h post dosing. Body weights were monitored throughout the study. Results are show in tables 21-23 below, and in FIG. 15.

TABLE 21
Summary of Niclosamide (when Compound P3 was dosed at 25 mg/kg)
Pharmacokinetic Parameters

| PK parameters | Unit | Mouse 16 | Mouse 18 | Mouse 19 | Mean | SD | CV(%) |
|---|---|---|---|---|---|---|---|
| T_{1/2} | h | 6.10 | 4.49 | 4.22 | 4.94 | 1.02 | 20.7 |
| Tₘₐₓ | h | 0.500 | 0.500 | 2.00 | 1.00 | 0.87 | 86.6 |
| Cₘₐₓ | ng/ml | 1120 | 1170 | 703 | 998 | 256 | 25.7 |
| AUCₗₐₛₜ | h*ng/ml | 4606 | 4974 | 4473 | 4684 | 260 | 5.54 |
| AUC_{Inf} | h*ng/ml | 6429 | 6055 | 5221 | 5902 | 619 | 10.5 |
| AUC_% Extrap_obs | % | 28.4 | 17.9 | 14.3 | 20.2 | 7.3 | 36.1 |
| MRT_{Inf}_obs | h | 8.74 | 6.39 | 6.26 | 7.13 | 1.39 | 19.6 |
| AUClast/D | h*mg/mL | 248 | 267 | 240 | 252 | 14 | 5.54 |

TABLE 22
Summary of Niclosamide (when Compound P3 was dosed at 50 mg/kg)
Pharmacokinetic Parameters

| PK parameters | Unit | Mouse 5 | Mouse 8 | Mouse 26 | Mean | SD | CV(%) |
|---|---|---|---|---|---|---|---|
| T_{1/2} | h | 5.85 | 4.99 | 2.30 | 4.38 | 1.85 | 42.2 |
| Tₘₐₓ | h | 0.500 | 0.500 | 1.00 | 0.67 | 0.29 | 43.3 |
| Cₘₐₓ | ng/ml | 1820 | 2410 | 2020 | 2083 | 300 | 14.4 |
| AUCₗₐₛₜ | h*ng/ml | 10625 | 9048 | 9320 | 9664 | 843 | 8.72 |
| AUC_{Inf} | h*ng/ml | 13991 | 11282 | 9602 | 11625 | 2214 | 19.0 |
| AUC_% Extrap_obs | % | 24.1 | 19.8 | 2.94 | 15.6 | 11.2 | 71.6 |
| MRT_{Inf}_obs | h | 8.52 | 6.82 | 3.84 | 6.39 | 2.37 | 37.1 |
| AUClast/D | h*mg/mL | 286 | 243 | 250 | 260 | 23 | 8.72 |

TABLE 23
Summary of Niclosamide (when Compound P3 was dosed at 100 mg/kg)
Pharmacokinetic Parameters

| PK parameters | Unit | Mouse 3 | Mouse 10 | Mouse 20 | Mean | SD | CV(%) |
|---|---|---|---|---|---|---|---|
| T_{1/2} | h | 6.90 | 7.13 | 5.27 | 6.43 | 1.02 | 15.8 |
| Tₘₐₓ | h | 0.500 | 1.00 | 0.500 | 0.67 | 0.29 | 43.3 |
| Cₘₐₓ | ng/ml | 3640 | 3230 | 4180 | 3683 | 476 | 12.9 |
| AUCₗₐₛₜ | h*ng/ml | 22810 | 24000 | 22889 | 23233 | 665 | 2.86 |
| AUC_{Inf} | h*ng/ml | 32492 | 36862 | 29119 | 32824 | 3882 | 11.8 |
| AUC_% Extrap_obs | % | 29.8 | 34.9 | 21.4 | 28.7 | 6.8 | 23.7 |
| MRT_{Inf}_obs | h | 9.91 | 11.0 | 7.57 | 9.5 | 1.8 | 18.5 |
| AUClast/D | h*mg/mL | 307 | 322 | 308 | 312 | 9 | 2.86 |

All three doses were tolerable for the study period, with no gross findings from the study. There was no dose dependent weight loss observed in the animals. A dose dependent increase in the plasma concentrations of niclosamide was seen in the three doses.

### Example 8

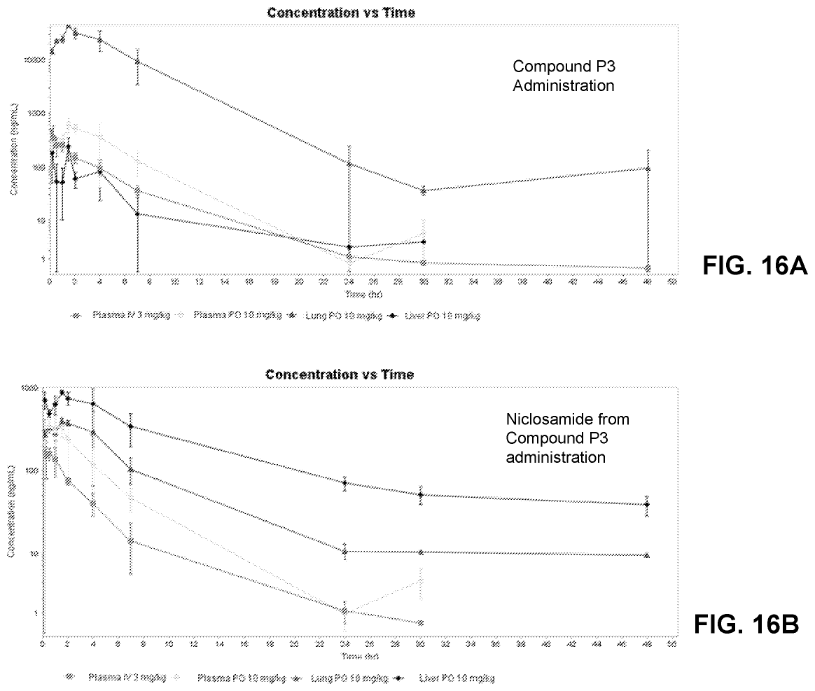

The pharmacokinetic (PK) properties of 4-chloro-2-((2-chloro-4-nitrophenyl)carbamoyl)phenyl piperazine-1-carboxylate (compound P3) were studied in mice. Compound P3 was dosed intravenously and orally at 3 and 10 mg/kg respectively (in 5% EtOH, 60% PEG-300 and 35% (20% HP-b-CD in water)). Following administration, the concentration of compound P3 and the active metabolite niclosamide in plasma, lung and liver samples were monitored over time. Results are shown in tables 24-25 and in FIG. 16.

TABLE 24

| | | Compound P3 | | | Niclosamide | | |
|---|---|---|---|---|---|---|---|
| | | Sample | | | | | |
| | | Plasma | Lung | Liver | Plasma | Lung | Liver |
| | | Route | | | | | |
| Parameter | Units | PO | PO | PO | PO | PO | PO |
| Dose | mg/kg | 10 | 10 | 10 | | | |
| AUC0-last | hr*ng/ml | 3510 | 244000 | 651 | 1600 | 3060 | 8850 |
| SE_AUClast | hr*ng/ml | 594 | 37400 | 131 | 182 | 392 | 997 |
| AUC0-INF | hr*ng/mL | 3530 | 244000 | 657 | 1620 | 3080 | 9250 |
| AUC0-INF/D | hr*kg*ng/ml/mg | 353 | 24400 | 65.7 | | | |
| AUC_% Extrap | % | 0.5 | 0.2 | 0.9 | 1.0 | 0.6 | 4.4 |
| t1/2 | hr | 2.2 | 2.8 | 4.0 | 2.7 | 4.3 | 7.3 |
| Tmax | hr | 1.5 | 1.5 | 1.5 | 0.5 | 1.5 | 1.5 |
| Cmax | ng/mL | 606 | 44200 | 240 | 346 | 397 | 882 |
| Cmax/D | kg*ng/ml/mg | 60.6 | 4420 | 24 | | | |
| AUC ratio | | | 69 | 0.19 | | 1.9 | 5.7 |
| (tissue/plasma) | | | | | | | |
| AUC ratio | | | | | 0.46 | 0.013 | 14 |
| (niclosamide/ | | | | | | | |
| Compound P3) | | | | | | | |

TABLE 25

| | | Compound | Compound | | |
|---|---|---|---|---|---|
| Parameter | Units | P3 | P3 | Niclosamide | Niclosamide |
| Species | | mouse | mouse | mouse | mouse |
| Sample | | Plasma | Plasma | Plasma | Plasma |
| Route | | IV | PO | IV | PO |
| Dose | mg/kg | 3 | 10 | | |
| Tlast | hr | 48 | 30 | 30 | 30 |
| Clast | ng/mL | 0.163 | 5.23 | 0.329 | 4.04 |
| AUClast | hr*ng/mL | 1280 | 3510 | 585 | 1600 |
| AUCLast/D | hr*kg*ng/mL/mg | 425 | 351 | | |
| SE_AUClast | hr*ng/mL | 83.7 | 594 | 64.4 | 182 |
| AUCINF_obs | hr*ng/mL | 1280 | 3530 | 587 | 1620 |
| AUCINF/D | hr*kg*ng/mL/mg | 426 | 353 | | |
| AUC_%Extrap_obs | % | 0.06 | 0.5 | 0.3 | 1.0 |
| t½ | hr | 3.3 | 2.2 | 3.6 | 2.7 |
| Tmax | hr | 0.083 | 1.5 | 0.083 | 0.5 |
| Cmax | ng/mL | 461 | 606 | 168 | 346 |
| Cmax/D | kg*ng/mL/mg | 154 | 60.6 | | |
| C0 | ng/mL | 536 | | | |
| Cl | mL/min/kg | 39 | | | |
| Vss | L/kg | 8.9 | | | |
| F | % | | 83 | | |
| AUC ratio | | | | | |
| (tissue/plasma) | | | | | |
| AUC ratio | | | | 0.46 | 0.46 |
| (niclosamide/ | | | | | |
| Compound P3) | | | | | |

An 83% bioavailability was observed for Compound P3 with moderate clearance. The exposure ratio between active drug/prodrug was approx. 0.46 for both routes. The active drug niclosamide was 2-fold higher in lungs than plasma through PO dosing route.

### Example 9

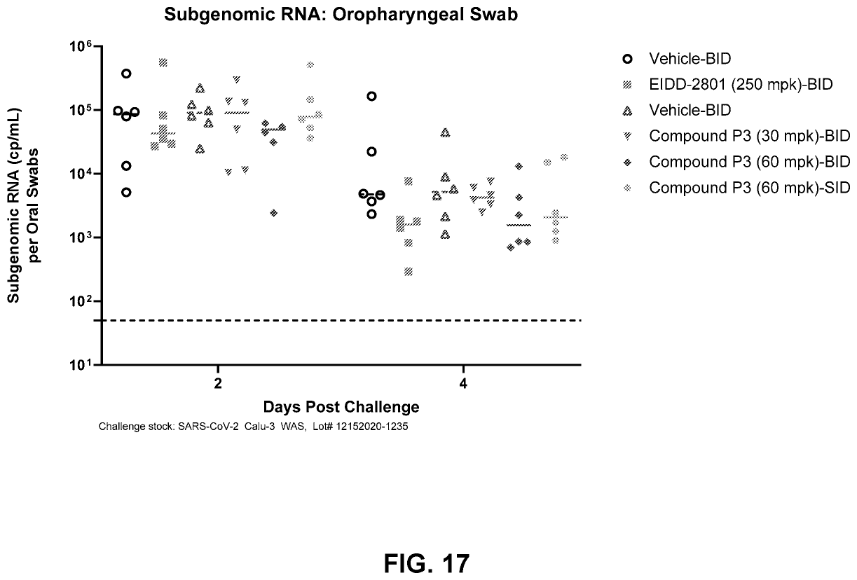

Efficacy of Compound P3 was evaluated in a Golden Syrian Hamster Efficacy Model of SARS-CoV-2. Hamsters were divided into 6 groups, with 6 animals per group. EIDD-2801 (molnupiravir) was chosen as the positive control at 250 mpk BID oral dose. Two vehicle groups, EIDD dosing vehicle and the compound P3 dosing vehicle were used as groups 1 and 3. Compound P3 was dosed at 30 mpk BID, 60 mpk BID and 60 mpk SID in groups 4, 5 and 6 respectively. Treatment was started one day before the viral challenge and was continued for 3 more days. Oropharyngeal swabs were collected on days 2 and 4 post challenge and max bleed was performed on day 4 following which tissue samples were collected for TCID₅₀ determination and histopathological and IHC findings. Results are shown in FIG. 17.

Genomic and subgenomic SARS-CoV-2 RNA were quantified in the oropharyngeal swabs on days 2 and 4 post dosing. A dose dependent decrease in the subgenomic RNA was seen and was comparable to the EIDD group. IHC immunoreactivity staining in the right lung also appeared lowered in severity in the EIDD group and 60 mpk BID Compound P3 group.

It is understood that the examples and embodiments described herein are for illustrative purposes only and that various modifications or changes in light thereof will be suggested to persons skilled in the art and are to be incorporated within the spirit and purview of this application and scope of the appended claims. All publications, patents, and patent applications cited herein are hereby incorporated herein by reference for all purposes.

1. A compound of Formula I 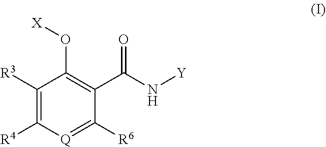
or a pharmaceutically acceptable salt thereof, wherein
X is —PO(R^{1A})(R^{2A}); and
R^{1A} and R^{2A} are each independently C₁-C₆ alkoxy;
or X is —C(O)R^{1B}; and
R^{1B} is C₁-C₁₂ alkyl, C₁-C₁₂ haloalkyl, hydroxy(C₁-C₈ alkyl), alkoxy(C₁-C₈ alkyl), or amino(C₁-C₈ alkyl), cycloalkyl optionally substituted with one or more R¹⁴, aryl optionally substituted with one or more R¹⁴, heteroaryl optionally substituted with one or more R¹⁴, or heterocycloalkyl optionally substituted with one or more R¹⁴;
or X is —C(O)NR^{1C}R^{2C}; and
R^{1C} and R^{2C} are each independently hydrogen, C₁-C₆ alkyl, C₁-C₆ haloalkyl, hydroxy(C₁-C₆ alkyl), alkoxy(C₁-C₆ alkyl), or amino(C₁-C₆ alkyl), or
R^{1C} and R^{2C}, together with the atoms to which they are attached, form a 5- to 8-membered monocyclic heterocyclyl optionally substituted with one or more R¹⁵ selected from C₁-C₆ alkyl, —NH₂, —NH(C₁-C₆ alkyl), —N(C₁-C₆ alkyl)₂, —OH, C₁-C₆ alkoxy, cycloalkyl, or heterocycloalkyl;
or R^{1C} and R^{2C}, together with the atoms to which they are attached, form a bicyclic heterocyclyl selected from a 5- to 8-membered monocyclic heterocyclyl bridged with 1-3 additional carbon atoms, and a 5- to 8-membered monocyclic heterocyclyl fused to a monocyclic cycloalkyl, monocyclic aryl, a monocyclic heterocycloalkyl, or a monocyclic heteroaryl;
R³ and R⁴ are each independently hydrogen, halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, C₁-C₆ haloalkoxy, —SH, —CONH₂, —CONH(C₁-C₆ alkyl), —CON(C₁-C₆ alkyl)₂, —CO₂H, —CO₂(C₁-C₆ alkyl), —CHO, —CO(C₁-C₆ alkyl), or —S(O)₀₋₂(C₁-C₆ alkyl), or
R³ and R⁴, together with the atoms to which they are attached, form a 5- or 6-membered aryl, heteroaryl, or heterocyclyl optionally substituted with one or more R¹⁴;
Q is N;
or Q is CR⁵; and
R⁵ is hydrogen, halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, C₁-C₆ haloalkoxy, —SH, —CONH₂, —CONH(C₁-C₆ alkyl), —CON(C₁-C₆ alkyl)₂, —CO₂H, —CO₂(C₁-C₆ alkyl), —CHO, —CO(C₁-C₆ alkyl), —S(O)₀₋₂(C₁-C₆ alkyl), cycloalkyl optionally substituted with one or more R¹⁴, aryl optionally substituted with one or more R¹⁴, heteroaryl optionally substituted with one or more R¹⁴, or heterocycloalkyl optionally substituted with one or more R¹⁴;
R⁶ is hydrogen, halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, C₁-C₆ haloalkoxy, —SH, —CONH₂, —CONH(C₁-C₆ alkyl), —CON(C₁-C₆ alkyl)₂, —CO₂H, —CO₂(C₁-C₆ alkyl), —CHO, —CO(C₁-C₆ alkyl), —S(O)₀₋₂(C₁-C₆ alkyl), cycloalkyl optionally substituted with one or more R¹⁴, aryl optionally substituted with one or more R¹⁴, heteroaryl optionally substituted with one or more R¹⁴, or heterocycloalkyl optionally substituted with one or more R¹⁴, or
R⁵ and R⁶, together with the atoms to which they are attached, form a 5- or 6-membered aryl, heteroaryl, or heterocyclyl optionally substituted with one or more R¹⁴;
Y is 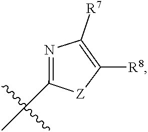
wherein
Z is O or S;
R⁷ and R⁸ are each independently hydrogen, halogen, —NO₂, —CN, C₁-C₆ alkyl, or C₁-C₆ haloalkyl;
or Y is 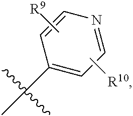
wherein
R⁹ and R¹⁰ are each independently hydrogen, halogen, —NO₂, —CN, C₁-C₆ alkyl, or C₁-C₆ haloalkyl;
or Y is 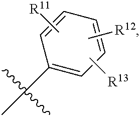
wherein
R¹¹, R¹², and R¹³ are each independently hydrogen, halogen, —NO₂, —CN, C₁-C₆ alkyl, C₁-C₆ haloalkyl, —NH₂, —NH(C₁-C₆ alkyl), —N(C₁-C₆ alkyl)₂, —NHCO(C₁-C₆ alkyl), —N(C₁-C₆ alkyl)CO(C₁-C₆ alkyl), —OH, C₁-C₆ alkoxy, C₁-C₆ haloalkoxy, —SH, —CONH₂, —CONH(C₁-C₆ alkyl), —CON(C₁-C₆ alkyl)₂, —CO₂H, —CO₂(C₁-C₆ alkyl), —CHO, —CO(C₁-C₆ alkyl), —S(O)₀₋₂(C₁-C₆ alkyl), —S(O)₀₋₂NH₂, —S(O)₀₋₂NH(C₁-C₆ alkyl), or —S(O)₀₋₂N(C₁-C₆ alkyl)₂, or
R¹¹ and R¹³, together with the atoms to which they are attached, form a 5- or 6-membered aryl, heteroaryl, or heterocyclyl optionally substituted with one or more R¹⁴; and
each R¹⁴ is independently halogen, —NO₂, —CN, —NH₂, —NH(C₁-C₄ alkyl), —N(C₁-C₄ alkyl)₂, —OH, C₁-C₄ alkyl, C₁-C₄ haloalkyl, C₁-C₄ alkoxy, or C₁-C₄ haloalkoxy;
wherein the compound of Formula I is not
4-chloro-2-((2-chloro-4-nitrophenyl)carbamoyl)phenyl octanoate;
4-chloro-2-((2-chloro-4-nitrophenyl)carbamoyl)phenyl acetate;
4-chloro-2-((2-chloro-4-nitrophenyl)carbamoyl)phenyl benzoate;
4-chloro-2-((2-chloro-4-nitrophenyl)carbamoyl)phenyl diethyl phosphate; or
4-chloro-2-((2-chloro-4-nitrophenyl)carbamoyl)phenyl 4-methylpiperazine-1-carboxylate.

2. The compound of claim 1, wherein X is —PO(R^{1A})(R^{2A}), and R^{1A} and R^{2A} are each independently C₁-C₆ alkoxy.

3. The compound of claim 1, wherein X is —C(O)R^{1B}, and R^{1B} is aryl optionally substituted with one or more R¹⁴ independently selected from halogen and C₁-C₄ haloalkyl.

4. The compound of claim 1, wherein X is —C(O)R^{1B}, and R^{1B} is C₁-C₁₂ alkyl.

5. The compound of claim 1, wherein X is —C(O)NR^{1C}R^{2C}, and R^{1C} and R^{2C} are each independently hydrogen, C₁-C₆ alkyl, or amino(C₁-C₆ alkyl), or R^{1C} and R^{2C}, together with the atoms to which they are attached, form a 5- to 8-membered heterocyclyl or a bicyclic heterocyclyl.

6. The compound of claim 1, wherein X is —C(O)NR^{1C}R^{2C}, and R^{1C} and R^{2C}, together with the atoms to which they are attached, form a bicyclic heterocyclyl selected from
a 5- to 8-membered monocyclic heterocyclyl bridged with 1-3 additional carbon atoms, and
a 5- to 8-membered monocyclic heterocyclyl fused to a monocyclic cycloalkyl, monocyclic aryl, a monocyclic heterocycloalkyl, or a monocyclic heteroaryl.

7. The compound of claim 1, wherein X is —C(O)NR^{1C}R^{2C}, and R^{1C} and R^{2C}, together with the atoms to which they are attached, form a 5- to 6-membered monocyclic heterocyclyl optionally substituted with one R¹⁵ is selected from C₁-C₆ alkyl (e.g., —CH₃), C₁-C₆ alkoxy (e.g., —OCH₃), —OH, and —NH₂.

8. The compound of claim 1, wherein X is —C(O)NR^{1C}R^{2C}, and R^{1C} and R^{2C}, together with the atoms to which they are attached, form a 5- or 6-membered monocyclic heterocyclyl.

9. The compound of claim 1, wherein the compound is of Formula I-A

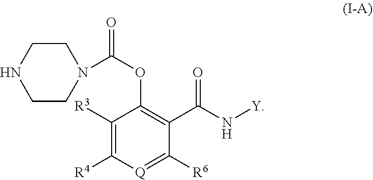

10. The compound of any of claims 1-9, wherein Q is CR⁵.

11. The compound of any of claims 1-10, wherein
R³ and R⁴ are each independently hydrogen, halogen, or C₁-C₆ alkoxy, or
R³ and R⁴, together with the atoms to which they are attached, form a 6-membered aryl optionally substituted with one or more R¹⁴ independently selected from halogen, —NO₂, and —CN.

12. The compound of any of claims 1-10, wherein R³ and R⁴ are each independently hydrogen, halogen, or C₁-C₆ alkoxy.

13. The compound of any of claims 1-10, wherein R³ and R⁴ are each independently hydrogen or halogen.

14. The compound of any of claims 1-10, wherein R³ is hydrogen and R⁴ is halogen or C₁-C₆ alkoxy.

15. The compound of any of claims 1-10, wherein R³ and R⁴ are each hydrogen.

16. The compound of any of claims 1-15, wherein
Q is CR⁵, and R⁵ and R⁶ are each independently hydrogen, halogen, —NO₂, C₁-C₆ alkyl, —OH, —CO(C₁-C₆ alkyl), or aryl optionally substituted with one or more R¹⁴ independently selected from halogen, —NO₂, and —CN, or
R⁵ and R⁶, together with the atoms to which they are attached, form a 6-membered aryl optionally substituted with one or more R¹⁴ independently selected from halogen, —NO₂, and —CN.

17. The compound of any of claims 1-15, wherein Q is CR⁵, and R⁵ and R⁶ are each independently hydrogen, halogen, —NO₂, C₁-C₆ alkyl, —OH, or —CO(C₁-C₆ alkyl).

18. The compound of any of claims 1-15, wherein Q is CR⁵, and R⁵ and R⁶ are each independently hydrogen, halogen, C₁-C₆ alkyl, or —OH.

19. The compound of any of claims 1-15, wherein Q is CR⁵, and R⁵ is aryl, optionally substituted with one or more R¹⁴ independently selected from halogen, —NO₂, and —CN.

20. The compound of any of claims 1-19, wherein R³ and R⁶ are hydrogen.

21. The compound of any of claims 1-19, wherein
R³, R⁴, and R⁶ are hydrogen; and
Q is CR⁵, and R⁵ is halogen.

22. The compound of any of claims 1-19, wherein R⁶ is halogen or —OH.

23. The compound of any of claims 1-10, wherein
R³ and R⁶ are hydrogen;
R⁴ is hydrogen, halogen, or C₁-C₆ alkoxy;
Q is CR⁵, and R⁵ is hydrogen, halogen, —NO₂, C₁-C₆ alkyl, —CO(C₁-C₆ alkyl), or aryl optionally substituted with one or more R¹⁴ independently selected from halogen, —NO₂, and —CN; and
at least one of R⁴ and R⁵ is not hydrogen.

24. The compound of any of claims 1-23, wherein
Y is 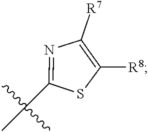
and
R⁷ and R⁸ are each independently hydrogen, —NO₂, or C₁-C₆ alkyl.

25. The compound of claim 24, wherein R⁷ and R⁸ are each independently hydrogen or C₁-C₆ alkyl.

26. The compound of claim 24, wherein R⁷ and R⁸ are each independently hydrogen or —NO₂.

27. The compound of any of claims 1-23, wherein
Y is 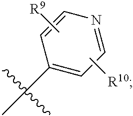
and
R⁹ and R¹⁰ are each independently hydrogen, —NO₂, or C₁-C₆ alkyl.

28. The compound of claim 27, wherein R⁹ and R¹⁰ are each independently hydrogen or halogen.

29. The compound of any of claims 1-23, wherein
Y is 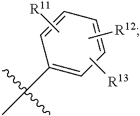
and
R¹¹, R¹², and R¹³ are each independently hydrogen, halogen, —NO₂, —CN, C₁-C₆ alkyl, C₁-C₆ haloalkyl, —NHCO(C₁-C₆ alkyl), C₁-C₆ alkoxy, —CON(C₁-C₆ alkyl)₂, or —S(O)₀₋₂NH₂, or
R¹¹ and R¹³, together with the atoms to which they are attached, form a 6-membered aryl optionally substituted with one or more R¹⁴ independently selected from halogen, —NO₂, and —CN.

30. The compound of any of claims 1-23, wherein
Y is 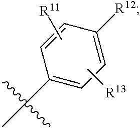
R¹¹ is hydrogen, halogen, or C₁-C₆ alkoxy;
R¹² is hydrogen, halogen, —NO₂, —CN, C₁-C₆ haloalkyl, or —S(O)₀₋₂NH₂;
R¹³ is hydrogen, halogen, —NO₂, or C₁-C₆ alkoxy; or
R¹¹ and R¹³, together with the atoms to which they are attached, form a 6-membered aryl; and
at least one of R¹¹, R¹², and R¹³ is not hydrogen.

31. The compound of any of claims 1-23, wherein
Y is 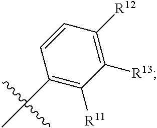
R¹¹ and R¹³ are each independently hydrogen, halogen, or C₁-C₆ alkoxy;
R¹² is hydrogen, halogen, —NO₂, —CN, C₁-C₆ haloalkyl, or —S(O)₀₋₂NH₂; and
at least one of R¹¹, R¹², and R¹³ is not hydrogen.

32. The compound of any of claims 29-31, wherein R¹¹ is halogen, R¹² is —NO₂, —CN, —F, or CF³, and R¹³ is hydrogen.

33. The compound of any of claims 29-31, wherein R¹¹ is hydrogen, R¹² is —NO₂, —CN, —F, or —CF³, and R¹³ is halogen.

34. The compound of any of claims 29-31, wherein R¹¹ is C₁-C₆ alkoxy, R¹² is —NO₂, and R¹³ is hydrogen.

35. The compound of any of claims 29-31, wherein R¹¹ is hydrogen, R¹² is —NO₂, and R¹³ is hydrogen.

36. The compound of any of claims 29-31, wherein R¹¹ is hydrogen, R¹² is —CF³, and R¹³ is hydrogen or halogen.

37. The compound of any of claims 29-31, wherein R¹¹ is halogen, R¹² is —CN, and R¹³ is hydrogen.

38. The compound of any of claims 1-23, wherein
Y is 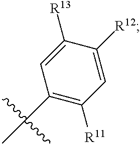
R¹¹ is hydrogen or halogen (e.g., —F, —Cl, or —Br);
R¹² is hydrogen, halogen, —NO₂, —CN, C₁-C₆ haloalkyl, or —S(O)₀₋₂NH₂;
R¹³ is hydrogen, halogen, or —NO₂; and
at least one of R¹¹, R¹², and R¹³ is not hydrogen.

39. The compound of claim 1, wherein
X is —C(O)NR^{1C}R^{2C}; and 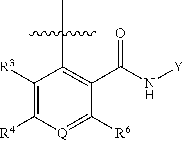
is
4-chloro-2-((2-chloro-4-nitrophenyl)carbamoyl)phenyl;
2-((2-chloro-4-nitrophenyl)carbamoyl)-4-fluorophenyl;
4-bromo-2-((2-chloro-4-nitrophenyl)carbamoyl)phenyl;
5-chloro-2-((2-chloro-4-nitrophenyl)carbamoyl)phenyl;
2-((2-chloro-4-nitrophenyl)carbamoyl)-5-methoxyphenyl;
2-((2-chloro-4-nitrophenyl)carbamoyl)-4-methoxyphenyl;
2-((2-chloro-4-nitrophenyl)carbamoyl)-4-methylphenyl;
2-((2-chloro-4-nitrophenyl)carbamoyl)-3-hydroxyphenyl;
2-((2-chloro-4-nitrophenyl)carbamoyl)-4-nitrophenyl;
4-acetyl-2-((2-chloro-4-nitrophenyl)carbamoyl)phenyl;
2-((2-chloro-4-nitrophenyl)carbamoyl)naphthalen-1-yl;
1-((2-chloro-4-nitrophenyl)carbamoyl)naphthalen-2-yl;
3-((2-chloro-4-nitrophenyl)carbamoyl)-2′,4′-difluoro-[1,1′-biphenyl]-4-yl;
4-chloro-2-((2-chlorophenyl)carbamoyl)phenyl;
4-chloro-2-((4-nitrophenyl)carbamoyl)phenyl;
4-chloro-2-((2,6-dichloro-4-nitrophenyl)carbamoyl)phenyl;
4-chloro-2-((3-chloro-4-nitrophenyl)carbamoyl)phenyl;
4-chloro-2-((2-methoxy-4-nitrophenyl)carbamoyl)phenyl;
4-chloro-2-((5-isopropylthiazol-2-yl)carbamoyl)phenyl;
4-chloro-2-((5-nitrothiazol-2-yl)carbamoyl)phenyl;
4-chloro-2-((3-chloropyridin-4-yl)carbamoyl)phenyl;
2-((4-amino-2-chlorophenyl)carbamoyl)-4-chlorophenyl;
4-chloro-2-((2-chloro-4-cyanophenyl)carbamoyl)phenyl;
4-chloro-2-((2-chloro-4-fluorophenyl)carbamoyl)phenyl;
4-chloro-2-((2,4-difluorophenyl)carbamoyl)phenyl;
4-chloro-2-((3-chloro-4-fluorophenyl)carbamoyl)phenyl;
4-chloro-2-((4-fluorophenyl)carbamoyl)phenyl;
4-chloro-2-((2,6-dichloro-4-fluorophenyl)carbamoyl)phenyl;
4-chloro-2-((4-sulfamoylphenyl)carbamoyl)phenyl;
4-chloro-2-((4-(trifluoromethyl)phenyl)carbamoyl)phenyl;
4-chloro-2-((2-chloro-4-(trifluoromethyl)phenyl)carbamoyl)phenyl;
4-chloro-2-((2-(trifluoromethyl)phenyl)carbamoyl)phenyl;
2-((4-acetamidophenyl)carbamoyl)-4-chlorophenyl;
4-chloro-2-((3-(dimethylcarbamoyl)phenyl)carbamoyl)phenyl;
4-chloro-2-((2,4-dichlorophenyl)carbamoyl)phenyl;
4-chloro-2-((4-chlorophenyl)carbamoyl)phenyl;
4-chloro-2-((4-chloro-2-fluoro-5-nitrophenyl)carbamoyl)phenyl;
4-chloro-2-((2,6-dichlorophenyl)carbamoyl)phenyl;
4-chloro-2-((2,6-difluorophenyl)carbamoyl)phenyl;
4-chloro-2-((4-methoxyphenyl)carbamoyl)phenyl;
4-chloro-2-((3-methoxyphenyl)carbamoyl)phenyl;
4-chloro-2-((2,3-dimethylphenyl)carbamoyl)phenyl;
4-chloro-2-((2-chloro-5-methoxyphenyl)carbamoyl)phenyl;
4-chloro-2-(p-tolylcarbamoyl)phenyl;
4-chloro-2-((2-nitrophenyl)carbamoyl)phenyl;
4-chloro-2-((6-nitronaphthalen-2-yl)carbamoyl)phenyl;
4-chloro-2-((4-nitronaphthalen-1-yl)carbamoyl)phenyl;
4-bromo-2-((4-(trifluoromethyl)phenyl)carbamoyl)phenyl;
4-bromo-2-((3-chloro-4-(trifluoromethyl)phenyl)carbamoyl)phenyl;
4-bromo-2-((3-fluoro-4-(trifluoromethyl)phenyl)carbamoyl)phenyl;
4-bromo-2-((2-chloro-4-(trifluoromethyl)phenyl)carbamoyl)phenyl;
4-bromo-2-((2-methoxy-4-nitrophenyl)carbamoyl)phenyl;
4-bromo-2-((3-chloro-4-nitrophenyl)carbamoyl)phenyl;
2-((2-chloro-4-(trifluoromethyl)phenyl)carbamoyl)naphthalen-1-yl;
2-((2-chloro-4-cyanophenyl)carbamoyl)naphthalen-1-yl;
3-((2-chloro-4-(trifluoromethyl)phenyl)carbamoyl)-2′,4′-difluoro-[1,1′-biphenyl]-4-yl;
3-((2-chloro-4-cyanophenyl)carbamoyl)-2′,4′-difluoro-[1,1′-biphenyl]-4-yl; or
3-((2-chloro-4-nitrophenyl)carbamoyl)quinolin-4-yl.

40. The compound of claim 39, wherein X is:

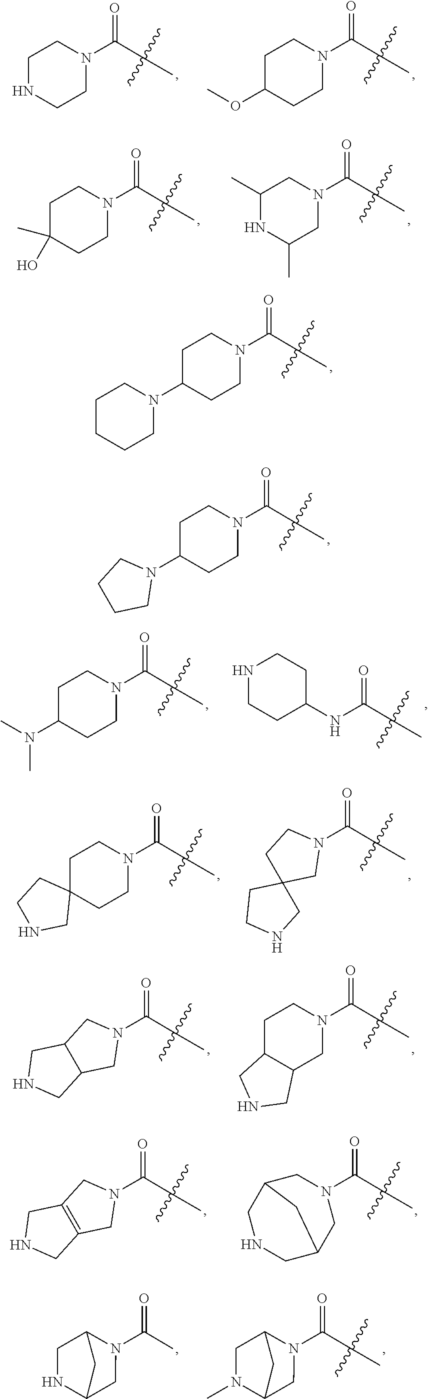

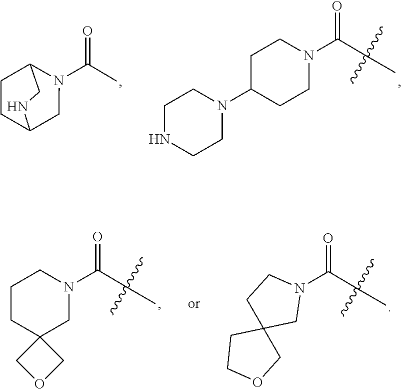

41. The compound of claim 1, wherein the compound is
4-bromo-2-((2-chloro-4-nitrophenyl)carbamoyl)phenyl piperazine-1-carboxylate;
4-chloro-2-((2-methoxy-4-nitrophenyl)carbamoyl)phenyl piperazine-1-carboxylate;
4-chloro-2-((4-nitrophenyl)carbamoyl)phenyl piperazine-1-carboxylate;
4-bromo-2-((3-fluoro-4-(trifluoromethyl)phenyl)carbamoyl)phenyl piperazine-1-carboxylate; or
a pharmaceutically acceptable salt thereof.

42. The compound of claim 1, wherein the compound is
4-chloro-2-((2-chloro-4-nitrophenyl)carbamoyl)phenyl 4-(trifluoromethyl)benzoate;
4-chloro-2-((2-chloro-4-nitrophenyl)carbamoyl)phenyl piperazine-1-carboxylate;
4-chloro-2-((2-chloro-4-nitrophenyl)carbamoyl)phenyl 2-methoxyacetate;
4-chloro-2-((2-chloro-4-nitrophenyl)carbamoyl)phenyl isopropyl carbonate;
4-chloro-2-((2-chloro-4-nitrophenyl)carbamoyl)phenyl isobutyl carbonate; or
a pharmaceutically acceptable salt thereof.

43. A pharmaceutical composition comprising one or more compounds according to any of claims 1-42 and a pharmaceutically acceptable carrier, excipient, adjuvant, and/or diluent.

44. A method of treating disease caused by severe acute respiratory syndrome coronavirus 2, comprising administering to a subject in need thereof a therapeutically effective amount of one or more compounds according to any of claims 1-42 or a pharmaceutical composition according to claim 43.

45. A method of treating disease caused by severe acute respiratory syndrome coronavirus 2, comprising administering to a subject in need thereof a therapeutically effective amount of
4-chloro-2-((2-chloro-4-nitrophenyl)carbamoyl)phenyl 4-methylpiperazine-1-carboxylate;
4-chloro-2-((2-chloro-4-nitrophenyl)carbamoyl)phenyl octanoate;
4-chloro-2-((2-chloro-4-nitrophenyl)carbamoyl)phenyl acetate; 4-chloro-2-((2-chloro-4-nitrophenyl)carbamoyl)phenyl benzoate;
4-chloro-2-((2-chloro-4-nitrophenyl)carbamoyl)phenyl diethyl phosphate; or
a pharmaceutically acceptable salt thereof; or
a pharmaceutical composition comprising
4-chloro-2-((2-chloro-4-nitrophenyl)carbamoyl)phenyl 4-methylpiperazine-1-carboxylate;
4-chloro-2-((2-chloro-4-nitrophenyl)carbamoyl)phenyl octanoate;
4-chloro-2-((2-chloro-4-nitrophenyl)carbamoyl)phenyl acetate; 4-chloro-2-((2-chloro-4-nitrophenyl)carbamoyl)phenyl benzoate;
4-chloro-2-((2-chloro-4-nitrophenyl)carbamoyl)phenyl diethyl phosphate; or
a pharmaceutically acceptable salt thereof; and
a pharmaceutically acceptable carrier, excipient, adjuvant, and/or diluent.

46. A method of treating disease caused by severe acute respiratory syndrome coronavirus 2, comprising administering to a subject in need thereof a therapeutically effective amount of one or more compounds of Formula II 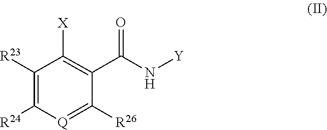
or a pharmaceutically acceptable salt thereof, wherein
X is —OR²¹, wherein
R²¹ is hydrogen, C₁-C₆ alkyl, C₁-C₆ haloalkyl, hydroxy(C₁-C₆ alkyl), alkoxy(C₁-C₆ alkyl), amino(C₁-C₆ alkyl), cycloalkyl, heterocycloalkyl,
or X is —NHR²², wherein
R²² is hydrogen, C₁-C₆ alkyl, C₁-C₆ haloalkyl, hydroxy(C₁-C₆ alkyl), alkoxy(C₁-C₆ alkyl), amino(C₁-C₆ alkyl), —CHO, —CO(C₁-C₆ alkyl), —S(O)₀₋₂(C₁-C₆ alkyl), or —S(O)₀₋₂(aryl);
R²³ and R²⁴ are each independently hydrogen, halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, C₁-C₆ haloalkoxy, —SH, —CONH₂, —CONH(C₁-C₆ alkyl), —CON(C₁-C₆ alkyl)₂, —CO₂H, —CO₂(C₁-C₆ alkyl), —CHO, —CO(C₁-C₆ alkyl), or —S(O)₀₋₂(C₁-C₆ alkyl), or
R²³ and R²⁴, together with the atoms to which they are attached, form a 5- or 6-membered aryl, heteroaryl, or heterocycyl optionally substituted with one or more R³⁴; or
X is —NHR²², and R²² and R²³, together with the atoms to which they are attached, form a 5- or 6-membered heteroaryl;
Q is N;
or Q is CR²⁵; and
R²⁵ is hydrogen, halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, C₁-C₆ haloalkoxy, —SH, —CONH₂, —CONH(C₁-C₆ alkyl), —CON(C₁-C₆ alkyl)₂, —CO₂H, —CO₂(C₁-C₆ alkyl), —CHO, —CO(C₁-C₆ alkyl), —S(O)₀₋₂(C₁-C₆ alkyl), cycloalkyl optionally substituted with one or more R³⁴, aryl optionally substituted with one or more R³⁴, heteroaryl optionally substituted with one or more R³⁴, or heterocycloalkyl optionally substituted with one or more R³⁴;
R²⁶ is hydrogen, halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, C₁-C₆ haloalkoxy, —SH, —CONH₂, —CONH(C₁-C₆ alkyl), —CON(C₁-C₆ alkyl)₂, —CO₂H, —CO₂(C₁-C₆ alkyl), —CHO, —CO(C₁-C₆ alkyl), —S(O)₀₋₂(C₁-C₆ alkyl), cycloalkyl optionally substituted with one or more R³⁴, aryl optionally substituted with one or more R³⁴, heteroaryl optionally substituted with one or more R³⁴, or heterocycloalkyl optionally substituted with one or more R³⁴, or
R²⁵ and R²⁶, together with the atoms to which they are attached, form a 5- or 6-membered aryl, heteroaryl, or heterocyclyl optionally substituted with one or more R³⁴;
Y is 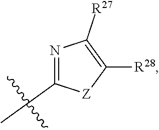
wherein
Z is O or S;
R²⁷ and R²¹ are each independently hydrogen, halogen, —NO₂, —CN, C₁-C₆ alkyl, or C₁-C₆ haloalkyl;
or Y is 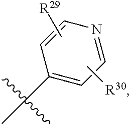
wherein
R²⁹ and R³⁰ are each independently hydrogen, halogen, —NO₂, —CN, C₁-C₆ alkyl, or C₁-C₆ haloalkyl;
or Y is 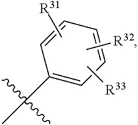
wherein
R³¹, R³², and R³³ are each independently hydrogen, halogen, —NO₂, —CN, C₁-C₆ alkyl, C₁-C₆ haloalkyl, —NH₂, —NH(C₁-C₆ alkyl), —N(C₁-C₆ alkyl)₂, —NHCO(C₁-C₆ alkyl), —N(C₁-C₆ alkyl)CO(C₁-C₆ alkyl), —OH, C₁-C₆ alkoxy, C₁-C₆ haloalkoxy, —SH, —CONH₂, —CONH(C₁-C₆ alkyl), —CON(C₁-C₆ alkyl)₂, —CO₂H, —CO₂(C₁-C₆ alkyl), —CHO, —CO(C₁-C₆ alkyl), —S(O)₀₋₂(C₁-C₆ alkyl), —S(O)₀₋₂NH₂, —S(O)₀₋₂NH(C₁-C₆ alkyl), or —S(O)₀₋₂N(C₁-C₆ alkyl)₂, or
R³¹ and R³³, together with the atoms to which they are attached, form a 5- or 6-membered aryl, heteroaryl, or heterocyclyl optionally substituted with one or more R³⁴; and
each R³⁴ is independently halogen, —NO₂, —CN, —NH₂, —NH(C₁-C₄ alkyl), —N(C₁-C₄ alkyl)₂, —OH, C₁-C₄ alkoxy, or C₁-C₄ haloalkoxy;
wherein the compound of Formula II is not 5-chloro-N-(2-chloro-4-nitrophenyl)-2-hydroxybenzamide.

47. The method of claim 46, wherein
X is —OR²¹; and
R²¹ is hydrogen, C₁-C₆ alkyl, amino(C₁-C₆ alkyl), or heterocycloalkyl.

48. The method of claim 46, wherein X is —OH.

49. The method of claim 46, wherein
X is —NHR²²; and
R²² is hydrogen, —CO(C₁-C₆ alkyl), —S(O)₀₋₂(C₁-C₆ alkyl), or —S(O)₀₋₂(aryl).

50. The method of claim 46, wherein
X is —NHR²²; and
R²² and R²³, together with the atoms to which they are attached, form a 5-membered heteroaryl.

51. The method of claim 46, wherein the compound is of Formula II-A

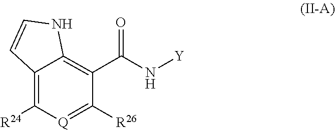

52. The method of any of claims 46-51, wherein Q is CR²⁵.

53. The method of any of claims 46-52, wherein
R²³ and R²⁴ are each independently hydrogen, halogen, or C₁-C₆ alkoxy, or
R²³ and R²⁴, together with the atoms to which they are attached, form a 6-membered aryl optionally substituted with one or more R³⁴ independently selected from halogen, —NO₂, and —CN.

54. The method of any of claims 46-52, wherein R²³ and R²⁴ are each independently hydrogen, halogen, or C₁-C₆ alkoxy.

55. The method of any of claims 46-52, wherein R²³ and R²⁴ are each independently hydrogen or halogen.

56. The method of any of claims 46-52, wherein R²³ is hydrogen and R²⁴ is halogen or C₁-C₆ alkoxy.

57. The method of any of claims 46-52, wherein R²³ and R²⁴ are each hydrogen.

58. The method of any of claims 46-57, wherein
Q is CR²⁵, and R²⁵ and R²⁶ are each independently hydrogen, halogen, —NO₂, C₁-C₆ alkyl, —OH, —CO(C₁-C₆ alkyl), or aryl optionally substituted with one or more R³⁴ independently selected from halogen, —NO₂, and —CN, or
R²⁵ and R²⁶, together with the atoms to which they are attached, form a 6-membered aryl optionally substituted with one or more R³⁴ independently selected from halogen, —NO₂, and —CN.

59. The method of any of claims 46-57, wherein Q is CR²⁵, and R²⁵ and R²⁶ are each independently hydrogen, halogen, —NO₂, C₁-C₆ alkyl, —OH, or —CO(C₁-C₆ alkyl).

60. The method of any of claims 46-57, wherein Q is CR²⁵, and R²⁵ and R²⁶ are each independently hydrogen, halogen, C₁-C₆ alkyl, or —OH.

61. The method of any of claims 46-57, wherein Q is CR²⁵, and R²⁵ is aryl, optionally substituted with one or more R³⁴ independently selected from halogen, —NO₂, and —CN.

62. The method of any of claims 46-61, wherein R²³ and R²⁶ are hydrogen.

63. The method of any of claims 46-61, wherein
R²³, R²⁴, and R²⁶ are hydrogen; and
Q is CR²⁵, and R²⁵ is halogen.

64. The method of any of claims 46-61, wherein R²⁶ is halogen or —OH.

65. The method of any of claims 46-52, wherein
R²³ and R²⁶ are hydrogen;
R²⁴ is hydrogen, halogen, or C₁-C₆ alkoxy; and
Q is CR²⁵, and R²⁵ is hydrogen, halogen, —NO₂, C₁-C₆ alkyl, —OH, —CO(C₁-C₆ alkyl), or aryl optionally substituted with one or more R³⁴ independently selected from halogen, —NO₂, and —CN;
wherein at least one of R²⁴ and R²⁵ is not hydrogen.

66. The method of any of claims 46-65, wherein
Y is 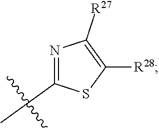
and
R²⁷ and R²⁸ are each independently hydrogen, —NO₂, or C₁-C₆ alkyl.

67. The method of claim 66, wherein R²⁷ and R²⁸ are each independently hydrogen or C₁-C₆ alkyl.

68. The method of claim 66, wherein R²⁷ and R²⁸ are each independently hydrogen or —NO₂.

69. The method of any of claims 46-65, wherein
Y is 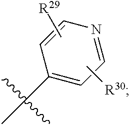
and
R²⁹ and R³⁰ are each independently hydrogen, —NO₂, or C₁-C₆ alkyl.

70. The method of claim 69, wherein R²⁹ and R³⁰ are each independently hydrogen or halogen.

71. The method of any of claims 46-65, wherein
Y is 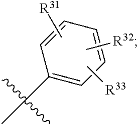
R³¹, R³², and R³³ are each independently hydrogen, halogen, —NO₂, —CN, C₁-C₆ alkyl, C₁-C₆ haloalkyl, —NHCO(C₁-C₆ alkyl), C₁-C₆ alkoxy, —CON(C₁-C₆ alkyl)₂, or —S(O)₀₋₂NH₂, or
R³¹ and R³³, together with the atoms to which they are attached, form a 6-membered aryl optionally substituted with one or more R³⁴ independently selected from halogen, —NO₂, and —CN.

72. The method of any of claims 46-65, wherein
Y is 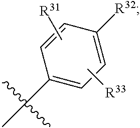
R³¹ is hydrogen, halogen, or C₁-C₆ alkoxy;
R³² is hydrogen, halogen, —NO₂, —CN, C₁-C₆ haloalkyl, or —S(O)₀₋₂NH₂
R³³ is hydrogen, halogen, —NO₂, or C₁-C₆ alkoxy; or
R³¹ and R³³, together with the atoms to which they are attached, form a 6-membered aryl; and
at least one of R³¹, R³², and R³³ is not hydrogen.

73. The method of any of claims 46-65, wherein
Y is 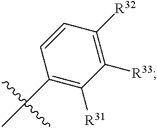
R³¹ and R³³ are each independently hydrogen, halogen, or C₁-C₆ alkoxy;
R³² is hydrogen, halogen, —NO₂, —CN, C₁-C₆ haloalkyl, or —S(O)₀₋₂NH₂; and
at least one of R³¹, R³², and R³³ is not hydrogen.

74. The method of any of claims 71-73, wherein R³¹ is halogen, R³² is —NO₂, and R³³ is hydrogen.

75. The method of any of claims 71-73, wherein R³¹ is hydrogen, R³² is —NO₂, —CN, —F, or —CF³, and R³³ is halogen.

76. The method of any of claims 71-73, wherein R³¹ is C₁-C₆ alkoxy, R³² is —NO₂, and R³³ is hydrogen.

77. The method of any of claims 71-73, wherein R³¹ is hydrogen, R³² is —NO₂, and R³³ is hydrogen.

78. The method of any of claims 71-73, wherein R³¹ is hydrogen, R³² is —CF³, and R³³ is hydrogen or halogen.

79. The method of any of claims 71-73, wherein R³¹ is halogen, R³² is —CN, and R³³ is hydrogen.

80. The method of any of claims 46-65, wherein
Y is 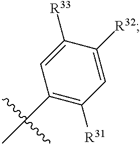
R³¹ is hydrogen or halogen (e.g., —F, —Cl, or —Br);
R³² is hydrogen, halogen, —NO₂, —CN, C₁-C₆ haloalkyl, or —S(O)₀₋₂NH₂;
R³³ is hydrogen, halogen, or —NO₂; and
at least one of R³¹, R³², and R³³ is not hydrogen.

81. The method of claim 46, wherein the compound is
5-chloro-N-(2-chloro-4-nitrophenyl)-2-hydroxybenzamide;
5-chloro-N-(2-chloro-4-nitrophenyl)-2-methoxybenzamide;
2-(2-aminoethoxy)-5-chloro-N-(2-chloro-4-nitrophenyl)benzamide;
5-chloro-N-(2-chloro-4-nitrophenyl)-2-(piperidin-4-yloxy)benzamide;
N-(2-chloro-4-nitrophenyl)-5-fluoro-2-hydroxybenzamide;
5-bromo-N-(2-chloro-4-nitrophenyl)-2-hydroxybenzamide;
4-chloro-N-(2-chloro-4-nitrophenyl)-2-hydroxybenzamide;
N-(2-chloro-4-nitrophenyl)-2-hydroxy-4-methoxybenzamide;
N-(2-chloro-4-nitrophenyl)-2-hydroxy-5-methoxybenzamide;
N-(2-chloro-4-nitrophenyl)-2-hydroxy-5-methylbenzamide;
N-(2-chloro-4-nitrophenyl)-2,6-dihydroxybenzamide;
N-(2-chloro-4-nitrophenyl)-2-hydroxy-5-nitrobenzamide;
5-acetyl-N-(2-chloro-4-nitrophenyl)-2-hydroxybenzamide;
N-(2-chloro-4-nitrophenyl)-1-hydroxy-2-naphthamide;
N-(2-chloro-4-nitrophenyl)-2-hydroxy-1-naphthamide;
N-(2-chloro-4-nitrophenyl)-2′,4′-difluoro-4-hydroxy-[1,1′-biphenyl]-3-carboxamide;
2-amino-5-chloro-N-(2-chloro-4-nitrophenyl)benzamide;
2-amino-6-chloro-N-(2-chloro-4-nitrophenyl)benzamide;
2-acetamido-N-(2-chloro-4-nitrophenyl)benzamide;
5-bromo-N-(2-chloro-4-nitrophenyl)-2-(methylsulfonamido)benzamide;
5-chloro-N-(2-chloro-4-nitrophenyl)-2-(phenylsulfonamido)benzamide;
N-(2-chloro-4-nitrophenyl)-1H-indole-7-carboxamide;
5-chloro-N-(2-chlorophenyl)-2-hydroxybenzamide;
5-chloro-2-hydroxy-N-(4-nitrophenyl)benzamide;
5-chloro-N-(2,6-dichloro-4-nitrophenyl)-2-hydroxybenzamide;
5-chloro-N-(3-chloro-4-nitrophenyl)-2-hydroxybenzamide;
5-chloro-2-hydroxy-N-(2-methoxy-4-nitrophenyl)benzamide;
5-chloro-2-hydroxy-N-(5-isopropylthiazol-2-yl)benzamide;
5-chloro-2-hydroxy-N-(5-nitrothiazol-2-yl)benzamide;
5-chloro-N-(3-chloropyridin-4-yl)-2-hydroxybenzamide;
N-(4-amino-2-chlorophenyl)-5-chloro-2-hydroxybenzamide;
5-chloro-N-(2-chloro-4-cyanophenyl)-2-hydroxybenzamide;
5-chloro-N-(2-chloro-4-fluorophenyl)-2-hydroxybenzamide;
5-chloro-N-(2,4-difluorophenyl)-2-hydroxybenzamide;
5-chloro-N-(3-chloro-4-fluorophenyl)-2-hydroxybenzamide;
5-chloro-N-(4-fluorophenyl)-2-hydroxybenzamide;
5-chloro-N-(2,6-dichloro-4-fluorophenyl)-2-hydroxybenzamide;
5-chloro-2-hydroxy-N-(4-sulfamoylphenyl)benzamide;
5-chloro-2-hydroxy-N-(4-(trifluoromethyl)phenyl)benzamide;
5-chloro-N-(2-chloro-4-(trifluoromethyl)phenyl)-2-hydroxybenzamide;
5-chloro-2-hydroxy-N-(2-(trifluoromethyl)phenyl)benzamide;
N-(4-acetamidophenyl)-5-chloro-2-hydroxybenzamide;
5-chloro-N-(3-(dimethylcarbamoyl)phenyl)-2-hydroxybenzamide;
5-chloro-N-(2,4-dichlorophenyl)-2-hydroxybenzamide;
5-chloro-N-(4-chlorophenyl)-2-hydroxybenzamide;
5-chloro-N-(4-chloro-2-fluoro-5-nitrophenyl)-2-hydroxybenzamide;
5-chloro-N-(2,6-dichlorophenyl)-2-hydroxybenzamide;
5-chloro-N-(2,6-difluorophenyl)-2-hydroxybenzamide;
5-chloro-2-hydroxy-N-(4-methoxyphenyl)benzamide;
5-chloro-2-hydroxy-N-(3-methoxyphenyl)benzamide;
5-chloro-N-(2,3-dimethylphenyl)-2-hydroxybenzamide;
5-chloro-N-(2-chloro-5-methoxyphenyl)-2-hydroxybenzamide;
5-chloro-2-hydroxy-N-(p-tolyl)benzamide;
5-chloro-2-hydroxy-N-(2-nitrophenyl)benzamide;
5-chloro-2-hydroxy-N-(6-nitronaphthalen-2-yl)benzamide;
5-chloro-2-hydroxy-N-(4-nitronaphthalen-1-yl)benzamide;
5-bromo-2-hydroxy-N-(4-(trifluoromethyl)phenyl)benzamide;
5-bromo-N-(3-chloro-4-(trifluoromethyl)phenyl)-2-hydroxybenzamide;
5-bromo-N-(3-fluoro-4-(trifluoromethyl)phenyl)-2-hydroxybenzamide;
5-bromo-N-(2-chloro-4-(trifluoromethyl)phenyl)-2-hydroxybenzamide;
5-bromo-2-hydroxy-N-(2-methoxy-4-nitrophenyl)benzamide;
5-bromo-N-(3-chloro-4-nitrophenyl)-2-hydroxybenzamide;
N-(2-chloro-4-(trifluoromethyl)phenyl)-1-hydroxy-2-naphthamide;
N-(2-chloro-4-cyanophenyl)-1-hydroxy-2-naphthamide;
N-(2-chloro-4-(trifluoromethyl)phenyl)-2′,4′-difluoro-4-hydroxy-[1,1′-biphenyl]-3-carboxamide;
N-(2-chloro-4-cyanophenyl)-2′,4′-difluoro-4-hydroxy-[1,1′-biphenyl]-3-carboxamide;
N-(2-chloro-4-cyanophenyl)-1H-indole-7-carboxamide;
N-(2-chloro-4-nitrophenyl)-4-hydroxyquinoline-3-carboxamide; or
a pharmaceutically acceptable salt thereof.

82. The method of claim 46, wherein the compound is
5-bromo-N-(2-chloro-4-nitrophenyl)-2-hydroxybenzamide;
5-chloro-N-(2-chloro-4-cyanophenyl)-2-hydroxybenzamide;
5-chloro-2-hydroxy-N-(4-(trifluoromethyl)phenyl)benzamide;
N-(2-chloro-4-nitrophenyl)-1H-indole-7-carboxamide;
5-chloro-2-hydroxy-N-(2-methoxy-4-nitrophenyl)benzamide;
5-chloro-2-hydroxy-N-(4-nitrophenyl)benzamide;
5-bromo-N-(3-fluoro-4-(trifluoromethyl)phenyl)-2-hydroxybenzamide; or
a pharmaceutically acceptable salt thereof.

83. The method of any of claims 46-82, wherein the one or more compounds is provided as a pharmaceutical composition comprising the one or more compounds and a pharmaceutically acceptable carrier, excipient, adjuvant, and/or diluent.

84. The method of any of claims 44-83, further comprising administering one or more secondary therapeutic agents.

85. The method of claim 84, wherein the secondary therapeutic agent is selected from a steroid, nonsteroidal anti-inflammatory drug, immunomodulating agent, chemotherapy agent, intravenous gamma globulin, vascular health agent, convalescent plasma, viral-targeting antiviral, interferon, host-targeting compound, and other agent with an antiviral activity (e.g., having an unknown mechanism of action, such as, but not limited to, ciclesonide, nitazoxanide, and emetine).

86. The method of any of claims 44-85, wherein the one or more compounds or the pharmaceutical composition are administered orally.

87. The method of any of claims 44-85, wherein the one or more compounds or the pharmaceutical composition are administered intravenously.

88. The method of any of claims 44-87, wherein the subject is a mammal.

89. The method of claim 88, wherein the subject is a human.
